SEQUENCE LISTING (58 sequences)

SEQ ID NO: 1 - protein, 5 aa, synthetic construct
SEAMN

SEQ ID NO: 2 - protein, 16 aa, synthetic construct
YIYTSGTTYRANWARG

SEQ ID NO: 3 - protein, 14 aa, synthetic construct
GTGIYDYYYWVMDL

SEQ ID NO: 4 - protein, 11 aa, synthetic construct
QASQSISTYLA

SEQ ID NO: 5 - protein, 7 aa, synthetic construct
AASTLES

SEQ ID NO: 6 - protein, 10 aa, synthetic construct
QSYSDGDSVG

SEQ ID NO: 7 - protein, 122 aa, synthetic construct
QVQLVESGGGVVQPGRSLRLSCAASGFTFSSEAMNWIRQPPGKGLEWIGYIYTSGTTYRANWARGRFTISRDNSKNTLYLQMNSLRAEDTAVYYCARGTGIYDYYYWVMDLWGPGTLVTVSS

SEQ ID NO: 8 - protein, 108 aa, synthetic construct
DIQMTQSPSSLSASVGDRVTITCQASQSISTYLAWYQQKPGQPPKLLIYAASTLESGVPSRFSGSGSGTDFTLTISSLQPEDFATYYCQSYSDGDSVGFGQGTKVEIK

SEQ ID NO: 9 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 10 - protein, 107 aa, synthetic construct [sequence omitted]

SEQ ID NO: 11 - protein, 450 aa, synthetic construct
QVQLVESGGGVVQPGRSLRLSCAASGFTFSSEAMNWIRQPPGKGLEWIGYIYTSGTTYRANWARGRFTISRDNSKNTLYLQMNSLRAEDTAVYYCARGTGIYDYYYWVMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 12 - protein, 215 aa, synthetic construct
DIQMTQSPSSLSASVGDRVTITCQASQSISTYLAWYQQKPGQPPKLLIYAASTLESGVPSRFSGSGSGTDFTLTISSLQPEDFATYYCQSYSDGDSVGFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 5 aa, synthetic construct
SYAMN

SEQ ID NO: 14 - protein, 16 aa, synthetic construct
YIYTSGTTYHANWARG

SEQ ID NO: 15 - protein, 14 aa, synthetic construct
GTFIWDYYYWVMDL

SEQ ID NO: 16 - protein, 11 aa, synthetic construct
QASQSISTYLA

SEQ ID NO: 17 - protein, 7 aa, synthetic construct
KASTLES

SEQ ID NO: 18 - protein, 10 aa, synthetic construct
QSYSDADSVG

SEQ ID NO: 19 - protein, 5 aa, synthetic construct
SYAMN

SEQ ID NO: 20 - protein, 16 aa, synthetic construct
YIYTSGTRYRANWARG

SEQ ID NO: 21 - protein, 14 aa, synthetic construct
GTDIYDYYYWVMDL

SEQ ID NO: 22 - protein, 11 aa, synthetic construct
QASQSISTYLA

SEQ ID NO: 23 - protein, 7 aa, synthetic construct
AASTLES

SEQ ID NO: 24 - protein, 10 aa, synthetic construct
QSYSDGDSVG

SEQ ID NO: 25 - protein, 5 aa, synthetic construct
SYAMN

SEQ ID NO: 26 - protein, 16 aa, synthetic construct
YIYTSGTRYRANWARG

SEQ ID NO: 27 - protein, 14 aa, synthetic construct
GTDIYDYYYWVMDL

SEQ ID NO: 28 - protein, 11 aa, synthetic construct
QASQSISTYLA

SEQ ID NO: 29 - protein, 7 aa, synthetic construct
AASTLES

SEQ ID NO: 30 - protein, 10 aa, synthetic construct
QSYSDADSVG

SEQ ID NO: 31 - protein, 122 aa, synthetic construct
QVQLVESGGGVVQPGRSLRLSCAASGFTFSSYAMNWIRQPPGKGLEWIGYIYTSGTTYHANWARGRFTISRDNSKNTLYLQMNSLRAEDTAVYYCARGTFIWDYYYWVMDLWGPGTLVTVSS

SEQ ID NO: 32 - protein, 108 aa, synthetic construct
DIQMTQSPSSLSASVGDRVTITCQASQSISTYLAWYQQKPGQPPKLLIYKASTLESGVPSRFSGSGSGTDFTLTISSLQPEDFATYYCQSYSDADSVGFGQGTKVEIK

SEQ ID NO: 33 - protein, 122 aa, synthetic construct
QVQLVESGGGVVQPGRSLRLSCAASGFTFSSYAMNWIRQPPGKGLEWIGYIYTSGTRYRANWARGRFTISRDNSKNTLYLQMNSLRAEDTAVYYCARGTDIYDYYYWVMDLWGPGTLVTVSS

SEQ ID NO: 34 - protein, 108 aa, synthetic construct; identical to SEQ ID NO: 8

SEQ ID NO: 35 - protein, 122 aa, synthetic construct; identical to SEQ ID NO: 33

SEQ ID NO: 36 - protein, 108 aa, synthetic construct
DIQMTQSPSSLSASVGDRVTITCQASQSISTYLAWYQQKPGQPPKLLIYAASTLESGVPSRFSGSGSGTDFTLTISSLQPEDFATYYCQSYSDADSVGFGQGTKVEIK

SEQ ID NO: 37 - protein, 450 aa, synthetic construct
QVQLVESGGGVVQPGRSLRLSCAASGFTFSSYAMNWIRQPPGKGLEWIGYIYTSGTTYHANWARGRFTISRDNSKNTLYLQMNSLRAEDTAVYYCARGTFIWDYYYWVMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 38 - protein, 215 aa, synthetic construct
DIQMTQSPSSLSASVGDRVTITCQASQSISTYLAWYQQKPGQPPKLLIYKASTLESGVPSRFSGSGSGTDFTLTISSLQPEDFATYYCQSYSDADSVGFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 450 aa, synthetic construct
QVQLVESGGGVVQPGRSLRLSCAASGFTFSSEAMNWIRQPPGKGLEWIGYIYTSGTTYRANWARGRFTISRDNSKNTLYLQMNSLRAEDTAVYYCARGTGIYDYYYWVMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 40 - protein, 215 aa, synthetic construct; identical to SEQ ID NO: 12

SEQ ID NO: 41 - protein, 450 aa, synthetic construct
QVQLVESGGGVVQPGRSLRLSCAASGFTFSSYAMNWIRQPPGKGLEWIGYIYTSGTRYRANWARGRFTISRDNSKNTLYLQMNSLRAEDTAVYYCARGTDIYDYYYWVMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 42 - protein, 215 aa, synthetic construct; identical to SEQ ID NO: 12

SEQ ID NO: 43 - protein, 450 aa, synthetic construct; identical to SEQ ID NO: 41

SEQ ID NO: 44 - protein, 215 aa, synthetic construct
DIQMTQSPSSLSASVGDRVTITCQASQSISTYLAWYQQKPGQPPKLLIYAASTLESGVPSRFSGSGSGTDFTLTISSLQPEDFATYYCQSYSDADSVGFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 450 aa, synthetic construct
QVQLVESGGGVVQPGRSLRLSCAASGFTFSSYAMNWIRQPPGKGLEWIGYIYTSGTTYHANWARGRFTISRDNSKNTLYLQMNSLRAEDTAVYYCARGTFIWDYYYWVMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 215 aa, synthetic construct
DIQMTQSPSSLSASVGDRVTITCQASQSISTYLAWYQQKPGQPPKLLIYKASTLESGVPSRFSGSGSGTDFTLTISSLQPEDFATYYCQSYSDADSVGFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 47 - protein, 450 aa, synthetic construct; identical to SEQ ID NO: 11

SEQ ID NO: 48 - protein, 215 aa, synthetic construct
DIQMTQSPSSLSASVGDRVTITCQASQSISTYLAWYQQKPGQPPKLLIYAASTLESGVPSRFSGSGSGTDFTLTISSLQPEDFATYYCQSYSDGDSVGFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 49 - protein, 450 aa, synthetic construct
QVQLVESGGGVVQPGRSLRLSCAASGFTFSSYAMNWIRQPPGKGLEWIGYIYTSGTRYRANWARGRFTISRDNSKNTLYLQMNSLRAEDTAVYYCARGTDIYDYYYWVMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 50 - protein, 215 aa, synthetic construct; identical to SEQ ID NO: 48

SEQ ID NO: 51 - protein, 450 aa, synthetic construct; identical to SEQ ID NO: 49

SEQ ID NO: 52 - protein, 215 aa, synthetic construct
DIQMTQSPSSLSASVGDRVTITCQASQSISTYLAWYQQKPGQPPKLLIYAASTLESGVPSRFSGSGSGTDFTLTISSLQPEDFATYYCQSYSDADSVGFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 54 - RNA, 1173 nt, Homo sapiens
  source 1..1173: mol_type = mRNA
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 55 - protein, 390 aa, Mus musculus
MPPSGLRLLPLLLPLPWLLVLTPGRPAAGLSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 56 - RNA, 1173 nt, Mus musculus
  source 1..1173: mol_type = mRNA
atgccgccctcggggctgcggctactgccgcttctgctcccactcccgtggcttctagtgctgacgcccgggaggccagccgcgggactctccacctgcaagaccatcgacatggagctggtgaaacggaagcgcatcgaagccatccgtggccagatcctgtccaaactaaggctcgccagtcccccaagccagggggaggtaccgcccggcccgctgcccgaggcggtgctcgctttgtacaacagcacccgcgaccgggtggcaggcgagagcgccgacccagagccggagcccgaagcggactactatgctaaagaggtcacccgcgtgctaatggtggaccgcaacaacgccatctatgagaaaaccaaagacatctcacacagtatatatatgttcttcaatacgtcagacattcgggaagcagtgcccgaacccccattgctgtcccgtgcagagctgcgcttgcagagattaaaatcaagtgtggagcaacatgtggaactctaccagaaatatagcaacaattcctggcgttaccttggtaaccggctgctgacccccactgatacgcctgagtggctgtcttttgacgtcactggagttgtacggcagtggctgaaccaaggagacggaatacagggctttcgattcagcgctcactgctcttgtgacagcaaagataacaaactccacgtggaaatcaacgggatcagccccaaacgtcggggcgacctgggcaccatccatgacatgaaccggcccttcctgctcctcatggccacccccctggaaagggcccagcacctgcacagctcacggcaccggagagccctggataccaactattgcttcagctccacagagaagaactgctgtgtgcggcagctgtacattgactttaggaaggacctgggttggaagtggatccacgagcccaagggctaccatgccaacttctgtctgggaccctgcccctatatttggagcctggacacacagtacagcaaggtccttgccctctacaaccaacacaacccgggcgcttcggcgtcaccgtgctgcgtgccgcaggctttggagccactgcccatcgtctactacgtgggtcgcaagcccaaggtggagcagttgtccaacatgattgtgcgctcctgcaagtgcagctga

SEQ ID NO: 57 - protein, 390 aa, Macaca fascicularis
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSKDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 58 - RNA, 1173 nt, Macaca fascicularis
  source 1..1173: mol_type = mRNA
atgccgccctccgggctgcggctgctgccgctgctgctaccgctgctgtggctactggtgctgacgcctggccggccggccgccggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcggagccggaacccgaaccggaggccgactactacgccaaggaggtcacccgcgtgctaatggtggaaacccacaacgaaatctatgacaagttcaagcagagcacacacagcatatatatgttcttcaacacatcagagctccgagaagcagtacctgaacctgtgttgctctcccgggcagagctgcgtctgctgaggctcaagttaaaagtggagcagcatgtggagctgtaccagaaatacagcaacaattcctggcgatacctcagcaaccggctgctggcgcccagcgactcgccggagtggttgtcttttgatgtcaccggagttgtgcggcagtggttgagccgcggaggggaaattgagggctttcgccttagcgcccactgctcctgtgacagcaaagataacacactgcaagtggacatcaacgggttcactaccggccgccgaggtgacctggccaccattcatggcatgaaccggcctttcctgcttctcatggccaccccgctggagagggcccaacatctgcaaagctcccggcaccgccgagccctggacaccaactactgcttcagctccacggagaagaactgctgcgtgcggcagctgtatattgacttccgcaaggacctcggctggaagtggatccacgagcccaagggctaccatgccaacttctgcctgggaccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaaatgcagctga

(19) United States
(12) Patent Application Publication
(10) Pub. No.: US 2026/0308241 A1
(43) Pub. Date: Oct. 8, 2026

(54) METHODS AND COMPOSITIONS FOR TREATING CANCER

(71) Applicants: Genentech, Inc., South San Francisco, CA (US); Chugai Seiyaku Kabushiki Kaisha, Tokyo (JP)

(72) Inventors: Colby Sasson SHEMESH, San Diego, CA (US); David VOONG, San Francisco, CA (US); Yulei WANG, Foster City, CA (US); Eunpi CHO, Palo Alto, CA (US); Stevan Nicholas DJAKOVIC, San Francisco, CA (US); Eisuke UEDA, Tokyo (JP); Takaaki ISHIDA, Tokyo (JP)

(21) Appl. No.: 19/483,179

(22) PCT Filed: May 8, 2024
(86) PCT No.: PCT/US2024/028339

§ 371 Date: Nov. 10, 2025

Related U.S. Application Data
(60) Provisional application No. 63/465,498, filed on May 10, 2023.

(51) Int. Cl. C07K 16/22; A61K 39/00; A61P 35/00; C07K 16/28

(52) U.S. Cl. CPC C07K 16/22; A61P 35/00; C07K 16/2827; A61K 2039/507; A61K 2039/545; C07K 2317/56; C07K 2317/76

(57) ABSTRACT

The invention provides methods and compositions for treating cancer (e.g., a locally advanced, recurrent, or metastatic solid tumor) in a subject, for example, by administering an anti-cancer therapy that includes an anti-latent TGF-beta 1 antibody to the subject. Also provided are compositions (e.g., an anti-latent TGF-beta 1 antibody, pharmaceutical compositions thereof, kits thereof, and articles of manufacture thereof) for use in treating cancer in a subject.

### SEQUENCE LISTING

The instant application contains a Sequence Listing which has been submitted electronically in XML format and is hereby incorporated by reference in its entirety. Said XML copy, created on Apr. 29, 2024, is named “50474-323WO2_Sequence_Listing_4_29_24” and is 62,842 bytes in size.

### FIELD OF THE INVENTION

This invention relates to methods and compositions for use in treating cancer (e.g., a locally advanced, recurrent, or metastatic solid tumor) in a subject, for example, by administering to the subject an anti-cancer therapy that includes an anti-latent transforming growth factor (TGF)-beta 1 antibody.

### BACKGROUND OF THE INVENTION

Cancer remains one of the deadliest threats to human health. In the U.S., cancer affects nearly 1.3 million new patients each year and is the second leading cause of death after heart disease, accounting for approximately 1 in 4 deaths. It is also predicted that cancer may surpass cardiovascular diseases as the number one cause of death within 5 years. Solid tumors are responsible for most of those deaths. Although there have been significant advances in the medical treatment of certain cancers, the overall 5-year survival rate for all cancers has improved only by about 10% in the past 20 years. Malignant solid tumors, in particular, metastasize and grow rapidly in an uncontrolled manner, making their timely detection and treatment extremely difficult.

Despite the significant advancement in the treatment of cancer, improved anti-cancer therapies are still being sought.

### SUMMARY OF THE INVENTION

The invention provides, inter alia, methods for treating cancer (e.g., a locally advanced, recurrent, or metastatic solid tumor), and related compositions for use and articles of manufacture.

In one aspect, the invention provides a method of treating a subject having a locally advanced, recurrent, or metastatic solid tumor, the method comprising administering to the subject an anti-cancer therapy comprising an anti-latent transforming growth factor (TGF)-beta 1 antibody at a dosage of 1800 mg, wherein the anti-latent TGF-beta 1 antibody comprises the following six hypervariable regions (HVRs): (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another aspect, the invention provides an anti-latent TGF-beta 1 for use in treatment of a subject having a locally advanced, recurrent, or metastatic solid tumor, the treatment comprising administering to the subject an anti-cancer therapy comprising an anti-latent TGF-beta 1 antibody at a dosage of 1800 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some aspects, the anti-latent TGF-beta 1 antibody is administered to the subject in a dosing regimen comprising one or more 21-day dosing cycles.

In some aspects, the anti-latent TGF-beta 1 antibody is administered to the subject on Day 1 of each 21-day dosing cycle.

In another aspect, the invention provides a method of treating a subject having a locally advanced, recurrent, or metastatic solid tumor, the method comprising administering to the subject an anti-cancer therapy comprising an anti-latent TGF-beta 1 antibody at a dosage of 1800 mg every three weeks (Q3W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another aspect, the invention provides an anti-latent TGF-beta 1 for use in treatment of a subject having a locally advanced, recurrent, or metastatic solid tumor, the treatment comprising administering to the subject an anti-cancer therapy comprising an anti-latent TGF-beta 1 antibody at a dosage of 1800 mg every three weeks (Q3W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another aspect, the invention provides a method of treating a subject having a locally advanced, recurrent, or metastatic solid tumor, the method comprising administering to the subject an anti-cancer therapy comprising an anti-latent TGF-beta 1 antibody at a dosage of 1200 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another aspect, the invention provides an anti-latent TGF-beta 1 for use in treatment of a subject having a locally advanced, recurrent, or metastatic solid tumor, the treatment comprising administering to the subject an anti-cancer therapy comprising an anti-latent TGF-beta 1 antibody at a dosage of 1200 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some aspects, the anti-latent TGF-beta 1 antibody is administered to the subject in a dosing regimen comprising one or more 28-day dosing cycles.

In some aspects, the anti-latent TGF-beta 1 antibody is administered to the subject on Days 1 and 15 of each 28-day dosing cycle.

In another aspect, the invention provides a method of treating a subject having a locally advanced, recurrent, or metastatic solid tumor, the method comprising administering to the subject an anti-cancer therapy comprising an anti-latent TGF-beta 1 antibody at a dosage of 1200 mg every two weeks (Q2W), wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another aspect, the invention provides an anti-latent TGF-beta 1 for use in treatment of a subject having a locally advanced, recurrent, or metastatic solid tumor, the treatment comprising administering to the subject an anti-cancer therapy comprising an anti-latent TGF-beta 1 antibody at a dosage of 1200 mg every two weeks (Q2W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some aspects, the anti-latent TGF-beta 1 antibody is administered to the subject intravenously.

In some aspects, the anti-latent TGF-beta 1 antibody is administered to the subject intravenously by infusion.

In some aspects, a tumor sample from the subject has been determined to have a detectable level of PD-L1 expression.

In some aspects, the subject's age is 18 years or older.

In some aspects, the anti-cancer therapy is a first-line therapy.

In some aspects, the anti-cancer therapy is a second-line therapy or a third-line therapy.

In some aspects, the subject has not been previously treated with a checkpoint inhibitor.

In some aspects, the solid tumor is metastatic.

In some aspects, the locally advanced, recurrent, or metastatic solid tumor is non-small cell lung cancer (NSCLC), gastric cancer, pancreatic ductal adenocarcinoma (PDAC), urothelial carcinoma (UC), gastrointestinal stromal tumor (GIST), skin cancer, colorectal cancer, ovarian (OV) cancer, renal cancer, or gallbladder cancer.

In some aspects, the locally advanced, recurrent, or metastatic solid tumor is NSCLC.

In some aspects, the NSCLC is histologically or cytologically confirmed metastatic nonsquamous NSCLC or metastatic squamous NSCLC.

In some aspects, the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

In some aspects, the subject has previously received a combination therapy comprising a platinum-containing chemotherapy regimen and a PD-1 axis binding antagonist.

In some aspects, the subject has previously received a platinum-containing chemotherapy regimen and a PD-1 axis binding antagonist as individual regimens.

In some aspects, the subject had disease progression or recurrence within 6 months of definitive therapy for locally advanced NSCLC.

In some aspects, a tumor sample from the subject has been determined to have a detectable level of PD-L1 expression.

In some aspects, the locally advanced, recurrent, or metastatic solid tumor is gastric cancer.

In some aspects, the subject has unresectable locally advanced or metastatic gastric cancer that is histologically confirmed to be adenocarcinoma.

In some aspects, the gastric cancer comprises esophagogastric junction cancer.

In some aspects, the gastric cancer is HER2-negative gastric cancer.

In some aspects, the subject is previously untreated for gastric cancer and/or the subject has not been previously treated with a checkpoint inhibitor.

In some aspects, the locally advanced, recurrent, or metastatic solid tumor is PDAC.

In some aspects, the subject has histologically or cytologically confirmed metastatic PDAC.

In some aspects, the subject is previously untreated for the PDAC and/or the subject has not been previously treated with a checkpoint inhibitor.

In some aspects, the locally advanced, recurrent, or metastatic solid tumor is UC.

In some aspects, the subject has histologically documented, locally advanced (T4b, any N; or any T, N2-N3) UC, or metastatic UC (M1, Stage 4).

In some aspects, the subject is previously untreated for UC.

In some aspects, the subject is ineligible for cisplatin-containing chemotherapy.

In some aspects, the subject is ineligible for cisplatin-containing chemotherapy as defined by any one of the following criteria: (i) impaired renal function in terms of glomerular filtration rate (GFR) of ≥30 mL/min but <60 mL/min as assessed by direct measurement or by calculation from serum or plasma creatinine; (ii) hearing loss of 25 dB at two contiguous frequencies as measured by audiometry; (iii) Grade 2 peripheral neuropathy; or (iv) Eastern Cooperative Oncology Group (ECOG) Performance Status of 2.

In some aspects, the subject has previously received at least one platinum-containing chemotherapy regimen.

In some aspects, the subject had disease progression during or following treatment with at least one platinum-containing chemotherapy regimen.

In some aspects, the at least one platinum-containing chemotherapy regimen comprised (i) gemcitabine and cisplatin or carboplatin or (ii) methotrexate, vinblastine, doxorubicin, and cisplatin.

In some aspects, the subject received prior adjuvant or neoadjuvant chemotherapy and progressed within 12 months of treatment with a platinum-containing adjuvant or neoadjuvant regimen.

In some aspects, the subject received one cycle of a platinum-containing chemotherapy regimen but discontinued because of a Grade 4 hematologic toxicity or a Grade 3-4 non-hematologic toxicity.

In some aspects, the subject received no more than two prior lines of treatment for the locally advanced or metastatic UC.

In some aspects, the subject has not received prior treatment with a T-cell co-stimulating therapy or a checkpoint inhibitor.

In some aspects, the anti-latent TGF-beta 1 antibody is administered to the subject in combination with one or more additional therapeutic agents.

In some aspects, the one or more additional therapeutic agents comprises a checkpoint inhibitor.

In some aspects, the checkpoint inhibitor comprises a PD-1 axis binding antagonist or a CTLA4 antagonist.

In some aspects, the checkpoint inhibitor comprises a PD-1 axis binding antagonist.

In some aspects, the PD-1 axis binding antagonist comprises a PD-L1 binding antagonist, a PD-1 binding antagonist, or a PD-L2 binding antagonist.

In some aspects, the PD-1 axis binding antagonist comprises a PD-L1 binding antagonist.

In some aspects, the PD-L1 binding antagonist comprises an anti-PD-L1 antibody.

In some aspects, the anti-PD-L1 antibody comprises atezolizumab, durvalumab, avelumab, or MDX-1105.

In some aspects, the anti-PD-L1 antibody comprises atezolizumab.

In some aspects, the atezolizumab is administered to the subject in a dosing regimen comprising one or more dosing cycles.

In some aspects, the one or more dosing cycles comprise 21-day dosing cycles.

In some aspects, the atezolizumab is administered to the subject on Day 1 of each 21-day dosing cycle.

In some aspects, the atezolizumab is administered to the subject at a dose of 1200 mg.

In some aspects, the one or more dosing cycles comprise 14-day dosing cycles or 28-day dosing cycles.

In some aspects, the one or more dosing cycles comprise 14-day dosing cycles, and the atezolizumab is administered to the subject at a dose of 840 mg.

In some aspects, the atezolizumab is administered to the subject on Day 1 of each 14-day dosing cycle.

In some aspects, the one or more dosing cycles comprise 28-day dosing cycles, and the atezolizumab is administered to the subject at a dose of 1680 mg.

In some aspects, the atezolizumab is administered to the subject on Day 1 of each 28-day dosing cycle.

In some aspects, the atezolizumab is administered to the subject intravenously.

In some aspects, the atezolizumab is administered to the subject intravenously by infusion.

In some aspects, the PD-1 axis binding antagonist comprises a PD-1 binding antagonist.

In some aspects, the PD-1 binding antagonist comprises an anti-PD-1 antibody.

In some aspects, the anti-PD-1 antibody comprises nivolumab, pembrolizumab, MEDI-0680, spartalizumab, cemiplimab, prolgolimab, camrelizumab, sintilimab, tislelizumab, toripalimab, dostarlimab, retifanlimab, sasanlimab, penpulimab, zimberelimab, balstilimab, genolimzumab, cetrelimab, or budigalimab.

In some aspects, the anti-PD-1 antibody comprises nivolumab.

In some aspects, the nivolumab is administered to the subject in a dosing regimen comprising one or more dosing cycles.

In some aspects, the one or more dosing cycles comprise 21-day dosing cycles.

In some aspects, the nivolumab is administered to the subject on Day 1 of each 21-day dosing cycle.

In some aspects, the nivolumab is administered to the subject at a dose of 360 mg.

In some aspects, the nivolumab is administered to the subject intravenously.

In some aspects, the nivolumab is administered to the subject intravenously by infusion.

In some aspects, the one or more additional therapeutic agents is selected from a chemotherapeutic agent, an immunotherapy agent, a radiation therapy agent, an anti-angiogenic agent, and any combination thereof.

In some aspects, the one or more additional therapeutic agents comprise one or more chemotherapeutic agents.

In some aspects, the one or more chemotherapeutic agents comprise a platinum-based chemotherapeutic agent, an antimetabolite, a cytotoxic agent, a growth inhibitory agent, a taxane, a folate analog, or any combination thereof.

In some aspects, the platinum-based chemotherapeutic agent comprises oxaliplatin, cisplatin, or carboplatin.

In some aspects, the platinum-based chemotherapeutic agent comprises oxaliplatin.

In some aspects, the oxaliplatin is administered to the subject in a dosing regimen comprising one or more dosing cycles.

In some aspects, the one or more dosing cycles comprise 21-day dosing cycles.

In some aspects, the oxaliplatin is administered to the subject on Day 1 of each 21-day dosing cycle.

In some aspects, the oxaliplatin is administered to the subject at a dose of 130 mg/m².

In some aspects, the oxaliplatin is administered to the subject intravenously.

In some aspects, the antimetabolite comprises capecitabine, gemcitabine, 5-fluorouracil, or tegafur.

In some aspects, the antimetabolite comprises capecitabine.

In some aspects, the capecitabine is administered to the subject in a dosing regimen comprising one or more dosing cycles.

In some aspects, the one or more dosing cycles comprise 21-day dosing cycles.

In some aspects, the capecitabine is administered to the subject on Days 1-14 of each 21-day dosing cycle.

In some aspects, the capecitabine is administered to the subject at a dose of 1000 mg/m² twice daily.

In some aspects, the capecitabine is administered to the subject orally.

In some aspects, the antimetabolite comprises gemcitabine.

In some aspects, the gemcitabine is administered to the subject in a dosing regimen comprising one or more dosing cycles.

In some aspects, the one or more dosing cycles comprise 28-day dosing cycles.

In some aspects, the gemcitabine is administered to the subject on Days 1, 8, and 15 of each 28-day dosing cycle.

In some aspects, the gemcitabine is administered to the subject at a dose of 1000 mg/m² twice daily.

In some aspects, the gemcitabine is administered to the subject intravenously.

In some aspects, the antimetabolite comprises tegafur.

In some aspects, the antimetabolite comprises S-1 (tegafur-gimeracil-oteracil potassium).

In some aspects, the S-1 is administered to the subject in a dosing regimen comprising one or more dosing cycles.

In some aspects, the one or more dosing cycles comprise 21-day dosing cycles.

In some aspects, the S-1 is administered to the subject on Days 1-14 of each 21-day dosing cycle.

In some aspects, the S-1 is administered to the subject at a dose of 40 mg/m² twice daily.

In some aspects, the S-1 is administered to the subject orally.

In some aspects, the taxane comprises nab-paclitaxel or paclitaxel.

In some aspects, the taxane comprises nab-paclitaxel.

In some aspects, the nab-paclitaxel is administered to the subject in a dosing regimen comprising one or more dosing cycles.

In some aspects, the one or more dosing cycles comprise 28-day dosing cycles.

In some aspects, the nab-paclitaxel is administered to the subject on Days 1, 8, and 15 of each 28-day dosing cycle.

In some aspects, the nab-paclitaxel is administered to the subject at a dose of 125 mg/m².

In some aspects, the nab-paclitaxel is administered to the subject intravenously.

In some aspects, the folate analog comprises leucovorin.

In another aspect, the invention provides a method of treating a subject having a locally advanced, recurrent, or metastatic NSCLC, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 1200 mg intravenously on Day 1 of each 21-day dosing cycle.

In another aspect, the invention provides an anti-latent TGF-beta 1 for use in treatment of a subject having a locally advanced, recurrent, or metastatic NSCLC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some aspects, the NSCLC is histologically or cytologically confirmed metastatic nonsquamous NSCLC or metastatic squamous NSCLC.

In some aspects, the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

In some aspects, the subject has previously received a combination therapy comprising a platinum-containing chemotherapy regimen and a PD-1 axis binding antagonist.

In some aspects, the subject has previously received a platinum-containing chemotherapy regimen and a PD-1 axis binding antagonist as individual regimens.

In some aspects, the subject had disease progression or recurrence within 6 months of definitive therapy for locally advanced NSCLC.

In some aspects, a tumor sample from the subject has been determined to have a detectable level of PD-L1 expression.

In another aspect, the invention provides a method of treating a subject having a locally advanced, recurrent, or metastatic gastric cancer, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) nivolumab at a dosage of 360 mg intravenously on Day 1 of each 21-day dosing cycle; (iii) (a) capecitabine at a dosage of 1000 mg/m² orally twice daily on Days 1-14 of each 21-day dosing cycle, or (b) S-1 at a dosage of 40 mg/m² orally twice daily on Days 1-14 of each 21-day dosing cycle; and (iv) oxaliplatin at a dosage of 130 mg/m² intravenously on Day 1 of each 21-day dosing cycle.

In another aspect, the invention provides an anti-latent TGF-beta 1 for use in treatment of a subject having a locally advanced, recurrent, or metastatic gastric cancer, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some aspects, the subject has unresectable locally advanced or metastatic gastric cancer that is histologically confirmed to be adenocarcinoma.

In some aspects, the gastric cancer comprises esophagogastric junction cancer.

In some aspects, the gastric cancer is HER2-negative gastric cancer.

In some aspects, the subject is previously untreated for gastric cancer and/or the subject has not been previously treated with a checkpoint inhibitor.

In another aspect, the invention provides a method of treating a subject having a locally advanced, recurrent, or metastatic PDAC, the method comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle.

In another aspect, the invention provides an anti-latent TGF-beta 1 for use in treatment of a subject having a locally advanced, recurrent, or metastatic PDAC, the treatment comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle.

In some aspects, the subject has histologically or cytologically confirmed metastatic PDAC.

In some aspects, the subject is previously untreated for the PDAC and/or the subject has not been previously treated with a checkpoint inhibitor.

In another aspect, the invention provides a method of treating a subject having a locally advanced, recurrent, or metastatic UC, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 1200 mg intravenously on Day 1 of each 21-day dosing cycle.

In another aspect, the invention provides an anti-latent TGF-beta 1 for use in treatment of a subject having a locally advanced, recurrent, or metastatic UC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 1200 mg intravenously on Day 1 of each 21-day dosing cycle.

In some aspects, the subject has histologically documented, locally advanced (T4b, any N; or any T, N2-N3) UC, or metastatic UC (M1, Stage 4).

In some aspects, the subject is previously untreated for UC.

In some aspects, the subject is ineligible for cisplatin-containing chemotherapy.

In some aspects, the subject is ineligible for cisplatin-containing chemotherapy as defined by any one of the following criteria: (i) impaired renal function in terms of glomerular filtration rate (GFR) of ≥30 mL/min but <60 mL/min as assessed by direct measurement or by calculation from serum or plasma creatinine; (ii) hearing loss of 25 dB at two contiguous frequencies as measured by audiometry; (iii) Grade 2 peripheral neuropathy; or (iv) ECOG Performance Status of 2.

In some aspects, the subject has previously received at least one platinum-containing chemotherapy regimen.

In some aspects, the subject had disease progression during or following treatment with at least one platinum-containing chemotherapy regimen.

In some aspects, the at least one platinum-containing chemotherapy regimen comprised (i) gemcitabine and cisplatin or carboplatin or (ii) methotrexate, vinblastine, doxorubicin, and cisplatin.

In some aspects, the subject received prior adjuvant or neoadjuvant chemotherapy and progressed within 12 months of treatment with a platinum-containing adjuvant or neoadjuvant regimen.

In some aspects, the subject received one cycle of a platinum-containing chemotherapy regimen but discontinued because of a Grade 4 hematologic toxicity or a Grade 3-4 non-hematologic toxicity.

In some aspects, the subject received no more than two prior regimens of treatment for the locally advanced or metastatic UC.

In some aspects, the subject has not received prior treatment with a T-cell co-stimulating therapy or a checkpoint inhibitor.

In another aspect, the invention provides a method of treating a subject having a metastatic non-squamous NSCLC or a metastatic squamous NSCLC, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

In another aspect, the invention provides an anti-latent TGF-beta 1 for use in treatment of a subject having a metastatic non-squamous NSCLC or a metastatic squamous NSCLC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

In another aspect, the invention provides a method of treating a subject having a locally advanced, unresectable, or metastatic HER2-negative gastric cancer, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) nivolumab at a dosage of 360 mg intravenously on Day 1 of each 21-day dosing cycle; (iii) (a) capecitabine at a dosage of 1000 mg/m² orally twice daily on Days 1-14 of each 21-day dosing cycle, or (b) S-1 at a dosage of 40 mg/m² orally twice daily on Days 1-14 of each 21-day dosing cycle; and (iv) oxaliplatin at a dosage of 130 mg/m² intravenously on Day 1 of each 21-day dosing cycle, wherein the subject is previously untreated for the locally advanced, unresectable, or metastatic HER2-negative gastric cancer.

In another aspect, the invention provides an anti-latent TGF-beta 1 for use in treatment of a subject having a locally advanced, unresectable, or metastatic HER2-negative gastric cancer, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) nivolumab at a dosage of 360 mg intravenously on Day 1 of each 21-day dosing cycle; (iii) (a) capecitabine at a dosage of 1000 mg/m² orally twice daily on Days 1-14 of each 21-day dosing cycle, or (b) S-1 at a dosage of 40 mg/m² orally twice daily on Days 1-14 of each 21-day dosing cycle; and (iv) oxaliplatin at a dosage of 130 mg/m² intravenously on Day 1 of each 21-day dosing cycle, wherein the subject is previously untreated for the locally advanced, unresectable, or metastatic HER2-negative gastric cancer.

In another aspect, the invention provides a method of treating a subject having a metastatic PDAC, the method comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle, wherein the subject is previously untreated for the metastatic PDAC.

In another aspect, the invention provides an anti-latent TGF-beta 1 for use in treatment of a subject having a metastatic PDAC, the treatment comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle, wherein the subject is previously untreated for the metastatic PDAC.

In another aspect, the invention provides a method of treating a subject having a locally advanced or metastatic UC, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein: the subject is previously untreated for the locally advanced or metastatic UC and is ineligible for cisplatin-containing chemotherapy; or the subject has previously received at least one platinum-containing chemotherapy regimen.

In another aspect, the invention provides an anti-latent TGF-beta 1 for use in treatment of a subject having a locally advanced or metastatic UC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein: the subject is previously untreated for the locally advanced or metastatic UC and is ineligible for cisplatin-containing chemotherapy; or the subject has previously received at least one platinum-containing chemotherapy regimen.

In another aspect, the invention provides a method of treating cancer in a subject having a locally advanced or recurrent tumor, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 300 mg to 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively, and (ii) atezolizumab at a dosage of 1200 mg intravenously on Day 1 of each 21-day dosing cycle.

In some aspects, the subject has previously received treatment with atezolizumab at a dosage of 1200 mg intravenously.

In some aspects, the anti-cancer therapy comprises the anti-latent TGF-beta 1 antibody at a dosage of 300 mg. In some aspects, the anti-cancer therapy comprises the anti-latent TGF-beta 1 antibody at a dosage of 600 mg. In some aspects, the anti-cancer therapy comprises the anti-latent TGF-beta 1 antibody at a dosage of 900 mg. In some aspects, the anti-cancer therapy comprises the anti-latent TGF-beta 1 antibody at a dosage of 1200 mg. In some aspects, the anti-cancer therapy comprises the anti-latent TGF-beta 1 antibody at a dosage of 1500 mg. In some aspects, the anti-cancer therapy comprises the anti-latent TGF-beta 1 antibody at a dosage of 1800 mg.

In some aspects, the anti-latent TGF-beta 1 antibody is administered to the subject intravenously.

In some aspects, the anti-latent TGF-beta 1 antibody is administered to the subject intravenously by infusion.

In some aspects, the has been determined to have a detectable level of PD-L1 expression.

In some aspects, the subject's age is 18 years or older.

In some aspects, the anti-cancer therapy is a first-line therapy. In some aspects, the anti-cancer therapy is a second-line therapy or a third-line therapy.

In some aspects, the subject has not been previously treated with a checkpoint inhibitor.

In some aspects, the cancer is NSCLC, gastric cancer, PDAC, UC, GIST, skin cancer, colorectal cancer, OV cancer, renal cancer, or gallbladder cancer. In some aspects, the cancer is GIST, skin cancer, colorectal cancer, OV cancer, renal cancer, or gallbladder cancer.

In another aspect, the invention provides an anti-latent TGF-beta 1 for use in a treatment of cancer in a subject having a locally advanced or recurrent tumor, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 300 mg to 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively, and (ii) atezolizumab at a dosage of 1200 mg intravenously on Day 1 of each 21-day dosing cycle.

In some aspects, the subject is administered the anti-latent TGF-beta 1 antibody until loss of clinical benefit or unacceptable toxicity.

In some aspects, the method comprises between 1 and 20 dosing cycles (e.g., 1 cycle, 2 cycles, 3 cycles, 4 cycles, 5 cycles, 6 cycles, 7 cycles, 8 cycles, 9 cycles, 10, cycles, 11 cycles, 12 cycles, 13 cycles, 14 cycles, 15 cycles, 16 cycles, 17 cycles, 18 cycles, 19 cycles, or 20 cycles).

In some aspects, the anti-latent TGF-beta 1 antibody comprises HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively.

In some aspects, the anti-latent TGF-beta 1 antibody comprises: (a) (i) a heavy chain variable domain (VH) sequence having at least 95% sequence identity to the amino acid sequence of SEQ ID NO: 7, (ii) a light chain variable domain (VL) sequence having at least 95% sequence identity to the amino acid sequence of SEQ ID NO: 8, or (iii) a VH sequence as defined in (i) and a VL sequence as defined in (ii); (b) (i) a VH sequence having at least 95% sequence identity to the amino acid sequence of SEQ ID NO: 31, (ii) a VL sequence having at least 95% sequence identity to the amino acid sequence of SEQ ID NO: 32, or (iii) a VH sequence as defined in (i) and a VL sequence as defined in (ii); (c) (i) a VH sequence having at least 95% sequence identity to the amino acid sequence of SEQ ID NO: 33, (ii) a VL sequence having at least 95% sequence identity to the amino acid sequence of SEQ ID NO: 34, (iii) a VH sequence as defined in (i) and a VL sequence as defined in (ii); or (d) (i) a VH sequence having at least 95% sequence identity to the amino acid sequence of SEQ ID NO: 35, (ii) a VL sequence having at least 95% sequence identity to the amino acid sequence of SEQ ID NO: 36, or (iii) a VH sequence as defined in (i) and a VL sequence as defined in (ii).

In some aspects, the anti-latent TGF-beta 1 antibody comprises: (a) a VH sequence of SEQ ID NO: 7 and a VL sequence of SEQ ID NO: 8; (b) a VH sequence of SEQ ID NO: 31 and a VL sequence of SEQ ID NO: 32; (c) a VH sequence of SEQ ID NO: 33 and a VL sequence of SEQ ID NO: 34; or (d) a VH sequence of SEQ ID NO: 35 and a VL sequence of SEQ ID NO: 36.

In some aspects, the anti-latent TGF-beta 1 antibody comprises a VH sequence of SEQ ID NO: 7 and a VL sequence of SEQ ID NO: 8.

In some aspects, the anti-latent TGF-beta 1 antibody is a chimeric antibody.

In some aspects, the anti-latent TGF-beta 1 antibody is a humanized antibody.

In some aspects, the anti-latent TGF-beta 1 antibody is a full-length antibody.

In some aspects, the anti-latent TGF-beta 1 antibody comprises: (a) a heavy chain comprising the amino acid sequence of SEQ ID NO: 37 and a light chain comprising the amino acid sequence of SEQ ID NO: 38; (b) a heavy chain comprising the amino acid sequence of SEQ ID NO: 39 and a light chain comprising the amino acid sequence of SEQ ID NO: 40; (c) a heavy chain comprising the amino acid sequence of SEQ ID NO: 41 and a light chain comprising the amino acid sequence of SEQ ID NO: 42; (d) a heavy chain comprising the amino acid sequence of SEQ ID NO: 43 and a light chain comprising the amino acid sequence of SEQ ID NO: 44; (e) a heavy chain comprising the amino acid sequence of SEQ ID NO: 45 and a light chain comprising the amino acid sequence of SEQ ID NO: 46; (f) a heavy chain comprising the amino acid sequence of SEQ ID NO: 47 and a light chain comprising the amino acid sequence of SEQ ID NO: 48; (g) a heavy chain comprising the amino acid sequence of SEQ ID NO: 49 and a light chain comprising the amino acid sequence of SEQ ID NO: 50; or (h) a heavy chain comprising the amino acid sequence of SEQ ID NO: 51 and a light chain comprising the amino acid sequence of SEQ ID NO: 52.

In some aspects, the anti-latent TGF-beta 1 antibody comprises a modified IgG1 Fc region having reduced effector function compared with a wild-type IgG1 Fc region.

In some aspects, the modified IgG1 Fc region comprises a constant heavy (CH) region comprising one or more of the following substitutions: K214R, L235R, G236R, M428L, N434A, Q438R, and/or S440E (EU numbering).

In some aspects, the CH region comprises the amino acid sequence of SEQ ID NO: 9.

In some aspects, the modified IgG1 Fc region comprises a constant light (CL) domain comprising the amino acid sequence of SEQ ID NO: 10.

In some aspects, the anti-latent TGF-beta 1 antibody comprises a heavy chain comprising the amino acid sequence of SEQ ID NO: 11 and a light chain sequence comprising the amino acid sequence of SEQ ID NO: 12.

In some aspects, the anti-latent TGF-beta 1 antibody is an antibody fragment that binds to latent TGF-beta 1.

In some aspects, the subject is a human.

### BRIEF DESCRIPTION OF THE DRAWINGS

FIG. 1 shows a flow diagram of the study design described in Example 1. 1L=first-line; 2L=second-line; 3L=third-line; GC=gastric cancer; IMC=Internal Monitoring Committee; NSCLC=non-small cell lung cancer; PDAC=pancreatic ductal adenocarcinoma; SOC=standard of care. ^{a}=Efficacy analysis is performed for each cohort after approximately 15 patients enrolled from the safety run-in and expansion stages have completed at least one tumor assessment in a given cohort.

FIG. 2 shows a flow diagram outlining the conditions for continuing treatment beyond disease progression, as discussed in Example 1. ECOG=Eastern Cooperative Oncology Group; RECIST v1.1=Response Evaluation Criteria in Solid Tumors, Version 1.1.

FIG. 3 shows a flow diagram of the study design described in Example 2. 1L=first-line; 2L=second-line; IMC=Internal Monitoring Committee; UC=urothelial carcinoma. ^{a}=Efficacy analysis is performed for each cohort after approximately 15 patients enrolled from the safety run-in and expansion stages have completed at least one tumor assessment in a given cohort.

FIG. 4 shows a diagram outlining the dosing regimen treatments for Cohorts 1-6 described in Example 3. Atezo=atezolizumab.

FIG. 5A shows a time course of plasma SOF10 concentration (μg/mL) in patients from Cohorts 1-5 described in Example 3.

FIG. 5B shows a time course of plasma SOF10 concentration (μg/mL) in patients from Cohort 6 described in Example 3.

FIG. 6 shows a time course of tumor size, relative to baseline, in various cancers following the SOF10 and atezolizumab combinatorial treatment described in Example 3.

FIG. 7A shows a time course of TGF-β1 total plasma concentrations following the first dose of the SOF10 and atezolizumab combinatorial treatment described in Example 3.

FIG. 7B shows a time course of TGF-β1 percent change, relative to baseline, following the first dose of the SOF10 and atezolizumab combinatorial treatment described in Example 3.

### DETAILED DESCRIPTION OF THE INVENTION

The present invention provides therapeutic methods and compositions for treating cancer, for example, a locally advanced, recurrent, or metastatic solid tumor (e.g., non-small cell lung cancer (NSCLC), gastric cancer, pancreatic ductal adenocarcinoma (PDAC), urothelial carcinoma (UC), gastrointestinal stromal tumor (GIST), skin cancer, colorectal cancer, ovarian (OV) cancer, renal cancer, or gallbladder cancer). The present invention is based, at least in part, on the development of dosages and dosing regimens for anti-latent TGF-beta 1 antibodies as described herein designed to provide significant anti-tumor activity. In some examples, and without wishing to be bound by any particular theory, the methods and compositions disclosed herein are expected to target an important resistance mechanism to checkpoint inhibitors and improve efficacy of these agents in cancer patients by altering the immunosuppressive tumor microenvironment.

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some examples, the number of amino acid changes are 10 or less, 9 or less, 8 or less, 7 or less, 6 or less, 5 or less, 4 or less, 3 or less, or 2 or less. In some examples, the VL acceptor human framework is identical in sequence to the VL human immunoglobulin framework sequence or human consensus framework sequence.

The term “antibody” herein is used in the broadest sense and encompasses various antibody structures, including but not limited to monoclonal antibodies, polyclonal antibodies, multispecific antibodies (e.g., bispecific antibodies), and antibody fragments thereof. The term “antibody” also includes any antigen binding molecule which comprises variable heavy chain and/or variable light chain structure(s) of immunoglobulin.

The terms “anti-latent TGF-beta 1 antibody” and “an antibody that can bind to latent TGF-beta 1” refer to an antibody that is capable of binding latent TGF-beta 1 with sufficient binding activity such that the antibody is useful as a diagnostic and/or therapeutic agent in targeting latent TGF-beta 1. In one example, “an antibody that can bind to latent TGF-beta 1” is an antibody that specifically binds to latent TGF-beta 1. In one example, the extent of binding activity of an anti-latent TGF-beta 1 antibody to an unrelated, non-latent TGF-beta 1 protein is less than about 10% of the binding activity of the antibody to latent TGF-beta 1 as measured, e.g., by a radioimmunoassay (RIA). In certain examples, an antibody that can bind to TGF-beta 1 has a dissociation constant (KD) of 1 micromolar or less, 100 nM or less, 10 nM or less, 1 nM or less, 0.1 nM or less, 0.01 nM or less, or 0.001 nM or less (e.g., 10⁻⁸ M or less, e.g., from 10⁻⁸ M to 10⁻¹³ M, e.g., from 10⁻⁹ M to 10⁻¹³ M). In certain examples, an anti-latent TGF-beta 1 antibody binds to an epitope of latent TGF-beta 1 that is conserved among latent TGF-beta 1 from different species. In some examples, the anti-latent TGF-beta 1 antibody is an anti-latent TGF-beta 1 antibody disclosed in WO 2021/039945, which is incorporated herein by reference in its entirety. In some examples, the anti-latent TGF-beta 1 antibody is SOF10.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided in WO 2021/039945.

For the purposes herein, “atezolizumab” is an Fc-engineered, humanized, non-glycosylated IgG1 kappa immunoglobulin that binds PD-L1. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors. Atezolizumab is also described in WHO Drug Information (International Nonproprietary Names for Pharmaceutical Substances (proposed INN)) List 112, Vol. 28, No. 4, 2014, p. 488.

The term “binding activity” refers to the strength of the sum total of noncovalent interactions between one or more binding sites of a molecule (e.g., an antibody) and its binding partner (e.g., an antigen). Herein, “binding activity” is not strictly limited to a 1:1 interaction between members of a binding pair (e.g., antibody and antigen). For example, when the members of a binding pair reflect a monovalent 1:1 interaction, the binding activity is particularly called the intrinsic binding affinity (affinity). When a member of a binding pair is capable of both monovalent binding and multivalent binding, the binding activity is the sum of each binding strength. The binding activity of a molecule X for its partner Y can generally be represented by the dissociation constant (KD) or “binding amount of analyte per unit amount of ligand” (hereinbelow, may be referred to as “binding amount”). Those skilled in the art would understand that, generally, lower value of dissociation constant (KD) means higher binding activity, and higher value of “binding amount of analyte per unit amount of ligand” or “binding amount” means higher binding activity. Binding activity can be measured by common methods known in the art, including those described herein. Specific illustrative and exemplary examples for measuring binding activity are described in the following.

A “binding activity-matured,” “affinity-matured” antigen-binding molecule or antibody, “binding activity-increased (enhanced),” or “affinity-increased (enhanced)” antigen-binding molecule or antibody refers to an antibody with one or more alterations (e.g., substitutions) in one or more hypervariable regions (HVRs), compared to a parent antigen-binding molecule or a parent antibody which does not carry such alterations, such alterations resulting in an improvement in the binding activity of the antigen-binding molecule or antibody for antigen.

The term “cancer” refers to a disease caused by an uncontrolled division of abnormal cells in a part of the body. Aspects of cancer include solid tumor cancers and non-solid tumor cancers. Examples of cancer include but are not limited to, carcinoma, lymphoma, blastoma, sarcoma, and leukemia or lymphoid malignancies. More particular examples of such cancers include, but are not limited to, skin cancer, gallbladder cancer, bladder cancer (e.g., urothelial carcinoma (UC), including metastatic UC (mUC); muscle-invasive bladder cancer (MIBC), and non-muscle-invasive bladder cancer (NMIBC)); kidney or renal cancer (e.g., renal cell carcinoma (RCC)); lung cancer, including small-cell lung cancer, non-small cell lung cancer, adenocarcinoma of the lung, and squamous carcinoma of the lung; cancer of the urinary tract; breast cancer (e.g., HER2+ breast cancer and triple-negative breast cancer (TNBC), which are estrogen receptors (ER−), progesterone receptors (PR−), and HER2 (HER2−) negative); prostate cancer, such as castration-resistant prostate cancer (CRPC); cancer of the peritoneum; hepatocellular cancer; gastric or stomach cancer, including gastrointestinal cancer and gastrointestinal stromal cancer (GIST); pancreatic cancer (e.g., pancreatic ductal adenocarcinoma (PDAC)); glioblastoma; cervical cancer; ovarian cancer (OV); liver cancer (e.g., hepatocellular carcinoma (HCC)); hepatoma; colon cancer; rectal cancer; colorectal cancer; endometrial or uterine carcinoma; salivary gland carcinoma; prostate cancer; vulval cancer; thyroid cancer; hepatic carcinoma; anal carcinoma; penile carcinoma; melanoma, including superficial spreading melanoma, lentigo malignant melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases. In one instance, the cancer is a locally advanced, recurrent, or metastatic solid tumor (e.g., NSCLC, gastric cancer, PDAC, UC, GIST, skin cancer, colorectal cancer, OV cancer, renal cancer, or gallbladder cancer). The cancer may be locally advanced or metastatic. In some instances, the cancer is locally advanced. In other instances, the cancer is metastatic. In some instances, the cancer may be unresectable (e.g., unresectable locally advanced or metastatic cancer). In some instances, the cancer may be recurrent.

As used herein, the term “checkpoint inhibitor” refers to a therapeutic agent that antagonizes an immune checkpoint protein, e.g., by blocking binding of an immune checkpoint protein to one or more of its binding partners. Exemplary checkpoint inhibitors include but are not limited to a PD-1 axis binding antagonist (e.g., a PD-L1 binding antagonist (e.g., an anti-PD-L1 antibody (e.g., atezolizumab)) or a PD-1 binding antagonist (e.g., an anti-PD-1 antibody (e.g., nivolumab)), a CTLA-4 antagonist (e.g., an anti-CTLA-4 antibody (e.g., ipilimumab)), and a LAG-3 antagonist (e.g., an anti-LAG-3 antibody (e.g., relatlimab)).

As used herein, the term “chemotherapeutic agent” refers to a compound useful in the treatment of cancer, such as a locally advanced, recurrent, or metastatic solid tumor (e.g., NSCLC, gastric cancer, PDAC, UC, GIST, skin cancer, colorectal cancer, OV cancer, renal cancer, or gallbladder cancer). Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and (x) pharmaceutically acceptable salts, acids, prodrugs, and derivatives of any of the above.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

Chemotherapeutic agents also include “platinum-based” chemotherapeutic agents, which comprise an organic compound which contains platinum as an integral part of the molecule. Typically, platinum-based chemotherapeutic agents are coordination complexes of platinum. Platinum-based chemotherapeutic agents are sometimes called “platins” in the art. Examples of platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. Platinum-based chemotherapeutic agents (e.g., cisplatin or carboplatin) may be administered in combination with one or more additional chemotherapeutic agents, e.g., an antimetabolite (e.g., gemcitabine).

Herein, an “effective amount” refers to the amount of a therapeutic agent (e.g., an anti-latent TGF-beta 1 antibody) or combination of therapeutic agents (e.g., an anti-latent TGF-beta 1 antibody and one or more additional therapeutic agents, e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents), that achieves a therapeutic result. In some examples, the effective amount of a therapeutic agent or a combination of therapeutic agents is the amount of the agent or of the combination of agents that achieves a clinical endpoint of improved overall response rate (ORR), a complete response (CR), a pathological complete response (pCR), a partial response (PR), improved survival (e.g., disease-free survival (DFS), progression-free survival (PFS) and/or overall survival (OS)), and/or improved duration of response (DOR). Improvement (e.g., in terms of response rate (e.g., ORR, CR, and/or PR), survival (e.g., PFS and/or OS), or DOR) may be relative to a suitable reference treatment, for example, treatment that does not include the anti-latent TGF-beta 1 antibody and/or treatment that does not include the one or more additional therapeutic agents, e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents.

As used herein, “complete response” and “CR” refers to disappearance of all target lesions.

As used herein, “partial response” and “PR” refers to at least a 30% decrease in the sum of the longest diameters (SLD) of target lesions, taking as reference the baseline SLD prior to treatment.

As used herein, “overall response rate,” “objective response rate,” and “ORR” refer interchangeably to the sum of CR rate and PR rate. For example, ORR may be defined the proportion of patients with a CR or PR on two consecutive occasions ≥4 weeks apart, as determined by the investigator according to RECIST v1.1

As used herein, “progression-free survival” and “PFS” refer to the length of time during and after treatment during which the cancer does not get worse. PFS may include the amount of time subjects have experienced a CR or a PR, as well as the amount of time subjects have experienced stable disease. In some examples, PFS is defined as the time from the first anti-TGF-beta 1 antibody treatment day to the first occurrence of disease progression or death from any cause during a study (whichever occurs first), as determined by the investigator according to RECIST v1.1.

As used herein, “overall survival” and “OS” refer to the length of time from either the date of diagnosis or the start of treatment for a disease (e.g., cancer) that the subject is still alive.

As used herein, the term “duration of response” and “DOR” refer to a length of time from documentation of a tumor response until disease progression or death from any cause, whichever occurs first. In some examples, DOR is defined as the time from the first anti-TGF-beta 1 antibody treatment day to the first occurrence of disease progression or death from any cause during a study (whichever occurs first), as determined by the investigator according to RECIST v1.1.

The term “ineligible for cisplatin-containing chemotherapy” means that the subject is not eligible for treatment with a cisplatin-based chemotherapy, either in the attending clinician's judgment or according to standardized criteria for eligibility for platinum-based chemotherapy that are described herein or known in the art. For example, the criteria set forth in Galsky et al. Lancet Oncol. 12(3):211-4, 2011 may be used to determine whether a subject is eligible for cisplatin-based chemotherapy. Galsky et al. describe a consensus definition of subjects with metastatic UC (mUC) in which subjects meeting at least one of the following are considered unfit for cisplatin-based chemotherapy: (i) a World Health Association (WHO) or Eastern Cooperative Oncology Group (ECOG) performance status of 2, or Karnofsky performance status of 60-70%; (ii) creatinine clearance (calculated or measured) less than 1 mL/s; (iii) National Cancer Institute (NCI) Common Terminology Criteria for Adverse Events (CTCAE) v4.0 Grade>2 audiometric hearing loss; (iv) CTCAE v.4.0 Grade ≥2 peripheral neuropathy; and/or New York Heart Association (NYHA) class III heart failure. In one example, a subject is ineligible for cisplatin-based chemotherapy if they have one or more of the following: impaired renal function (e.g., glomerular filtration rate (GFR) >30 but <60 mL/min); GFR may be assessed by direct measurement (i.e., creatinine clearance or ethyldediaminetetra-acetate) or, if not available, by calculation from serum/plasma creatinine (e.g., Cockcroft-Gault formula)); hearing loss (e.g., National Cancer Institute (NCI) Common Terminology Criteria for Adverse Events (CTCAE) v4.0 Grade ≥2 audiometric hearing loss of 25 decibels at two contiguous frequencies); peripheral neuropathy (e.g., NCI CTCAE v4.0 Grade ≥2 peripheral neuropathy (i.e., sensory alteration or paresthesia, including tingling)); and/or ECOG performance status assessment (see Oken et al. Am. J. Clin. Oncol. 5:649-655, 1982) (e.g., an ECOG performance status of 2).

A “taxane” as used herein is an agent (e.g., a diterpene) which may bind to tubulin, promoting microtubule assembly and stabilization and/or prevent microtubule depolymerization. Exemplary taxanes include, but are not limited to, paclitaxel (i.e., TAXOL®, CAS #33069-62-4), docetaxel (i.e., TAXOTERE®, CAS #114977-28-5), larotaxel, cabazitaxel, milataxel, tesetaxel, and/or orataxel. Taxanes included herein also include taxoid 10-deacetylbaccatin III and/or derivatives thereof. In some examples, the taxane is an albumin-coated nanoparticle (e.g., nano-albumin bound (nab)-paclitaxel, i.e., ABRAXANE® and/or nab-docetaxel, ABI-008). In some examples, the taxane is nab-paclitaxel (ABRAXANE®). In some examples, the taxane is formulated in CREMAPHOR® (e.g., TAXOL®) and/or in TWEEN® such as polysorbate 80 (e.g., TAXOTERE®). In some examples, the taxane is liposome-encapsulated taxane. In some examples, the taxane is a prodrug form and/or conjugated form of taxane (e.g., DHA covalently conjugated to paclitaxel, paclitaxel poliglumex, and/or linoleyl carbonate-paclitaxel). In some examples, the paclitaxel is formulated with substantially no surfactant (e.g., in the absence of CREMAPHOR® and/or TWEEN®, such as TOCOSOL® paclitaxel).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

As used herein, “in combination with” refers to administration of one treatment modality in addition to another treatment modality, for example, an anti-cancer therapy that includes administration of an anti-latent TGF-beta 1 antibody and one or more additional therapeutic agents, e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents. As such, “in combination with” refers to administration of one treatment modality before, during, or after administration of the other treatment modality to the subject.

A drug that is administered “concurrently” with one or more other drugs is administered during the same treatment cycle, on the same day of treatment, as the one or more other drugs, and, optionally, at the same time as the one or more other drugs. For instance, for cancer therapies given every 3 weeks, the concurrently administered drugs are each administered on day 1 of a 3 week cycle.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain that contains at least a portion of the constant region. The term includes native sequence Fc regions and variant Fc regions. In one example, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one example, for the VL, the subgroup is subgroup kappa I as in Kabat et al., supra. In one example, for the VH, the subgroup is subgroup III as in Kabat et al., supra.

A “humanized” antibody refers to a chimeric antibody comprising amino acid residues from non-human HVRs and amino acid residues from human FRs. In certain examples, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “isolated” antibody is one which has been separated from a component of its natural environment. In some examples, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

“Isolated nucleic acid encoding an anti-latent TGF-beta 1 antibody” or “nucleic acid encoding an anti-latent TGF-beta 1 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation. “Native antibodies” refer to naturally occurring immunoglobulin molecules with varying structures.

For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (kappa) and lambda (lambda), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-88, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The terms “programmed death ligand 1” and “PD-L1” refer herein to native sequence human PD-L1 polypeptide. Native sequence PD-L1 polypeptides are provided under Uniprot Accession No. Q9NZQ7. For example, the native sequence PD-L1 may have the amino acid sequence as set forth in Uniprot Accession No. Q9NZQ7-1 (isoform 1). In another example, the native sequence PD-L1 may have the amino acid sequence as set forth in Uniprot Accession No. Q9NZQ7-2 (isoform 2). In yet another example, the native sequence PD-L1 may have the amino acid sequence as set forth in Uniprot Accession No. Q9NZQ7-3 (isoform 3). PD-L1 is also referred to in the art as “programmed cell death 1 ligand 1,” “PDCD1 LG1,” “CD274,” “B7-H,” and “PDL1.”

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.OD. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “subject” refers to a human subject. For example, the subject may be an adult. The subject may be a patient.

The term “TGF-beta 1,” as used herein, refers to any native TGF-beta 1 from any vertebrate source, including mammals such as primates (e.g., humans) and rodents (e.g., mice and rats), unless otherwise indicated. The term encompasses “full-length” unprocessed TGF-beta 1 as well as any form of TGF-beta 1 that results from processing in the cell. The term also encompasses naturally occurring variants of TGF-beta 1, e.g., splice variants or allelic variants. The amino acid sequence of an exemplary human TGF-beta 1 preproprotein is shown in SEQ ID NO: 53 (NCBI RefSeq: NP 000651.3) and the nucleic acid sequence encoding an exemplary human TGF-beta 1 is shown in SEQ ID NO: 54 (NCBI RefSeq: NM_000660.6). The amino acid sequence of an exemplary mouse TGF-beta 1 preproprotein is shown in SEQ ID NO: 55 (NCBI RefSeq: NP_035707.1) and the nucleic acid sequence encoding an exemplary mouse TGF-beta 1 is shown in SEQ ID NO: 56 (NCBI RefSeq: NM_011577.2). The amino acid sequence of an exemplary cynomolgus monkey TGF-beta 1 preproprotein is shown in SEQ ID NO: 57 (NCBI RefSeq: XP_005589396.1) and the nucleic acid sequence encoding an exemplary cynomolgus monkey TGF-beta 1 is shown in SEQ ID NO: 58 (NCBI RefSeq: XM_005589339.2). The term “TGF-beta 1” encompasses both latent TGF-beta 1 and mature TGF-beta 1.

The term “latent TGF-beta 1,” as used herein, refers to any TGF-beta 1 which forms a latent TGF-beta 1 complex (“cell surface latent TGF-beta 1,” LLC or SLC (see below)) and/or which is incapable of binding to its receptors. Transforming growth factor-beta 1 (TGF-beta 1) is a member of TGF-beta, which is a member of TGF-beta superfamily. Like other members of TGF-beta superfamily, TGF-beta is synthesized as a precursor protein, which forms a homodimer that interacts with its latency-associated peptide (LAP) and a latent TGF-beta-binding protein (LTBP), forming a larger complex called the large latent complex (LLC). The amino acid sequence of an exemplary latent human TGF-beta 1 (TGF-beta homodimer and its LAP) is amino acids 30-390 of SEQ ID NO: 53. The amino acid sequence of and exemplary mouse latent TGF-beta 1 (TGF-beta homodimer and its LAP) is amino acids 30-390 of SEQ ID NO: 55. The amino acid sequence of an exemplary latent cynomolgus monkey TGF-beta 1 (TGF-beta homodimer and its LAP) is amino acids 30-390 of SEQ ID NO: 57.

A complex formed from the TGF-beta homodimer and its LAP is called Small Latent Complex (SLC). This latent complex keeps TGF-beta in an inactive form, which is incapable of binding to its receptors. SLC may be covalently linked to an additional protein, latent TGF-beta binding protein (LTBP), forming the large latent complex (LLC). There are four different LTBP isoforms known, LTBP-1, LTBP-2, LTBP-3 and LTBP-4. It has been reported that LTBP-1, LTBP-3 and LTBP-4 bind to SLC (see, e.g., Rifkin et al., J Biol Chem. 2005 Mar. 4; 280(9):7409-12). SLC may also be covalently linked to other additional proteins, such as glycoprotein A repetitions predominant (GARP) or leucine-rich repeat-containing protein 33 (LRRC33). GARP and LRRC have a transmembrane domain and associate with LAP on the cell surface (see, e.g., Wang et al., Mol Biol Cell. 2012 March; 23(6):1129-39). As to LLCs, it is reported that LLCs associate covalently with the extracellular matrix (ECM) via the N-termini of the LTBPs (see, e.g., Saharinen et al., Cytokine Growth Factor Rev. 1999 June; 10(2):99-117.). In some examples, latent TGF-beta 1 associated with the ECM on a cell surface is referred to as “cell surface latent TGF-beta 1.”

The term “active TGF-beta 1,” “mature TGF-beta 1,” or “active mature TGF-beta 1,” as used herein, refers to any TGF-beta 1 homodimer which does not form a latent TGF-beta 1 complex (LLC or SLC) and which is capable of binding to its receptors. The TGF-beta 1 activation process involves the release of the LLC from the ECM, followed by further proteolysis of LAP to release active TGF-beta to its receptors. A wide range of proteases including plasmin (PLN), prekallikrein (PLK), matrix metalloproteinase (MMP) 2, MMP9, MMP13, MMP14, thrombin, tryptase and calpain are known to cleave latent TGF-beta and release active TGF-beta. These proteases may be collectively called “(latent) TGF-beta-cleaving proteases” or “(latent) TGF-beta 1-cleaving proteases” in the context of the present invention. In addition to proteases, thrombospondin 1 (TSP-1), Neuropilin-1 (Nrp1), ADAMSTS1 and F-spondin activate latent TGF-beta. Alternatively, upon mechanical stretch, integrins (preferably integrin alpha V beta 8 and/or integrin alpha V beta 6) can activate TGF-beta by binding to the RGD motif present in LAP and inducing the release of mature TGF-beta from its latent complex form.

As used herein, “treating” comprises effective cancer treatment with an effective amount of a therapeutic agent (e.g., an anti-latent TGF-beta 1 antibody) or combination of therapeutic agents (e.g., an anti-latent TGF-beta 1 antibody and one or more additional therapeutic agents, e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents). Treating herein includes, inter alia, adjuvant therapy, neoadjuvant therapy, non-metastatic cancer therapy (e.g., locally advanced cancer therapy), and metastatic cancer therapy. The treatment may be first-line treatment (e.g., the subject may be previously untreated or not have received prior systemic therapy), or second line or later (e.g., third line, fourth line, fifth line, or later) treatment.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

As used herein, the term “unresectable” refers to a cancer for which surgical resection is not possible or cannot be safely performed.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

### II. Therapeutic Methods and Compositions for Cancer

Provided herein are therapeutic methods and compositions for use in treating cancer (e.g., a locally advanced, recurrent, or metastatic solid tumor) in a subject, for example, that include administering to the subject an anti-cancer therapy that includes an anti-latent TGF-beta 1 antibody.

In one example, provided herein is a method of treating a subject having a cancer (e.g., a locally advanced, recurrent, or metastatic solid tumor), the method comprising administering to the subject an anti-cancer therapy comprising an anti-latent TGF-beta 1 antibody at a dosage of 1800 mg.

In another example, provided herein is an anti-latent TGF-beta 1 for use in treatment of a subject having a cancer (e.g., a locally advanced, recurrent, or metastatic solid tumor), the treatment comprising administering to the subject an anti-cancer therapy comprising an anti-latent TGF-beta 1 antibody at a dosage of 1800 mg.

In another example, provided herein is the use of an anti-latent TGF-beta 1 in the manufacture of a medicament for treatment of a subject having a cancer (e.g., a locally advanced, recurrent, or metastatic solid tumor), the treatment comprising administering to the subject an anti-cancer therapy comprising an anti-latent TGF-beta 1 antibody at a dosage of 1800 mg.

In some examples, the anti-latent TGF-beta 1 antibody is administered to the subject in a dosing regimen comprising one or more 21-day dosing cycles. For example, the anti-latent TGF-beta 1 antibody may be administered to the subject on Day 1 of each 21-day dosing cycle.

In another example, provided herein is a method of treating a subject having a cancer (e.g., a locally advanced, recurrent, or metastatic solid tumor), the method comprising administering to the subject an anti-cancer therapy comprising an anti-latent TGF-beta 1 antibody at a dosage of 1800 mg every three weeks (Q3W).

In another example, provided herein is an anti-latent TGF-beta 1 for use in treatment of a subject having a cancer (e.g., a locally advanced, recurrent, or metastatic solid tumor), the treatment comprising administering to the subject an anti-cancer therapy comprising an anti-latent TGF-beta 1 antibody at a dosage of 1800 mg every three weeks (Q3W).

In another example, provided herein is the use of an anti-latent TGF-beta 1 in the manufacture of a medicament for treatment of a subject having a cancer (e.g., a locally advanced, recurrent, or metastatic solid tumor), the treatment comprising administering to the subject an anti-cancer therapy comprising an anti-latent TGF-beta 1 antibody at a dosage of 1800 mg every three weeks (Q3W).

In another example, provided herein is a method of treating a subject having a cancer (e.g., a locally advanced, recurrent, or metastatic solid tumor), the method comprising administering to the subject an anti-cancer therapy comprising an anti-latent TGF-beta 1 antibody at a dosage of 1200 mg.

In another example, provided herein is an anti-latent TGF-beta 1 for use in treatment of a subject having a cancer (e.g., a locally advanced, recurrent, or metastatic solid tumor), the treatment comprising administering to the subject an anti-cancer therapy comprising an anti-latent TGF-beta 1 antibody at a dosage of 1200 mg.

In another example, provided herein is the use of an anti-latent TGF-beta 1 in the manufacture of a medicament for treatment of a subject having a cancer (e.g., a locally advanced, recurrent, or metastatic solid tumor), the treatment comprising administering to the subject an anti-cancer therapy comprising an anti-latent TGF-beta 1 antibody at a dosage of 1200 mg.

In some examples, the anti-latent TGF-beta 1 antibody is administered to the subject in a dosing regimen comprising one or more 28-day dosing cycles. For example, the anti-latent TGF-beta 1 antibody may be administered to the subject on Days 1 and 15 of each 28-day dosing cycle.

In another aspect, the invention provides a method of treating a subject having a cancer (e.g., a locally advanced, recurrent, or metastatic solid tumor), the method comprising administering to the subject an anti-cancer therapy comprising an anti-latent TGF-beta 1 antibody at a dosage of 1200 mg every two weeks (Q2W).

In another aspect, the invention provides an anti-latent TGF-beta 1 for use in treatment of a subject having a cancer (e.g., a locally advanced, recurrent, or metastatic solid tumor), the treatment comprising administering to the subject an anti-cancer therapy comprising an anti-latent TGF-beta 1 antibody at a dosage of 1200 mg every two weeks (Q2W).

In another aspect, the invention provides the use of an anti-latent TGF-beta 1 in the manufacture of a medicament for treatment of a subject having a cancer (e.g., a locally advanced, recurrent, or metastatic solid tumor), the treatment comprising administering to the subject an anti-cancer therapy comprising an anti-latent TGF-beta 1 antibody at a dosage of 1200 mg every two weeks (Q2W).

Any suitable cancer may be treated. In some examples, the cancer is a locally advanced, recurrent, or metastatic solid tumor. In some examples, the cancer is NSCLC, gastric cancer, PDAC, or UC.

Any suitable anti-latent TGF-beta 1 antibody may be administered to the subject, including any anti-latent TGF-beta 1 antibody disclosed herein or in WO 2021/039945 or in Section III below.

In some examples, the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively. In particular examples, the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively.

In some examples, the cancer is a locally advanced, recurrent, or metastatic solid tumor.

For example, provided herein is a method of treating a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the method comprising administering to the subject an anti-cancer therapy comprising an anti-latent TGF-beta 1 antibody at a dosage of 1800 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is an anti-latent TGF-beta 1 for use in treatment of a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the treatment comprising administering to the subject an anti-cancer therapy comprising an anti-latent TGF-beta 1 antibody at a dosage of 1800 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is the use of an anti-latent TGF-beta 1 in the manufacture of a medicament for treatment of a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the treatment comprising administering to the subject an anti-cancer therapy comprising an anti-latent TGF-beta 1 antibody at a dosage of 1800 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some examples, the anti-latent TGF-beta 1 antibody is administered to the subject in a dosing regimen comprising one or more 21-day dosing cycles. For example, the anti-latent TGF-beta 1 antibody may be administered to the subject on Day 1 of each 21-day dosing cycle.

In another example, provided herein is a method of treating a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the method comprising administering to the subject an anti-cancer therapy comprising an anti-latent TGF-beta 1 antibody at a dosage of 1800 mg every three weeks (Q3W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is an anti-latent TGF-beta 1 for use in treatment of a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the treatment comprising administering to the subject an anti-cancer therapy comprising an anti-latent TGF-beta 1 antibody at a dosage of 1800 mg every three weeks (Q3W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is the use of an anti-latent TGF-beta 1 in the manufacture of a medicament for treatment of a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the treatment comprising administering to the subject an anti-cancer therapy comprising an anti-latent TGF-beta 1 antibody at a dosage of 1800 mg every three weeks (Q3W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is a method of treating a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the method comprising administering to the subject an anti-cancer therapy comprising an anti-latent TGF-beta 1 antibody at a dosage of 1200 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is an anti-latent TGF-beta 1 for use in treatment of a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the treatment comprising administering to the subject an anti-cancer therapy comprising an anti-latent TGF-beta 1 antibody at a dosage of 1200 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is the use of an anti-latent TGF-beta 1 in the manufacture of a medicament for treatment of a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the treatment comprising administering to the subject an anti-cancer therapy comprising an anti-latent TGF-beta 1 antibody at a dosage of 1200 mg, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some examples, the anti-latent TGF-beta 1 antibody is administered to the subject in a dosing regimen comprising one or more 28-day dosing cycles. For example, the anti-latent TGF-beta 1 antibody may be administered to the subject on Days 1 and 15 of each 28-day dosing cycle.

In another example, provided herein is a method of treating a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the method comprising administering to the subject an anti-cancer therapy comprising an anti-latent TGF-beta 1 antibody at a dosage of 1200 mg every two weeks (Q2W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is an anti-latent TGF-beta 1 for use in treatment of a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the treatment comprising administering to the subject an anti-cancer therapy comprising an anti-latent TGF-beta 1 antibody at a dosage of 1200 mg every two weeks (Q2W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is the use of an anti-latent TGF-beta 1 in the manufacture of a medicament for treatment of a subject having a locally advanced, recurrent, or metastatic solid tumor (e.g., NSCLC, gastric cancer, PDAC, UC, GIST, skin cancer, colorectal cancer, OV cancer, renal cancer, or gallbladder cancer), the treatment comprising administering to the subject an anti-cancer therapy comprising an anti-latent TGF-beta 1 antibody at a dosage of 1200 mg every two weeks (Q2W), wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

The anti-latent TGF-beta 1 antibody may be administered by any suitable route. In some examples, the anti-latent TGF-beta 1 antibody is administered to the subject intravenously. In some examples, the anti-latent TGF-beta 1 antibody is administered to the subject intravenously by infusion.

In some examples, a tumor sample from the subject has been determined to have a detectable level of PD-L1 expression. Any suitable approach to detect PD-L1 expression may be used, including any approach described herein (see, e.g., Section V below).

In some examples, the subject's age is 18 years or older. The subject may be an adult.

The anti-cancer therapy may represent any suitable line of treatment. In some examples, the anti-cancer therapy is a first-line therapy. In other examples, the anti-cancer therapy is a second-line therapy or a third-line therapy.

In some examples, the anti-cancer therapy is a first-line therapy. For example, the patient may be previously untreated. In some examples, the subject has not been previously treated with a checkpoint inhibitor.

In some examples, the solid tumor is metastatic. In other examples, the solid tumor is locally advanced. In yet other examples, the solid tumor is recurrent.

Any suitable solid tumor may be treated. In some examples, the locally advanced, recurrent, or metastatic solid tumor is NSCLC, gastric cancer, PDAC, or UC.

In some examples, the locally advanced, recurrent, or metastatic solid tumor is NSCLC. For example, the NSCLC may be histologically or cytologically confirmed metastatic nonsquamous NSCLC or metastatic squamous NSCLC. In some examples, the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy. In some examples, the subject has previously received a combination therapy comprising a platinum-containing chemotherapy regimen and a PD-1 axis binding antagonist. In some examples, the subject has previously received a platinum-containing chemotherapy regimen and a PD-1 axis binding antagonist as individual regimens. In some examples, the subject had disease progression or recurrence within 6 months of definitive therapy for locally advanced NSCLC. In some examples, a tumor sample from the subject has been determined to have a detectable level of PD-L1 expression.

In some examples, the locally advanced, recurrent, or metastatic solid tumor is gastric cancer. In some examples, the subject has unresectable locally advanced or metastatic gastric cancer that is histologically confirmed to be adenocarcinoma. In some examples, the gastric cancer comprises esophagogastric junction cancer. In some examples, the gastric cancer is HER2-negative gastric cancer.

In some examples, the subject is previously untreated for gastric cancer and/or the subject has not been previously treated with a checkpoint inhibitor.

In some examples, the locally advanced, recurrent, or metastatic solid tumor is PDAC. In some examples, the subject has histologically or cytologically confirmed metastatic PDAC. In some examples, the subject is previously untreated for the PDAC and/or the subject has not been previously treated with a checkpoint inhibitor.

In some examples, the locally advanced, recurrent, or metastatic solid tumor is UC. In some examples, the subject has histologically documented, locally advanced (T4b, any N; or any T, N2-N3) UC, or metastatic UC (M1, Stage 4). In some examples, the subject is previously untreated for UC. In some examples, the subject is ineligible for cisplatin-containing chemotherapy. In some examples, the subject is ineligible for cisplatin-containing chemotherapy as defined by any one of the following criteria: (i) impaired renal function in terms of glomerular filtration rate (GFR) of ≥30 mL/min but <60 mL/min as assessed by direct measurement or by calculation from serum or plasma creatinine; (ii) hearing loss of 25 dB at two contiguous frequencies as measured by audiometry; (iii) Grade 2 peripheral neuropathy; or (iv) Eastern Cooperative Oncology Group (ECOG) Performance Status of 2. In some examples, the subject has previously received at least one platinum-containing chemotherapy regimen. In some examples, the subject had disease progression during or following treatment with at least one platinum-containing chemotherapy regimen. In some examples, the at least one platinum-containing chemotherapy regimen comprised (i) gemcitabine and cisplatin or carboplatin or (ii) methotrexate, vinblastine, doxorubicin, and cisplatin. In some examples, the subject received prior adjuvant or neoadjuvant chemotherapy and progressed within 12 months of treatment with a platinum-containing adjuvant or neoadjuvant regimen. In some examples, the subject received one cycle of a platinum-containing chemotherapy regimen but discontinued because of a Grade 4 hematologic toxicity or a Grade 3-4 non-hematologic toxicity. In some examples, the subject received no more than two prior lines of treatment for the locally advanced or metastatic UC. In some examples, the subject has not received prior treatment with a T-cell co-stimulating therapy or a checkpoint inhibitor.

In some examples, the anti-latent TGF-beta 1 antibody comprises: (a) (i) a heavy chain variable domain (VH) sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 7, (ii) a light chain variable domain (VL) sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 8, or (iii) a VH sequence as defined in (i) and a VL sequence as defined in (ii); (b) (i) a VH sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 31, (ii) a VL sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 32, or (iii) a VH sequence as defined in (i) and a VL sequence as defined in (ii); (c) (i) a VH sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 33, (ii) a VL sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 34, (iii) a VH sequence as defined in (i) and a VL sequence as defined in (ii); or (d) (i) a VH sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 35, (ii) a VL sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 36, or (iii) a VH sequence as defined in (i) and a VL sequence as defined in (ii).

In some examples, the anti-latent TGF-beta 1 antibody comprises: (a) a VH sequence of SEQ ID NO: 7 and a VL sequence of SEQ ID NO: 8; (b) a VH sequence of SEQ ID NO: 31 and a VL sequence of SEQ ID NO: 32; (c) a VH sequence of SEQ ID NO: 33 and a VL sequence of SEQ ID NO: 34; or (d) a VH sequence of SEQ ID NO: 35 and a VL sequence of SEQ ID NO: 36.

In a particular example, the anti-latent TGF-beta 1 antibody comprises a VH sequence of SEQ ID NO: 7 and a VL sequence of SEQ ID NO: 8.

In some examples, the anti-latent TGF-beta 1 antibody is a chimeric antibody.

In some examples, the anti-latent TGF-beta 1 antibody is a humanized antibody.

In some examples, the anti-latent TGF-beta 1 antibody is a full-length antibody.

In some examples, the anti-latent TGF-beta 1 antibody comprises: (a) a heavy chain comprising the amino acid sequence of SEQ ID NO: 37 and a light chain comprising the amino acid sequence of SEQ ID NO: 38; (b) a heavy chain comprising the amino acid sequence of SEQ ID NO: 39 and a light chain comprising the amino acid sequence of SEQ ID NO: 40; (c) a heavy chain comprising the amino acid sequence of SEQ ID NO: 41 and a light chain comprising the amino acid sequence of SEQ ID NO: 42; (d) a heavy chain comprising the amino acid sequence of SEQ ID NO: 43 and a light chain comprising the amino acid sequence of SEQ ID NO: 44; (e) a heavy chain comprising the amino acid sequence of SEQ ID NO: 45 and a light chain comprising the amino acid sequence of SEQ ID NO: 46; (f) a heavy chain comprising the amino acid sequence of SEQ ID NO: 47 and a light chain comprising the amino acid sequence of SEQ ID NO: 48; (g) a heavy chain comprising the amino acid sequence of SEQ ID NO: 49 and a light chain comprising the amino acid sequence of SEQ ID NO: 50; or (h) a heavy chain comprising the amino acid sequence of SEQ ID NO: 51 and a light chain comprising the amino acid sequence of SEQ ID NO: 52.

In some examples, the anti-latent TGF-beta 1 antibody comprises a modified IgG1 Fc region having reduced effector function compared with a wild-type IgG1 Fc region.

In some examples, the modified IgG1 Fc region comprises a constant heavy (CH) region comprising one or more of the following substitutions: K214R, L235R, G236R, M428L, N434A, Q438R, and/or S440E (EU numbering).

In some examples, the CH region comprises the amino acid sequence of SEQ ID NO: 9.

In some examples, the modified IgG1 Fc region comprises a constant light (CL) domain comprising the amino acid sequence of SEQ ID NO: 10.

In some examples, the anti-latent TGF-beta 1 antibody comprises a heavy chain comprising the amino acid sequence of SEQ ID NO: 11 and a light chain sequence comprising the amino acid sequence of SEQ ID NO: 12.

In some examples, the anti-latent TGF-beta 1 antibody is an antibody fragment that binds to latent TGF-beta 1.

In some examples, the anti-latent TGF-beta 1 antibody is administered to the subject as a monotherapy.

In other examples, the anti-latent TGF-beta 1 antibody is administered to the subject in combination with one or more additional therapeutic agents. Any suitable additional therapeutic agents or combinations of additional therapeutic agents may be administered, including any described herein. In some examples, the one or more additional therapeutic agents comprises a checkpoint inhibitor. In some examples, the checkpoint inhibitor comprises a PD-1 axis binding antagonist or a CTLA4 antagonist. In some examples, the checkpoint inhibitor comprises a PD-1 axis binding antagonist, including any PD-1 axis binding antagonist disclosed herein (see, e.g., Section IV). In some examples, the PD-1 axis binding antagonist comprises a PD-L1 binding antagonist, a PD-1 binding antagonist, or a PD-L2 binding antagonist.

In some examples, the PD-1 axis binding antagonist comprises a PD-L1 binding antagonist. In some examples, the PD-L1 binding antagonist comprises an anti-PD-L1 antibody. Any suitable anti-PD-L1 antibody may be used. In some examples, the anti-PD-L1 antibody comprises atezolizumab, durvalumab, avelumab, or MDX-1105.

In a particular example, the anti-PD-L1 antibody comprises atezolizumab. In some examples, the atezolizumab is administered to the subject in a dosing regimen comprising one or more dosing cycles.

In some examples, the one or more dosing cycles comprise 21-day dosing cycles. In some examples, the atezolizumab is administered to the subject on Day 1 of each 21-day dosing cycle. In some examples, the atezolizumab is administered to the subject at a dose of 1200 mg. In some examples, the one or more dosing cycles comprise 14-day dosing cycles or 28-day dosing cycles. In some examples, the one or more dosing cycles comprise 14-day dosing cycles, and the atezolizumab is administered to the subject at a dose of 840 mg. In some examples, the atezolizumab is administered to the subject on Day 1 of each 14-day dosing cycle. In some examples, the one or more dosing cycles comprise 28-day dosing cycles, and the atezolizumab is administered to the subject at a dose of 1680 mg. In some examples, the atezolizumab is administered to the subject on Day 1 of each 28-day dosing cycle. In some examples, the atezolizumab is administered to the subject intravenously. In some examples, the atezolizumab is administered to the subject intravenously by infusion.

In some instances, atezolizumab is administered to the subject intravenously at a dose of about 840 mg every 2 weeks, about 1200 mg every 3 weeks, or about 1680 mg every 4 weeks.

In other examples, the PD-1 axis binding antagonist comprises a PD-1 binding antagonist. In some examples, the PD-1 binding antagonist comprises an anti-PD-1 antibody. Any suitable anti-PD-1 antibody may be used. In some examples, the anti-PD-1 antibody comprises nivolumab, pembrolizumab, MEDI-0680, spartalizumab, cemiplimab, prolgolimab, camrelizumab, sintilimab, tislelizumab, toripalimab, dostarlimab, retifanlimab, sasanlimab, penpulimab, zimberelimab, balstilimab, genolimzumab, cetrelimab, or budigalimab.

In particular examples, the anti-PD-1 antibody comprises nivolumab. In some examples, the nivolumab is administered to the subject in a dosing regimen comprising one or more dosing cycles. In some examples, the one or more dosing cycles comprise 21-day dosing cycles. In some examples, the nivolumab is administered to the subject on Day 1 of each 21-day dosing cycle. In some examples, the nivolumab is administered to the subject at a dose of 360 mg. In some examples, the nivolumab is administered to the subject intravenously. In some examples, the nivolumab is administered to the subject intravenously by infusion.

In some examples, the one or more additional therapeutic agents is selected from a chemotherapeutic agent, an immunotherapy agent, a radiation therapy agent, an anti-angiogenic agent, and any combination thereof.

In some examples, the one or more additional therapeutic agents includes an agonist directed against an activating co-stimulatory molecule. In some examples, an activating co-stimulatory molecule may include CD40, CD226, CD28, OX40, GITR, CD137, CD27, HVEM, or CD127. In some examples, the one or more additional therapeutic agents includes an agonist antibody that binds to CD40, CD226, CD28, OX40, GITR, CD137, CD27, HVEM, or CD127. In some examples, the one or more additional therapeutic agents includes an antagonist directed against an inhibitory co-stimulatory molecule. In some embodiments, an inhibitory co-stimulatory molecule may include CTLA-4 (also known as CD152), TIM-3, BTLA, VISTA, LAG-3, B7-H3, B7-H4, IDO, TIGIT, MICA/B, or arginase. In some embodiments, the antagonist directed against an inhibitory co-stimulatory molecule is an antagonist antibody that binds to CTLA-4, TIM-3, BTLA, VISTA, LAG-3, B7-H3, B7-H4, IDO, TIGIT, MICA/B, or arginase.

In some examples, the one or more additional therapeutic agents comprise one or more chemotherapeutic agents. Any suitable chemotherapeutic agents may be used, including any chemotherapeutic agents or combinations of chemotherapeutic agents disclosed herein (see, e.g., Section VI below). In some examples, the one or more chemotherapeutic agents comprise a platinum-based chemotherapeutic agent, an antimetabolite, a cytotoxic agent, a growth inhibitory agent, a taxane, a folate analog, or any combination thereof.

In some examples, the platinum-based chemotherapeutic agent comprises oxaliplatin, cisplatin, or carboplatin. In some examples, the platinum-based chemotherapeutic agent comprises oxaliplatin. In some examples, the oxaliplatin is administered to the subject in a dosing regimen comprising one or more dosing cycles. In some examples, the one or more dosing cycles comprise 21-day dosing cycles. In some examples, the oxaliplatin is administered to the subject on Day 1 of each 21-day dosing cycle. In some examples, the oxaliplatin is administered to the subject at a dose of 130 mg/m². In some examples, the oxaliplatin is administered to the subject intravenously.

In some examples, the antimetabolite comprises capecitabine, gemcitabine, 5-fluorouracil, or tegafur.

In some examples, the antimetabolite comprises capecitabine. In some examples, the capecitabine is administered to the subject in a dosing regimen comprising one or more dosing cycles. In some examples, the one or more dosing cycles comprise 21-day dosing cycles. In some examples, the capecitabine is administered to the subject on Days 1-14 of each 21-day dosing cycle. In some examples, the capecitabine is administered to the subject at a dose of 1000 mg/m² twice daily. In some examples, the capecitabine is administered to the subject orally.

In other examples, the antimetabolite comprises gemcitabine. In some examples, the gemcitabine is administered to the subject in a dosing regimen comprising one or more dosing cycles. In some examples, the one or more dosing cycles comprise 28-day dosing cycles. In some examples, the gemcitabine is administered to the subject on Days 1, 8, and 15 of each 28-day dosing cycle. In some examples, the gemcitabine is administered to the subject at a dose of 1000 mg/m² twice daily. In some examples, the gemcitabine is administered to the subject intravenously.

In some examples, the antimetabolite comprises tegafur.

In some examples, the antimetabolite comprises S-1 (tegafur-gimeracil-oteracil potassium). In some examples, the S-1 is administered to the subject in a dosing regimen comprising one or more dosing cycles. In some examples, the one or more dosing cycles comprise 21-day dosing cycles. In some examples, the S-1 is administered to the subject on Days 1-14 of each 21-day dosing cycle. In some examples, the S-1 is administered to the subject at a dose of 40 mg/m² twice daily. In some examples, the S-1 is administered to the subject orally.

In some examples, the taxane comprises nab-paclitaxel or paclitaxel.

In some examples, the taxane comprises nab-paclitaxel. In some examples, the nab-paclitaxel is administered to the subject in a dosing regimen comprising one or more dosing cycles. In some examples, the one or more dosing cycles comprise 28-day dosing cycles. In some examples, the nab-paclitaxel is administered to the subject on Days 1, 8, and 15 of each 28-day dosing cycle. In some examples, the nab-paclitaxel is administered to the subject at a dose of 125 mg/m². In some examples, the nab-paclitaxel is administered to the subject intravenously.

In some examples, the folate analog comprises leucovorin.

In another example, provided herein is a method of treating a subject having a locally advanced, recurrent, or metastatic NSCLC, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 1200 mg intravenously on Day 1 of each 21-day dosing cycle.

In another example, provided herein is an anti-latent TGF-beta 1 for use in treatment of a subject having a locally advanced, recurrent, or metastatic NSCLC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is the use of an anti-latent TGF-beta 1 in the manufacture of a medicament for treatment of a subject having a locally advanced, recurrent, or metastatic NSCLC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some examples, the NSCLC is histologically or cytologically confirmed metastatic nonsquamous NSCLC or metastatic squamous NSCLC.

In some examples, the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

In some examples, the subject has previously received a combination therapy comprising a platinum-containing chemotherapy regimen and a PD-1 axis binding antagonist.

In some examples, the subject has previously received a platinum-containing chemotherapy regimen and a PD-1 axis binding antagonist as individual regimens.

In some examples, the subject had disease progression or recurrence within 6 months of definitive therapy for locally advanced NSCLC.

In some examples, a tumor sample from the subject has been determined to have a detectable level of PD-L1 expression.

In another example, provided herein is a method of treating a subject having a locally advanced, recurrent, or metastatic gastric cancer, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) nivolumab at a dosage of 360 mg intravenously on Day 1 of each 21-day dosing cycle; (iii) (a) capecitabine at a dosage of 1000 mg/m² orally twice daily on Days 1-14 of each 21-day dosing cycle, or (b) S-1 at a dosage of 40 mg/m² orally twice daily on Days 1-14 of each 21-day dosing cycle; and (iv) oxaliplatin at a dosage of 130 mg/m² intravenously on Day 1 of each 21-day dosing cycle.

In another example, provided herein is an anti-latent TGF-beta 1 for use in treatment of a subject having a locally advanced, recurrent, or metastatic gastric cancer, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In another example, provided herein is the use of an anti-latent TGF-beta 1 in the manufacture of a medicament for treatment of a subject having a locally advanced, recurrent, or metastatic gastric cancer, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

In some examples, the subject has unresectable locally advanced or metastatic gastric cancer that is histologically confirmed to be adenocarcinoma.

In some examples, the gastric cancer comprises esophagogastric junction cancer.

In some examples, the gastric cancer is HER2-negative gastric cancer.

In some examples, the subject is previously untreated for gastric cancer and/or the subject has not been previously treated with a checkpoint inhibitor.

In another example, provided herein is a method of treating a subject having a locally advanced, recurrent, or metastatic PDAC, the method comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle.

In another example, provided herein is an anti-latent TGF-beta 1 for use in treatment of a subject having a locally advanced, recurrent, or metastatic PDAC, the treatment comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle.

In another example, provided herein is the use of an anti-latent TGF-beta 1 in the manufacture of a medicament for treatment of a subject having a locally advanced, recurrent, or metastatic PDAC, the treatment comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle.

In some examples, the subject has histologically or cytologically confirmed metastatic PDAC.

In some examples, the subject is previously untreated for the PDAC and/or the subject has not been previously treated with a checkpoint inhibitor.

In another example, provided herein is a method of treating a subject having a locally advanced, recurrent, or metastatic UC, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 1200 mg intravenously on Day 1 of each 21-day dosing cycle.

In another example, provided herein is an anti-latent TGF-beta 1 for use in treatment of a subject having a locally advanced, recurrent, or metastatic UC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 1200 mg intravenously on Day 1 of each 21-day dosing cycle.

In another example, provided herein is the use of an anti-latent TGF-beta 1 in the manufacture of a medicament for treatment of a subject having a locally advanced, recurrent, or metastatic UC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and (ii) atezolizumab at a dosage of 1200 mg intravenously on Day 1 of each 21-day dosing cycle.

In some examples, the subject has histologically documented, locally advanced (T4b, any N; or any T, N2-N3) UC, or metastatic UC (M1, Stage 4).

In some examples, the subject is previously untreated for UC.

In some examples, the subject is ineligible for cisplatin-containing chemotherapy.

In some examples, the subject is ineligible for cisplatin-containing chemotherapy as defined by any one of the following criteria: (i) impaired renal function in terms of glomerular filtration rate (GFR) of >30 mL/min but <60 mL/min as assessed by direct measurement or by calculation from serum or plasma creatinine; (ii) hearing loss of 25 dB at two contiguous frequencies as measured by audiometry; (iii) Grade 2 peripheral neuropathy; or (iv) ECOG Performance Status of 2.

In some examples, the subject has previously received at least one platinum-containing chemotherapy regimen.

In some examples, the subject had disease progression during or following treatment with at least one platinum-containing chemotherapy regimen.

In some examples, the at least one platinum-containing chemotherapy regimen comprised (i) gemcitabine and cisplatin or carboplatin or (ii) methotrexate, vinblastine, doxorubicin, and cisplatin.

In some examples, the subject received prior adjuvant or neoadjuvant chemotherapy and progressed within 12 months of treatment with a platinum-containing adjuvant or neoadjuvant regimen.

In some examples, the subject received one cycle of a platinum-containing chemotherapy regimen but discontinued because of a Grade 4 hematologic toxicity or a Grade 3-4 non-hematologic toxicity.

In some examples, the subject received no more than two prior regimens of treatment for the locally advanced or metastatic UC.

In some examples, the subject has not received prior treatment with a T-cell co-stimulating therapy or a checkpoint inhibitor.

In another example, provided herein is a method of treating a subject having a metastatic non-squamous NSCLC or a metastatic squamous NSCLC, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

In another example, provided herein is an anti-latent TGF-beta 1 for use in treatment of a subject having a metastatic non-squamous NSCLC or a metastatic squamous NSCLC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

In another example, provided herein is the use of an anti-latent TGF-beta 1 in the manufacture of a medicament for treatment of a subject having a metastatic non-squamous NSCLC or a metastatic squamous NSCLC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

In another example, provided herein is a method of treating a subject having a locally advanced, unresectable, or metastatic HER2-negative gastric cancer, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) nivolumab at a dosage of 360 mg intravenously on Day 1 of each 21-day dosing cycle; (iii) (a) capecitabine at a dosage of 1000 mg/m² orally twice daily on Days 1-14 of each 21-day dosing cycle, or (b) S-1 at a dosage of 40 mg/m² orally twice daily on Days 1-14 of each 21-day dosing cycle; and (iv) oxaliplatin at a dosage of 130 mg/m² intravenously on Day 1 of each 21-day dosing cycle, wherein the subject is previously untreated for the locally advanced, unresectable, or metastatic HER2-negative gastric cancer.

In another example, provided herein is an anti-latent TGF-beta 1 for use in treatment of a subject having a locally advanced, unresectable, or metastatic HER2-negative gastric cancer, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) nivolumab at a dosage of 360 mg intravenously on Day 1 of each 21-day dosing cycle; (iii) (a) capecitabine at a dosage of 1000 mg/m² orally twice daily on Days 1-14 of each 21-day dosing cycle, or (b) S-1 at a dosage of 40 mg/m² orally twice daily on Days 1-14 of each 21-day dosing cycle; and (iv) oxaliplatin at a dosage of 130 mg/m² intravenously on Day 1 of each 21-day dosing cycle, wherein the subject is previously untreated for the locally advanced, unresectable, or metastatic HER2-negative gastric cancer.

In another example, provided herein is the use of an anti-latent TGF-beta 1 in the manufacture of a medicament for treatment of a subject having a locally advanced, unresectable, or metastatic HER2-negative gastric cancer, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) nivolumab at a dosage of 360 mg intravenously on Day 1 of each 21-day dosing cycle; (iii) (a) capecitabine at a dosage of 1000 mg/m² orally twice daily on Days 1-14 of each 21-day dosing cycle, or (b) S-1 at a dosage of 40 mg/m² orally twice daily on Days 1-14 of each 21-day dosing cycle; and (iv) oxaliplatin at a dosage of 130 mg/m² intravenously on Day 1 of each 21-day dosing cycle, wherein the subject is previously untreated for the locally advanced, unresectable, or metastatic HER2-negative gastric cancer.

In another example, provided herein is a method of treating a subject having a metastatic PDAC, the method comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle, wherein the subject is previously untreated for the metastatic PDAC.

In another example, the invention provides an anti-latent TGF-beta 1 for use in treatment of a subject having a metastatic PDAC, the treatment comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle, wherein the subject is previously untreated for the metastatic PDAC.

In another example, provided herein is the use of an anti-latent TGF-beta 1 in the manufacture of a medicament for treatment of a subject having a metastatic PDAC, the treatment comprising administering to the subject an anti-cancer therapy in a dosing regimen comprising one or more 28-day dosing cycles, wherein the anti-cancer therapy comprises: (i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); (ii) atezolizumab at a dosage of 840 mg intravenously on Days 1 and 15 of each 28-day dosing cycle; (iii) nab-paclitaxel at a dose of 125 mg/m² on Days 1, 8, and 15 of each 28-day dosing cycle; and (iv) gemcitabine at a dosage of 1000 mg/m² intravenously on Days 1, 8, and 15 of each 28-day dosing cycle, wherein the subject is previously untreated for the metastatic PDAC.

In another example, provided herein is a method of treating a subject having a locally advanced or metastatic UC, the method comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein: the subject is previously untreated for the locally advanced or metastatic UC and is ineligible for cisplatin-containing chemotherapy; or the subject has previously received at least one platinum-containing chemotherapy regimen.

In another example, provided herein is an anti-latent TGF-beta 1 for use in treatment of a subject having a locally advanced or metastatic UC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein: the subject is previously untreated for the locally advanced or metastatic UC and is ineligible for cisplatin-containing chemotherapy; or the subject has previously received at least one platinum-containing chemotherapy regimen.

In another example, provided herein is the use of an anti-latent TGF-beta 1 in the manufacture of a medicament for treatment of a subject having a locally advanced or metastatic UC, the treatment comprising administering to the subject an anti-cancer therapy in a dosing regimen comprising one or more 21-day dosing cycles, wherein the anti-cancer therapy comprises: (i) an anti-latent TGF-beta 1 antibody at a dosage of 1800 mg intravenously on Day 1 of each 21-day dosing cycle, wherein the anti-latent TGF-beta 1 antibody comprises the following six HVRs: (a) an HVR-H1 comprising the amino acid sequence of SEAMN (SEQ ID NO: 1); (b) an HVR-H2 comprising the amino acid sequence of YIYTSGTTYRANWARG (SEQ ID NO: 2); (c) an HVR-H3 comprising the amino acid sequence of GTGIYDYYYWVMDL (SEQ ID NO:3); (d) an HVR-L1 comprising the amino acid sequence of QASQSISTYLA (SEQ ID NO: 4); (e) an HVR-L2 comprising the amino acid sequence of AASTLES (SEQ ID NO: 5); and (f) an HVR-L3 comprising the amino acid sequence of QSYSDGDSVG (SEQ ID NO: 6); and (ii) atezolizumab at a dosage of 1200 mg intravenously on Day 1 of each 21-day dosing cycle, wherein: the subject is previously untreated for the locally advanced or metastatic UC and is ineligible for cisplatin-containing chemotherapy; or the subject has previously received at least one platinum-containing chemotherapy regimen.

In any of the preceding examples, the subject may be administered the anti-latent TGF-beta 1 antibody until loss of clinical benefit or unacceptable toxicity.

In any of the preceding examples, each dosing cycle may have any suitable length, e.g., about 7 days, about 14 days, about 21 days, about 28 days, or longer. In some instances, each dosing cycle is about 21 days. In some instances, each dosing cycle is 21 days. In some instances, each dosing cycle is about 28 days. In some instances, each dosing cycle is 28 days.

In some examples, the method comprises between 1 and 40 dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 dosing cycles).

In some instances, a subject is administered a total of 1 to 50 doses of a anti-latent TGF-beta 1 antibody, e.g., 1 to 50 doses, 1 to 45 doses, 1 to 40 doses, 1 to 35 doses, 1 to 30 doses, 1 to 25 doses, 1 to 20 doses, 1 to 15 doses, 1 to 10 doses, 1 to 5 doses, 2 to 50 doses, 2 to 45 doses, 2 to 40 doses, 2 to 35 doses, 2 to 30 doses, 2 to 25 doses, 2 to 20 doses, 2 to 15 doses, 2 to 10 doses, 2 to 5 doses, 3 to 50 doses, 3 to 45 doses, 3 to 40 doses, 3 to 35 doses, 3 to 30 doses, 3 to 25 doses, 3 to 20 doses, 3 to 15 doses, 3 to 10 doses, 3 to 5 doses, 4 to 50 doses, 4 to 45 doses, 4 to 40 doses, 4 to 35 doses, 4 to 30 doses, 4 to 25 doses, 4 to 20 doses, 4 to 15 doses, 4 to 10 doses, 4 to 5 doses, 5 to 50 doses, 5 to 45 doses, 5 to 40 doses, 5 to 35 doses, 5 to 30 doses, 5 to 25 doses, 5 to 20 doses, 5 to 15 doses, 5 to 10 doses, 10 to 50 doses, 10 to 45 doses, 10 to 40 doses, 10 to 35 doses, 10 to 30 doses, 10 to 25 doses, 10 to 20 doses, 10 to 15 doses, 15 to 50 doses, 15 to 45 doses, 15 to 40 doses, 15 to 35 doses, 15 to 30 doses, 15 to 25 doses, 15 to 20 doses, 20 to 50 doses, 20 to 45 doses, 20 to 40 doses, 20 to 35 doses, 20 to 30 doses, 20 to 25 doses, 25 to 50 doses, 25 to 45 doses, 25 to 40 doses, 25 to 35 doses, 25 to 30 doses, 30 to 50 doses, 30 to 45 doses, 30 to 40 doses, 30 to 35 doses, 35 to 50 doses, 35 to 45 doses, 35 to 40 doses, 40 to 50 doses, 40 to 45 doses, or 45 to 50 doses. In particular instances, the doses may be administered intravenously.

The anti-latent TGF-beta 1 antibody and/or any additional therapeutic agent(s) (e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents) may be administered in any suitable manner known in the art. For example, the anti-latent TGF-beta 1 antibody and/or any additional therapeutic agent(s) may be administered sequentially (on different days) or concurrently (on the same day or during the same treatment cycle). In some instances, the anti-latent TGF-beta 1 antibody is administered prior to the additional therapeutic agent. In other instances, the anti-latent TGF-beta 1 antibody is administered after the additional therapeutic agent. In some instances, the anti-latent TGF-beta 1 antibody and/or any additional therapeutic agent(s) may be administered on the same day. In some instances, the anti-latent TGF-beta 1 antibody may be administered prior to an additional therapeutic agent that is administered on the same day. For example, the anti-latent TGF-beta 1 antibody may be administered prior to chemotherapy on the same day. In another example, the anti-latent TGF-beta 1 antibody may be administered prior to both chemotherapy and another drug (e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents) on the same day. In other instances, the anti-latent TGF-beta 1 antibody may be administered after an additional therapeutic agent that is administered on the same day. In yet other instances, the anti-latent TGF-beta 1 antibody is administered at the same time as the additional therapeutic agent. In some instances, the PD anti-latent TGF-beta 1 antibody is in a separate composition as the additional therapeutic agent. In some instances, the anti-latent TGF-beta 1 antibody is in the same composition as the additional therapeutic agent. In some instances, the anti-latent TGF-beta 1 antibody is administered through a separate intravenous line from any other therapeutic agent administered to the subject on the same day.

The anti-latent TGF-beta 1 antibody and any additional therapeutic agent(s) may be administered by the same route of administration or by different routes of administration. In some instances, the anti-latent TGF-beta 1 antibody is administered intravenously, intramuscularly, subcutaneously, topically, orally, transdermally, intraperitoneally, intraorbitally, by implantation, by inhalation, intrathecally, intraventricularly, or intranasally. In some instances, the additional therapeutic agent is administered intravenously, intramuscularly, subcutaneously, topically, orally, transdermally, intraperitoneally, intraorbitally, by implantation, by inhalation, intrathecally, intraventricularly, or intranasally.

In a preferred example, the anti-latent TGF-beta 1 antibody is administered intravenously.

Also provided herein are methods for treating cancer in a subject comprising administering to the subject an anti-cancer therapy comprising an effective amount of an anti-latent TGF-beta 1 antibody and/or a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents) in combination with another anti-cancer agent or cancer therapy. For example, an anti-latent TGF-beta 1 antibody may be administered in combination with an additional chemotherapy or chemotherapeutic agent (see definition above); a targeted therapy or targeted therapeutic agent; an immunotherapy or immunotherapeutic agent, for example, a monoclonal antibody; one or more cytotoxic agents (see definition above); or combinations thereof.

In some instances, the treatment may further comprise an additional therapy. Any suitable additional therapy known in the art or described herein may be used. The additional therapy may be radiation therapy, surgery, gene therapy, DNA therapy, viral therapy, RNA therapy, immunotherapy, bone marrow transplantation, nanotherapy, monoclonal antibody therapy, gamma irradiation, or a combination of the foregoing.

In some instances, the additional therapy is the administration of side-effect limiting agents (e.g., agents intended to lessen the occurrence and/or severity of side effects of treatment, such as anti-nausea agents, a corticosteroid (e.g., prednisone or an equivalent, e.g., at a dose of 1-2 mg/kg/day), hormone replacement medicine(s), and the like).

### III. Anti-Latent TGF-Beta 1 Antibodies

### A. Exemplary Anti-Latent TGF-Beta 1 Antibodies

Provided herein are anti-latent TGF-beta 1 antibodies that can be used in the methods and compositions described herein. Any suitable anti-latent TGF-beta 1 antibody may be used, including any anti-latent TGF-beta 1 antibody disclosed in WO 2021/039945 or herein.

In some examples, the anti-latent TGF-beta 1 antibody comprises at least one, two, three, four, five, or six HVRs selected from: (a) HVR-H1, HVR-H2, and HVR-H3 comprising the amino acid sequences of SEQ ID NO: 1, 2, and 3, respectively, and/or HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and/or HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and/or HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and/or HVR-L1, HVR-L2, and HVR-L3 comprising the amino acid sequences of SEQ ID NO: 28, 29, and 30, respectively. In particular examples, the anti-latent TGF-beta 1 antibody comprises at least one, two, three, four, five, or six HVRs selected from: (a) HVR-H1, HVR-H2, and HVR-H3 comprising the amino acid sequences of SEQ ID NO: 1, 2, and 3, respectively, and/or HVR-L1, HVR-L2, and HVR-L3 comprising the amino acid sequences of SEQ ID NO: 4, 5, and 6, respectively.

In some examples, the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively; (b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively; (c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or (d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively. In particular examples, the anti-latent TGF-beta 1 antibody comprises the following six HVRs: (a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively.

In some examples, the anti-latent TGF-beta 1 antibody comprises: (a) (i) a heavy chain variable domain (VH) sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 7, (ii) a light chain variable domain (VL) sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 8, or (iii) a VH sequence as defined in (i) and a VL sequence as defined in (ii); (b) (i) a VH sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 31, (ii) a VL sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 32, or (iii) a VH sequence as defined in (i) and a VL sequence as defined in (ii); (c) (i) a VH sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 33, (ii) a VL sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 34, (iii) a VH sequence as defined in (i) and a VL sequence as defined in (ii); or (d) (i) a VH sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 35, (ii) a VL sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 36, or (iii) a VH sequence as defined in (i) and a VL sequence as defined in (ii).

Any of the preceding anti-latent TGF-beta 1 antibodies above may include at least one, two, three, four, five, or six HVRs disclosed above.

In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-latent TGF-beta 1 antibody comprising that sequence retains the ability to bind to latent TGF-beta 1.

In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-latent TGF-beta 1 antibody comprising that sequence retains the ability to bind to latent TGF-beta 1.

In some examples, the anti-latent TGF-beta 1 antibody comprises: (a) a VH sequence of SEQ ID NO: 7 and a VL sequence of SEQ ID NO: 8; (b) a VH sequence of SEQ ID NO: 31 and a VL sequence of SEQ ID NO: 32; (c) a VH sequence of SEQ ID NO: 33 and a VL sequence of SEQ ID NO: 34; or (d) a VH sequence of SEQ ID NO: 35 and a VL sequence of SEQ ID NO: 36.

In particular examples, the anti-latent TGF-beta 1 antibody comprises a VH sequence of SEQ ID NO: 7 and a VL sequence of SEQ ID NO: 8.

In some examples, the anti-latent TGF-beta 1 antibody is a chimeric antibody.

In some examples, the anti-latent TGF-beta 1 antibody is a humanized antibody.

In some examples, the anti-latent TGF-beta 1 antibody is a full-length antibody.

In some examples, the anti-latent TGF-beta 1 antibody comprises: (a) a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 37 and a light chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 38; (b) a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 39 and a light chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 40; (c) a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 41 and a light chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 42; (d) a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 43 and a light chain comprising the amino acid sequence of SEQ ID NO: 44; (e) a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 45 and a light chain comprising the amino acid sequence of SEQ ID NO: 46; (f) a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 47 and a light chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 48; (g) a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 49 and a light chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 50; or (h) a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 51 and a light chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 52.

In some examples, the anti-latent TGF-beta 1 antibody comprises: (a) a heavy chain comprising the amino acid sequence of SEQ ID NO: 37 and a light chain comprising the amino acid sequence of SEQ ID NO: 38; (b) a heavy chain comprising the amino acid sequence of SEQ ID NO: 39 and a light chain comprising the amino acid sequence of SEQ ID NO: 40; (c) a heavy chain comprising the amino acid sequence of SEQ ID NO: 41 and a light chain comprising the amino acid sequence of SEQ ID NO: 42; (d) a heavy chain comprising the amino acid sequence of SEQ ID NO: 43 and a light chain comprising the amino acid sequence of SEQ ID NO: 44; (e) a heavy chain comprising the amino acid sequence of SEQ ID NO: 45 and a light chain comprising the amino acid sequence of SEQ ID NO: 46; (f) a heavy chain comprising the amino acid sequence of SEQ ID NO: 47 and a light chain comprising the amino acid sequence of SEQ ID NO: 48; (g) a heavy chain comprising the amino acid sequence of SEQ ID NO: 49 and a light chain comprising the amino acid sequence of SEQ ID NO: 50; or (h) a heavy chain comprising the amino acid sequence of SEQ ID NO: 51 and a light chain comprising the amino acid sequence of SEQ ID NO: 52.

In some examples, the anti-latent TGF-beta 1 antibody comprises a modified IgG1 Fc region having reduced effector function compared with a wild-type IgG1 Fc region.

In some examples, the modified IgG1 Fc region comprises a constant heavy (CH) region comprising one or more of the following substitutions: K214R, L235R, G236R, M428L, N434A, Q438R, and/or S440E (EU numbering). In some examples, the modified IgG1 Fc region comprises a CH region comprising the following substitutions: K214R, L235R, G236R, M428L, N434A, Q438R, and S440E (EU numbering).

In some examples, the CH region comprises the amino acid sequence of SEQ ID NO: 9 or an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 9.

In some examples, the CH region comprises the amino acid sequence of SEQ ID NO: 9.

In some examples, the modified IgG1 Fc region comprises a constant light (CL) domain comprising the amino acid sequence of SEQ ID NO: 10 or an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 10.

In some examples, the modified IgG1 Fc region comprises a CL domain comprising the amino acid sequence of SEQ ID NO: 10.

In some examples, the anti-latent TGF-beta 1 antibody comprises a heavy chain comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 11 and a light chain sequence comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or more) sequence identity to the amino acid sequence of SEQ ID NO: 12.

In particular examples, the anti-latent TGF-beta 1 antibody comprises a heavy chain comprising the amino acid sequence of SEQ ID NO: 11 and a light chain sequence comprising the amino acid sequence of SEQ ID NO: 12.

In some examples, the anti-latent TGF-beta 1 antibody is an antibody fragment that binds to latent TGF-beta 1.

Any of the anti-latent TGF-beta 1 antibodies disclosed herein may include one or more post-translational modifications. Post-translational modifications include but are not limited to a modification of glutamine or glutamate in N-terminus of heavy chain or light chain to pyroglutamic acid by pyroglutamylation.

In some examples, the anti-latent TGF-beta 1 antibody is an antibody that competes for binding to latent TGF-beta 1 and/or that binds to the same epitope as any of the anti-latent TGF-beta 1 antibodies disclosed herein.

In a further aspect, an anti-latent TGF-beta 1 antibody as disclosed herein or according to any of the above examples may incorporate any of the features, singly or in combination, as described in Sections 1-7 below:

### 1. Antibody Binding Activity

In certain embodiments, an antibody provided herein has a dissociation constant (KD) of 1 micromolar or less, 100 nM or less, 10 nM or less, 1 nM or less, 0.1 nM or less, 0.01 nM or less, or 0.001 nM or less (e.g., 10⁻⁸ M or less, e.g., from 10⁻⁸ M to 10⁻¹³ M, e.g., from 10⁻⁹ M to 10⁻¹³ M).

In one embodiment, binding activity of an antibody is measured by a radiolabeled antigen binding assay (RIA) and represented by KD. In one embodiment, an RIA is performed with the Fab version of an antibody of interest and its antigen. For example, solution binding activity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 microgram/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees Celsius (C)). In a non-adsorbent plate (Nunc #269620), 100 pM or 26 pM (¹²⁵I)-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 microliter/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

In one embodiment, for measuring binding activity of an antibody, ligand-capturing methods, for example, using BIACORE® T200 or BIACORE®4000 (GE Healthcare, Uppsala, Sweden), which rely upon surface plasmon resonance analysis methods as the measurement principle, are used. BIACORE® Control Software is used for operation of devices. In one embodiment, amine-coupling kit (GE Healthcare, Uppsala, Sweden) is used according to the manufacturer's instructions to let a molecule for ligand capturing, for example, an anti-tag antibody, an anti-IgG antibody, protein A, etc. fixed onto a sensor chip (GE Healthcare, Uppsala, Sweden) coated with carboxymethyldextran. The ligand-capturing molecule is diluted with a 10 mM sodium acetate solution at an appropriate pH and is injected at an appropriate flow rate and for an appropriate injection time. Binding activity measurements are measured using a 0.05% polysorbate 20 (Tween®-20)-containing buffer as a measurement buffer, at a flow rate of 10⁻³⁰ microliter/minute, and at a measurement temperature of preferably at 25 degrees C. or 37 degrees C. For the measurement carried out with an antibody captured by the ligand-capturing molecule as a ligand, an antibody is injected to let a target amount of the antibody captured, and then a serial dilution of an antigen and/or an Fc receptor (analyte) prepared using the measurement buffer is injected. For the measurement carried out with an antigen and/or an Fc receptor captured by the ligand-capturing molecule as a ligand, an antigen and/or an Fc receptor is injected to let a target amount thereof captured, and then a serial dilution of an antibody (analyte) prepared using the measurement buffer is injected.

In one embodiment, the measurement results are analyzed using BIACORE® Evaluation Software. Kinetics parameter calculation is carried out by fitting sensorgrams of association and dissociation at the same time using a 1:1 binding model, and an association rate (kₒₙ or kₐ), a dissociation rate (k_{off} or k_{d}), and an equilibrium dissociation constant (KD) may be calculated. For the case of weak binding activity, in particular, for the cases where dissociation is fast and kinetics parameters are difficult to calculate, the Steady state model may be used to calculate the equilibrium dissociation constant (KD). As additional parameters concerning binding activity, “binding amount of analyte per unit ligand amount” may be calculated by dividing a binding amount of analyte (resonance unit: RU) at a specific concentration by an amount of captured ligand.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

The present invention also relates to antigen-binding molecules which bind to TGF-beta 1, which include, but are not limited to, for example, minibodies (low molecular weight antibodies), and scaffold proteins. In the present disclosure, any scaffold protein is acceptable as long as it is a peptide that has a stable three-dimensional structure and is capable of binding to at least an antigen. Such peptides include, for example, fragments of antibody variable regions, fibronectin, protein A domain, LDL receptor A domain, lipocalin, and other molecules described in Nygren et al. (Current Opinion in Structural Biology, (1997) 7:463-469; Journal of Immunol Methods, (2004) 290:3-28), Binz et al. (Nature Biotech. (2005) 23:1257-1266), and Hosse et al. (Protein Science, (2006) 15:14-27). When referring to such an antibody, e.g., “anti-latent TGF-beta 1 antibody” should be replaced with “anti-latent TGF-beta 1 antigen-binding molecule” in the context of the present specification.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and binding activity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or binding activity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-binding activity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g., a bispecific antibody. Multispecific antibodies are monoclonal antibodies that have binding specificities for at least two different sites. In certain embodiments, one of the binding specificities is for TGF-beta 1 and the other is for any other antigen. In certain embodiments, bispecific antibodies may bind to two different epitopes of TGF-beta 1. Bispecific antibodies may also be used to localize cytotoxic agents to cells which express TGF-beta 1. Bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting Fab” or “DAF” comprising an antigen binding site that binds to TGF-beta 1 as well as another, different antigen (see, US 2008/0069820, for example).

### 7. Antibody Variants

In certain embodiments, amino acid sequence variants of the antibodies provided herein are contemplated. For example, it may be desirable to improve the binding activity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, lie;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased binding activity, reduced immunogenicity) relative to the parent antibody and/or will have substantially retained certain biological properties of the parent antibody. An exemplary substitutional variant is a binding activity matured antibody, which may be conveniently generated, e.g., using phage display-based binding activity maturation techniques such as those described herein. Briefly, one or more HVR residues are mutated and the variant antibodies displayed on phage and screened for a particular biological activity (e.g. binding activity).

Alterations (e.g., substitutions) may be made in HVRs, e.g., to improve antibody binding activity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding activity. Binding activity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of binding activity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired binding activity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding activity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be analyzed to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion of an enzyme (e.g., for ADEPT) or a polypeptide which increases the plasma half-life of the antibody to the N- or C-terminus of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions. In another embodiment, a human Fc variant may comprise a chimeric human Fc region sequence (e.g., a human IgG1/4 or human IgG2/4 Fc region), or a chimeric human Fc region sequence which further comprises an amino acid modification (e.g., a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT1™ non-radioactive cytotoxicity assay for flow cytometry (CellTechnology, Inc. Mountain View, CA; and CytoTox® 96 non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an anti-latent TGF-beta 1 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp2/0 cell). In one embodiment, a method of making an anti-latent TGF-beta 1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-latent TGF-beta 1 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### IV. Assessment of PD-L1 Expression

The expression of PD-L1 may be assessed in a subject treated according to any of the methods and compositions for use described herein or known in the art. The methods and compositions for use may include determining the expression level of PD-L1 in a biological sample (e.g., a tumor sample) obtained from the subject. In other examples, the expression level of PD-L1 in a biological sample (e.g., a tumor sample) obtained from the subject has been determined prior to initiation of treatment or after initiation of treatment. PD-L1 expression may be determined using any suitable approach. For example, PD-L1 expression may be determined as described in U.S. patent application Ser. No. 15/787,988 or U.S. Pat. No. 11,535,671. Any suitable tumor sample may be used, e.g., a formalin-fixed and paraffin-embedded (FFPE) tumor sample, an archival tumor sample, a fresh tumor sample, or a frozen tumor sample.

For example, PD-L1 expression may be determined in terms of the percentage of a tumor sample comprised by tumor-infiltrating immune cells expressing a detectable expression level of PD-L1, as the percentage of tumor-infiltrating immune cells in a tumor sample expressing a detectable expression level of PD-L1, and/or as the percentage of tumor cells in a tumor sample expressing a detectable expression level of PD-L1. It is to be understood that in any of the preceding examples, the percentage of the tumor sample comprised by tumor-infiltrating immune cells may be in terms of the percentage of tumor area covered by tumor-infiltrating immune cells in a section of the tumor sample obtained from the subject, for example, as assessed by IHC using an anti-PD-L1 antibody (e.g., the SP142 antibody). Any suitable anti-PD-L1 antibody may be used, including, e.g., SP142 (Ventana), SP263 (Ventana), 22C3 (Dako), 28-8 (Dako), E1 L3N (Cell Signaling Technology), 4059 (ProSci, Inc.), h5H1 (Advanced Cell Diagnostics), and 9A11. In some examples, the anti-PD-L1 antibody is SP142. In other examples, the anti-PD-L1 antibody is SP263.

In some examples, a tumor sample obtained from the subject has a detectable expression level of PD-L1 in less than 1% of the tumor cells in the tumor sample, in 1% or more of the tumor cells in the tumor sample, in from 1% to less than 5% of the tumor cells in the tumor sample, in 5% or more of the tumor cells in the tumor sample, in from 5% to less than 50% of the tumor cells in the tumor sample, or in 50% or more of the tumor cells in the tumor sample.

In some examples, a tumor sample obtained from the subject has a detectable expression level of PD-L1 in tumor-infiltrating immune cells that comprise less than 1% of the tumor sample, more than 1% of the tumor sample, from 1% to less than 5% of the tumor sample, more than 5% of the tumor sample, from 5% to less than 10% of the tumor sample, or more than 10% of the tumor sample.

In some examples, tumor samples may be scored for PD-L1 positivity in tumor-infiltrating immune cells and/or in tumor cells according to the criteria for diagnostic assessment shown in Table B and/or Table C, respectively.

TABLE B
Tumor-infiltrating immune cell (IC) IHC diagnostic criteria

| PD-L1 Diagnostic Assessment | IC Score |
|---|---|
| Absence of any discernible PD-L1 staining | IC0 |
| OR | |
| Presence of discernible PD-L1 staining of any | |
| intensity in tumor-infiltrating immune cells | |
| covering <1% of tumor area occupied by tumor cells, | |
| associated intratumoral stroma, and contiguous | |
| peri-tumoral desmoplastic stroma | |
| Presence of discernible PD-L1 staining of any | IC1 |
| intensity in tumor-infiltrating immune cells | |
| covering ≥1% to <5% of tumor area occupied by tumor cells, | |
| associated intratumoral stroma, and contiguous | |
| peri-tumoral desmoplastic stroma | |
| Presence of discernible PD-L1 staining of any | IC2 |
| intensity in tumor-infiltrating immune cells | |
| covering ≥5% to <10% of tumor area occupied by tumor | |
| cells, associated intratumoral stroma, and | |
| contiguous peri-tumoral desmoplastic stroma | |
| Presence of discernible PD-L1 staining of any | IC3 |
| intensity in tumor-infiltrating immune cells | |
| covering ≥10% of tumor area occupied by tumor cells, | |
| associated intratumoral stroma, and contiguous | |
| peri-tumoral desmoplastic stroma | |

TABLE C
Tumor cell (TC) IHC diagnostic criteria

| PD-L1 Diagnostic Assessment | TC Score |
|---|---|
| Absence of any discernible PD-L1 staining | TC0 |
| OR | |
| Presence of discernible PD-L1 staining of any | |
| intensity in <1% of tumor cells | |
| Presence of discernible PD-L1 staining of any | TC1 |
| intensity in ≥1% to <5% of tumor cells | |
| Presence of discernible PD-L1 staining of any | TC2 |
| intensity in ≥5% to <50% of tumor cells | |
| Presence of discernible PD-L1 staining of any | TC3 |
| intensity in ≥50% of tumor cells | |

### V. PD-1 Axis Binding Antagonists

In some examples, an anti-latent TGF-beta 1 antibody is administered in combination with a PD-1 axis binding antagonist. PD-1 axis binding antagonists may include PD-L1 binding antagonists, PD-1 binding antagonists, and PD-L2 binding antagonists. Any suitable PD-1 axis binding antagonist may be used.

### A. PD-L1 Binding Antagonists

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody is atezolizumab.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In a still further specific aspect, the anti-PD-L1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In still a further instance, the effector-less Fc mutation is an N297A substitution in the constant region. In some instances, the isolated anti-PD-L1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

In a further aspect, an anti-PD-L1 antibody may incorporate any of the features, singly or in combination, as described in Sections 1-7 of Section III(A) above.

### B. PD-1 Binding Antagonists

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances, the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-110A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In a still further specific aspect, the anti-PD-1 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-1 antibody is aglycosylated.

In a further aspect, an anti-PD-1 antibody may incorporate any of the features, singly or in combination, as described in Sections 1-7 of Section III(A) above.

### C. PD-L2 Binding Antagonists

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

In a further aspect, an anti-PD-L2 antibody may incorporate any of the features, singly or in combination, as described in Sections 1-7 of Section III(A) above.

### VI. Chemotherapeutic Agents and Chemotherapy

Provided herein are methods for treating cancer in a subject comprising administering to the subject an anti-cancer therapy comprising an anti-latent TGF-beta 1 antibody and one or more chemotherapeutic agents. Also provided are related compositions (e.g., pharmaceutical compositions) for use, kits, and articles of manufacture. Any of the methods, compositions for use, kits, or articles of manufacture described herein may include or involve any of the agents described herein.

Any suitable chemotherapeutic agents may be used, including any chemotherapeutic agents or combinations of chemotherapeutic agents disclosed herein. In some examples, the one or more chemotherapeutic agents comprise a platinum-based chemotherapeutic agent, an antimetabolite, a cytotoxic agent, a growth inhibitory agent, a taxane, a folate analog, or any combination thereof.

In some examples, the chemotherapeutic agent or combination of chemotherapeutic agents may be a standard of care (SOC) therapy for a cancer.

Any suitable platinum-based chemotherapeutic agent may be used. Exemplary platinum-based chemotherapeutic agents include, but are not limited to, cisplatin, carboplatin, oxaliplatin, nedaplatin, triplatin tetranitrate, phenanthriplatin, picoplatin, lipoplatin, and satraplatin. In some examples, the platinum-based chemotherapeutic agent comprises oxaliplatin, cisplatin, or carboplatin. In some examples, the platinum-based chemotherapeutic agent comprises oxaliplatin. In some examples, the oxaliplatin is administered to the subject in a dosing regimen comprising one or more dosing cycles. In some examples, the one or more dosing cycles comprise 21-day dosing cycles. In some examples, the oxaliplatin is administered to the subject on Day 1 of each 21-day dosing cycle. In some examples, the oxaliplatin is administered to the subject at a dose of 130 mg/m². In some examples, the oxaliplatin is administered to the subject intravenously.

Any suitable antimetabolite may be used. In some examples, the antimetabolite comprises capecitabine, gemcitabine, 5-fluorouracil, or tegafur.

In some examples, the antimetabolite comprises capecitabine. In some examples, the capecitabine is administered to the subject in a dosing regimen comprising one or more dosing cycles. In some examples, the one or more dosing cycles comprise 21-day dosing cycles. In some examples, the capecitabine is administered to the subject on Days 1-14 of each 21-day dosing cycle. In some examples, the capecitabine is administered to the subject at a dose of 1000 mg/m² twice daily. In some examples, the capecitabine is administered to the subject orally.

In other examples, the antimetabolite comprises gemcitabine. In some examples, the gemcitabine is administered to the subject in a dosing regimen comprising one or more dosing cycles. In some examples, the one or more dosing cycles comprise 28-day dosing cycles. In some examples, the gemcitabine is administered to the subject on Days 1, 8, and 15 of each 28-day dosing cycle. In some examples, the gemcitabine is administered to the subject at a dose of 1000 mg/m² twice daily. In some examples, the gemcitabine is administered to the subject intravenously.

In some examples, the antimetabolite comprises tegafur.

In some examples, the antimetabolite comprises S-1 (tegafur-gimeracil-oteracil potassium). In some examples, the S-1 is administered to the subject in a dosing regimen comprising one or more dosing cycles. In some examples, the one or more dosing cycles comprise 21-day dosing cycles. In some examples, the S-1 is administered to the subject on Days 1-14 of each 21-day dosing cycle. In some examples, the S-1 is administered to the subject at a dose of 40 mg/m² twice daily. In some examples, the S-1 is administered to the subject orally.

Any suitable taxane may be used. Exemplary taaxanes include, but are not limited to, paclitaxel (i.e., TAXOL®, CAS #33069-62-4), docetaxel (i.e., TAXOTERE®, CAS #114977-28-5), larotaxel, cabazitaxel, milataxel, tesetaxel, and/or orataxel. In some examples, the taxane comprises nab-paclitaxel or paclitaxel.

In some examples, the taxane comprises nab-paclitaxel. In some examples, the nab-paclitaxel is administered to the subject in a dosing regimen comprising one or more dosing cycles. In some examples, the one or more dosing cycles comprise 28-day dosing cycles. In some examples, the nab-paclitaxel is administered to the subject on Days 1, 8, and 15 of each 28-day dosing cycle. In some examples, the nab-paclitaxel is administered to the subject at a dose of 125 mg/m². In some examples, the nab-paclitaxel is administered to the subject intravenously.

Any suitable folate analog may be used. Exemplary, non-limiting folate analogs include leucovorin and levoleucovorin. In some examples, the folate analog comprises leucovorin.

### VII. Pharmaceutical Compositions and Formulations

Also provided herein are pharmaceutical compositions and formulations comprising an anti-latent TGF-beta 1 antibody and, optionally, a pharmaceutically acceptable carrier. The disclosure also provides pharmaceutical compositions and formulations comprising one or more additional therapeutic agents, e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents, and optionally, a pharmaceutically acceptable carrier.

Pharmaceutical compositions and formulations as described herein can be prepared by mixing the active ingredients (e.g., a PD-1 axis binding antagonist) having the desired degree of purity with one or more optional pharmaceutically acceptable carriers (see, e.g., Remington's Pharmaceutical Sciences 16th edition, Osol, A. Ed. (1980)), e.g., in the form of lyophilized formulations or aqueous solutions.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

### VIII. Articles of Manufacture or Kits

In another aspect, provided herein is an article of manufacture or a kit comprising an anti-latent TGF-beta 1 antibody. In some instances, the article of manufacture or kit further comprises package insert comprising instructions for using the anti-latent TGF-beta 1 antibody to treat or delay progression of cancer in a subject. In some instances, the article of manufacture or kit further comprises package insert comprising instructions for using an anti-latent TGF-beta 1 antibody in combination with one or more additional therapeutic agents (e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents) to treat or delay progression of cancer in a subject. Any of the anti-latent TGF-beta 1 antibodies and additional therapeutic agents (e.g., checkpoint inhibitors such as PD-1 axis binding antagonists and/or one or more chemotherapeutic agents) described herein may be included in the article of manufacture or kits.

In some instances, the anti-latent TGF-beta 1 antibody and the one or more additional therapeutic agents (e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some instances, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some instances, the article of manufacture further includes one or more of another agent (e.g., an additional chemotherapeutic agent or anti-neoplastic agent). Suitable containers for the one or more agents include, for example, bottles, vials, bags and syringes.

Any of the articles of manufacture or kits may include instructions to administer an anti-latent TGF-beta 1 antibody and/or one or more additional therapeutic agents (e.g., a checkpoint inhibitor (e.g., a PD-1 axis antagonist (e.g., an anti-PD-L1 antibody (e.g., atezolizumab) or an anti-PD-1 antibody (e.g., nivolumab))) and/or one or more chemotherapeutic agents) to a subject in accordance with any of the methods described herein, e.g., any of the methods set forth in Section II above.

### EXAMPLES

### Example 1. A Phase Ib, Open-Label, Multicenter Dose-Expansion Study Evaluating the Safety, Pharmacokinetics, and Activity of SOF10 in Combination with a Checkpoint Inhibitor with or without Standard-of-Care Chemotherapy in Patients with Locally Advanced or Metastatic Solid Tumors

A Phase Ib, open-label, multicenter, dose-expansion study was designed to evaluate the safety, tolerability, pharmacokinetics, immunogenicity, and preliminary anti-tumor activity of the anti-latent TGF-beta 1 antibody SOF10 when administered in combination with a checkpoint inhibitor (CPI), e.g., an anti-PD-L1 antibody such as atezolizumab or an anti-PD-1 antibody such as nivolumab, with or without standard of care (SOC) chemotherapy in patients with locally advanced or metastatic solid tumors.

In this example, “study treatment” refers to the combination of all treatments assigned to patients as part of this study (e.g., SOF10 in combination with atezolizumab or nivolumab, with or without SOC chemotherapy).

### A. Objectives and Endpoints

This study evaluates the safety, tolerability, pharmacokinetics, immunogenicity, and preliminary anti-tumor activity of SOF10 when administered in combination with a checkpoint inhibitor (i.e., atezolizumab or nivolumab) with or without standard-of-care chemotherapy in patients with non-small cell lung cancer, gastric cancer, and pancreatic ductal adenocarcinoma. A substudy for urothelial carcinoma (UC) is described in Example 2. Specific objectives and corresponding endpoints for the study are outlined below in Table 1.

TABLE 1
Objectives and Corresponding Endpoints

| | Corresponding Endpoints |
|---|---|
| Primary Objective | |
| To evaluate the safety and tolerability of | Incidence and severity of adverse events |
| SOF10 when administered in combination | (AE), with severity determined according to |
| with a CPI with or without SOC | the National Cancer Institute's Common |
| chemotherapy. | Terminology Criteria for AE (CTCAE) v5.0 |
| | grading scale. |
| | Change from baseline in targeted vital signs. |
| | Change from baseline in targeted clinical |
| | laboratory test results. |
| Secondary Objectives | |
| To characterize the pharmacokinetics of | Plasma concentration of SOF10 at specified |
| SOF10. | timepoints and relevant pharmacokinetic (PK) |
| | parameters. |
| To evaluate the immune response to SOF10. | Prevalence of anti-drug antibodies (ADAs) to |
| | SOF10 at baseline and incidence of ADAs to |
| | SOF10 during the study. |
| To make a preliminary assessment of the | Confirmed (objective response rate) ORR, |
| activity of SOF10 when administered in | defined as the proportion of patients with a |
| combination with a CPI with or without SOC | complete response (CR) or partial response |
| chemotherapy. | (PR) on two consecutive occasions ≥4 weeks |
| | apart, as determined by the investigator |
| | according to Response Evaluation Criteria in |
| | Solid Tumors, Version 1.1 (RECIST v1.1). |
| | Duration of response DOR, defined as the |
| | time from the first occurrence of a |
| | documented confirmed objective response to |
| | disease progression or death from any cause |
| | during the study (whichever occurs first), as |
| | determined by the investigator according to |
| | RECIST v1.1. |
| | PFS, defined as the time from the first SOF10 |
| | treatment day to the first occurrence of |
| | disease progression or death from any cause |
| | during the study (whichever occurs first), as |
| | determined by the investigator according to |
| | RECIST v1.1 |
| To identify a recommended dose and regimen | Relationship between SOF10 dose and |
| for SOF10 for subsequent studies. | safety, PK, activity, and immunogenicity |
| | endpoints. |
| Exploratory Objectives | |
| To make a preliminary assessment of the | Overall survival (OS), defined as the time |
| activity of SOF10 when administered in | from the first SOF10 treatment day to death |
| combination with a CPI with or without SOC | from any cause. |
| chemotherapy. | Change from baseline in carbohydrate |
| | antigen 19-9 (CA19-9) at subsequent |
| | timepoints (pancreatic ductal adenocarcinoma |
| | (PDAC) cohort only). |
| To characterize the pharmacokinetics of | Serum concentration of atezolizumab at |
| atezolizumab | specified timepoints and relevant PK |
| | parameters (non-small cell lung cancer |
| | (NSCLC) and PDAC cohorts only) |
| To evaluate the immune response to | Prevalence of ADAs to atezolizumab at |
| atezolizumab | baseline and incidence of ADAs to |
| | atezolizumab during the study (NSCLC and |
| | PDAC cohorts only) |
| To evaluate potential relationships between | Relationship between plasma concentration |
| drug exposure and the efficacy and safety of | or PK parameters for SOF10 and safety |
| SOF10. | endpoints. |
| | Relationship between plasma concentration |
| | or PK parameters for SOF10 and activity |
| | endpoints. |
| To evaluate potential effects of ADAs. | Relationship between ADA status and safety, |
| | PK, or activity endpoints. |

ADA = anti-drug antibody; CA19-9 = carbohydrate antigen 19-9; CPI = checkpoint inhibitor; CR = complete response; CTCAE v5.0 = National Cancer Institute's Common Terminology Criteria for Adverse Events, Version 5.0; DOR = duration of response; NSCLC = non-small cell lung cancer; ORR = objective response rate; OS = overall survival; PDAC = pancreatic ductal adenocarcinoma; PFS = progression-free survival; PK = pharmacokinetic; PR = partial response; RECIST v1.1 = Response Evaluation Criteria in Solid Tumors, Version 1.1; SOC = standard of care.

Definitions for the criteria used to determine objective tumor response for target lesions are as follows:
Complete response (CR): disappearance of all target lesions
Any pathological lymph nodes (whether target or non-target) must have reduction in short axis to <10 mm.
Partial response (PR): at least a 30% decrease in the sum of diameters of target lesions, taking as reference the baseline sum of diameters
Progressive disease (PD): at least a 20% increase in the sum of diameters of target lesions, taking as reference the smallest sum on study (nadir), including baseline. In addition to the relative increase of 20%, the sum must also demonstrate an absolute increase of at least 5 mm.

The appearance of one or more new lesions is also considered progression.
Stable disease (SD): neither sufficient shrinkage to qualify for PR nor sufficient increase to qualify for PD, taking as reference the smallest sum on study.

### B. Study Design

i. Description of the Study

This is a Phase Ib, open-label, multicenter, dose-expansion study designed to evaluate the safety, tolerability, pharmacokinetics, immunogenicity, and preliminary anti-tumor activity of SOF10 when administered in combination with a CPI (e.g., atezolizumab or nivolumab) with or without SOC chemotherapy in patients with locally advanced or metastatic solid tumors. Several key aspects of the study design and study population are summarized below in Table 2.

TABLE 2
Summary of Study Design and Study Population

| | | | |
|---|---|---|---|
| Phase: | Phase Ib | Population Type: | Adult patients |
| Control | None | Population | Locally advanced or metastatic solid |
| Method: | | Diagnosis or | tumors (non-small cell lung cancer, |
| | | Condition: | gastric cancer, pancreatic ductal |
| | | | adenocarcinoma) |
| Interventional | Single group | Population Age: | ≥18 years |
| Model: | | | |
| Test | SOF10, atezolizumab, | Site Distribution: | Multi-site and multi-region |
| Compounds: | nivolumab, gemcitabine, | | |
| | nab-paclitaxel, capecitabine, | | |
| | oxaliplatin, S-1 | | |
| Active | Not applicable | Study | Not applicable |
| Comparator: | | Intervention | |
| | | Assignment | |
| | | Method: | |
| Number of | 1 | Number of | Approximately 120 |
| Arms: | | Participants to | |
| | | Be Enrolled: | |

The study includes a screening period of up to 28 days, a treatment period, a minimum follow-up period of 90 days after treatment, and survival follow-up.

Approximately 120 patients are to be enrolled in this study in three non-randomized indication-specific cohorts, at approximately 30 global investigative sites. The patient populations include patients with NSCLC (Cohort A), GC (Cohort B), and PDAC (Cohort C); a substudy for UC is described in Example 2. Each cohort enrolls up to approximately 40 patients and consist of two stages: an initial safety run-in stage and an expansion stage. The safety run-in stage enrolls a total of approximately 18 patients (e.g., 6 patients for each treatment regimen). Patients in the safety run-in receive at least one cycle of study treatment, with both SOF10 and atezolizumab or nivolumab given concurrently starting from Cycle 1. Accrual in the expansion stage for a given cohort begins after the safety run-in stage for that safety run-in group has been fully enrolled. Efficacy analyses is performed after approximately 15 patients have completed at least one tumor assessment in a given cohort.

Patient populations include patients with NSCLC (Cohort A), gastric cancer (GC) (Cohort B), and PDAC (Cohort C). Each cohort includes two stages: an initial safety run-in stage and an expansion stage. Patients in the safety run-in receive at least one cycle of study treatment, with both SOF10 and atezolizumab or nivolumab given concurrently starting from Cycle 1. Efficacy analyses are performed based on a tumor assessment.

Patients in this study are initially assessed for eligibility during the screening period (lasting s 28 days). Following confirmation of eligibility, patients receive SOF10 in combination with a CPI (Cohort A); or SOF10 in combination with a CPI and SOC chemotherapy (Cohorts B and C) (e.g., see Table 4). Patients receive study treatment in 21-day cycles (Cohorts A and B) or 28-day cycles (Cohort C). Treatment is continued as long as patients are experiencing clinical benefit in the opinion of the investigator (e.g., in the absence of unacceptable toxicity or symptomatic deterioration attributed to disease progression). Patients are permitted to continue study treatment after disease progression per Response Evaluation Criteria in Solid Tumors, Version 1.1 (RECIST v1.1) if they meet certain criteria, including, but not limited to, evidence of clinical benefit as assessed by the investigator (e.g., clinician).

Patients undergo tumor assessments at screening (baseline) and at regular intervals during the study, which is measured by RECIST v1.1. All patients are closely monitored for adverse events throughout the study and for at least 90 days after the final dose of study treatment or until initiation of another systemic anti-cancer therapy, whichever occurs first. Adverse events are graded according to the National Cancer Institute's Common Terminology Criteria for Adverse Events, Version 5.0 (CTCAE v5.0).

All patients in the study are followed for survival and subsequent anti-cancer therapy information approximately every 3 months until death, loss to follow-up, or Sponsor decision to discontinue survival follow-up because no further clinical development of SOF10 is planned or the study is terminated by the Sponsor, unless the patient requests to be withdrawn from follow-up.

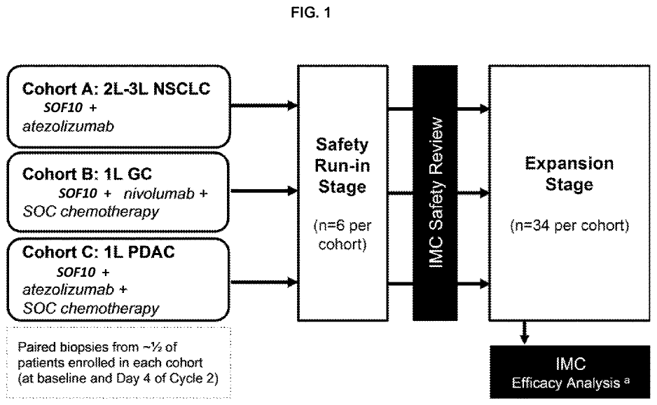

The study design is shown in FIG. 1. Study treatments are shown in, e.g., Table 4.

### Duration of Participation

The study includes a screening period of up to 28 days, a treatment period, a minimum follow-up period of 90 days after treatment, and survival follow-up. Treatment is continued as long as patients are experiencing clinical benefit in the opinion of the investigator. The total duration of study participation for each patient is expected to range from 1 day to more than 12 months.

### Safety Run-In Stage

Approximately 18 patients are to be enrolled in a safety run-in stage: approximately 6 patients from each cohort.

Dosing for each cohort in the safety run-in stage are performed as described in Table 4. After 6 patients in a safety run-in group (e.g., Cohort A, Cohort B, or Cohort C) have completed at least one cycle of study treatment in the safety run-in stage, safety data are formally reviewed. Continued enrollment or recommended changes to the conduct of the study are determined based on the investigator's (e.g., a clinician) review of the safety data.

### Expansion Stage

Accrual in the expansion stage for a given cohort begins after the safety run-in stage for that safety run-in group has been fully enrolled (e.g., 6 patients are enrolled in Cohorts A, B, or C). Additional patients may be enrolled in the expansion stage of this Phase Ib study. Patients receive study treatment as shown in Table 4 to obtain additional safety, tolerability, and PK data, as well as preliminary evidence of clinical activity.

### Serial Biopsies

Up to approximately half of the patients enrolled in each cohort must have safely accessible tumor lesions and consent to pretreatment and on-treatment biopsies (core needle, punch, forceps, or excisional/incisional). Safely accessible lesions are defined as those lesions that would not place the patient at undue risk from the procedure per the investigator's (e.g., a clinician) discretion (e.g., risk of hemorrhage due to proximity to major blood vessels, etc.).

Patients with archival tumor specimens (e.g., collected within 3 months of screening) may decline to undergo a pretreatment baseline biopsy and submit the archival specimen instead. The on-treatment biopsy must still be performed, preferably from the same lesion or organ as the archival tumor specimen, if feasible.

Serial biopsy patients whose pretreatment biopsy is found to be non-evaluable (e.g., due to insufficient material or lack of tumor cells in the sample) may still receive study treatment. If the pretreatment biopsy is found to be non-evaluable prior to Cycle 2, Day 4, the patient may decline to undergo an on-treatment biopsy. Such patients, as well as patients whose on-treatment biopsy is found to be non-evaluable or any patients with biopsies without sufficient viable tumor content, may be replaced for the purpose of serial biopsy assessment of evaluable patients in each cohort. Patients who provide pretreatment biopsies should also submit available archival tumor specimens. If no archival tumor specimen is available, the pretreatment biopsy is sufficient to meet tissue eligibility requirements for the study.

Treatment after Disease Progression

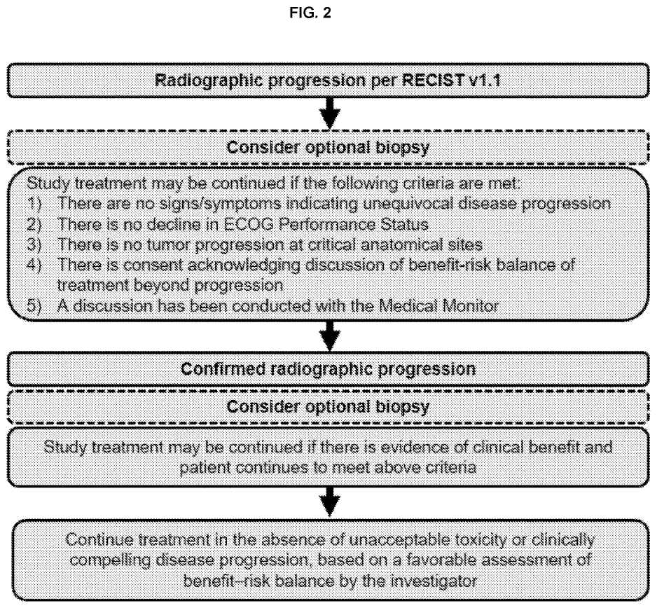

Patients may continue study treatment in the safety run-in or expansion stages of the study after standard criteria per RECIST v1.1 for progressive disease are met at the investigator's discretion, provided that the patients meet all of the following criteria (e.g., see FIG. 2):
Absence of symptoms and signs (including worsening of laboratory values, e.g., new or worsening hypercalcemia) indicating unequivocal progression of disease.
No decline in Eastern Cooperative Oncology Group (ECOG) Performance Status (e.g., see Table 3).
Absence of tumor progression at critical anatomical sites that cannot be readily managed and stabilized by protocol-allowed medical interventions prior to repeat dosing.
   Critical anatomical sites include the central nervous system (CNS), central airway, the great vessels, and other organs or tissues where compromised function secondary to tumor progression would be expected to result acutely in severe and/or irreversible disability or death.
Consent acknowledging discussion with the treating investigator of the benefit-risk balance of continuing study treatment beyond radiographic progression.

TABLE 3
Eastern Cooperative Oncology Group (ECOG) Performance Status

| Grade | Description |
|---|---|
| 0 | Fully active, able to carry on all predisease performance without restriction |
| 1 | Restricted in physically strenuous activity but ambulatory and able to carry |
| | out work of a light or sedentary nature; e.g., light housework or office work |
| 2 | Ambulatory and capable of all self-care but unable to carry out any work |
| | activities; up and about >50% of waking hours |
| 3 | Capable of only limited self-care, confined to a bed or chair >50% of |
| | waking hours |
| 4 | Completely disabled; cannot carry on any self-care; totally confined to |
| | bed or chair |
| 5 | Dead |

If radiographic disease progression is confirmed at a subsequent tumor assessment, patients who have received at least one prior systemic treatment for metastatic disease may be considered for continued study treatment if they continue to meet the criteria above and have evidence of clinical benefit, as evidenced by at least one of the following:
Tumor shrinkage (at least 30% decrease in diameter from baseline) of one or more evaluable lesions.
Improvement in one or more symptoms or signs attributable to the underlying cancer (e.g., decreased requirement for narcotics for pain, decreased dyspnea associated with pleural effusion, weight gain) as assessed by the investigator.

If radiographic disease progression is confirmed at a subsequent tumor assessment, patients who are receiving their first systemic therapy for metastatic disease may be considered for continued study treatment at the investigator's discretion after discussion with the Medical Monitor, if they continue to meet the criteria above and have evidence of clinical benefit, as evidenced by tumor shrinkage (at least 30% decrease in diameter from baseline) of one or more evaluable lesions.

Investigator assessment of overall tumor response at all timepoints may be based only on RECIST v1.1.

### C. Materials and Methods

i. Patients

### General Inclusion Criteria

Patients must meet the following criteria for study entry:
Age ≥18 years at time of signing Informed Consent Form
ECOG Performance Status of 0 or 1 (e.g., see Table 3)
Adequate hematologic and end-organ function, defined by the following laboratory test results, obtained within 14 days prior to initiation of study treatment:
   Absolute neutrophil count (ANC) without granulocyte colony-stimulating factor support Patients with NSCLC: ≥1.0×10⁹/L (≥1000/μL) Patients with GC and PDAC: ≥1.5×10⁹/L (≥1500/μL)
   Lymphocyte count 0.5×10⁹/L (≥500/μL)
   White blood cell (WBC) count ≥2.5×10⁹/L (≥2500/μL)
   Platelet count ≥100×10⁹/L (≥100,000/μL) without transfusion
   Hemoglobin 90 g/L (≥9 g/dL)
      Patients must not have been transfused within 2 weeks prior to screening hematology assessment to meet this criterion.
   Aspartate transaminase (AST), alanine transaminase (ALT), and alkaline phosphatase (ALP) ≤2.5×upper limit of normal (ULN), with the following exceptions:
      Patients with documented liver metastases: AST and ALT 5×ULN
      Patients with documented liver or bone metastases: ALP 5×ULN
   Total bilirubin ≤1.5×ULN, with the following exception:
      Patients with NSCLC: total bilirubin 1×ULN
   Creatinine clearance calculated through use of the Cockcroft-Gault formula:
      Patients with NSCLC: creatinine clearance ≥30 mL/min
      Patients with GC: creatinine clearance ≥60 mL/min
      Patients with PDAC: creatinine clearance ≥50 mL/min
   Albumin ≥25 g/L (≥2.5 g/dL)
   For patients not receiving therapeutic anticoagulation: international normalization ratio (INR) and activated partial thromboplastin time (aPTT)≤1.5×ULN
      Patients receiving therapeutic anticoagulation should be on a stable dose with INR in target range.
Histologically confirmed locally advanced, recurrent, or metastatic incurable solid tumor malignancy Additional cohort-specific criteria related to tumor type and prior lines of therapy are detailed below.
Measurable disease (at least one target lesion) according to RECIST v1.1 on computed tomography (CT) or magnetic resonance imaging (MRI) images within 28 days prior to enrollment.
   Previously irradiated lesions can be considered as measurable disease only if progressive disease has been unequivocally documented at that site since radiation.
Lesions that are intended to be biopsied must not count as target lesions.

### Cohort a (Non-Small Cell Lung Cancer) Inclusion Criteria

Patients in Cohort A also must meet the following criteria for study entry:
Histologically or cytologically confirmed metastatic nonsquamous or squamous NSCLC.
   Enrollment is managed such that approximately half of the accrued patients have squamous NSCLC.
Disease progression during or following treatment for metastatic or locally advanced, inoperable NSCLC that consisted of a platinum-containing regimen and a PD-L1/PD-1 CPI, given in combination for a maximum of one prior line of systemi therapy or as two separate lines of therapy (in either order), for a maximum of two prior lines of systemic therapy.
   Patients with disease progression or recurrence within 6 months after completion of their definitive therapy for locally advanced disease are allowed to participate, provided their treatment for locally advanced inoperable NSCLC included a combination of at least a platinum-based chemotherapy (e.g., cisplatin or carboplatin) and a PD-L1/PD-1 CPI.
   Prior exposure to anti-cytotoxic T-lymphocyte-associated protein 4 (CTLA-4) antibodies is allowed if used in combination with a PD-L1/PD-1 CPI.
Positive tumor PD-L1 status (e.g., ≥1%), as determined by a local laboratory through use of a health authority-approved or CE-marked assay.

### Cohort B (Gastric Cancer) Inclusion Criteria

Patients in Cohort B must also meet the following criteria for study entry:
   Unresectable locally advanced or metastatic GC (including esophagogastric junction cancer) that is histologically confirmed to be adenocarcinoma.
   Cohort C (Pancreatic Ductal Adenocarcinoma) Inclusion Criteria
Patients in Cohort C must also meet the following criteria for study entry:
   Histologically or cytologically confirmed metastatic PDAC. The definitive diagnosis of metastatic PDAC may be made, e.g., by evaluating the histopathologic data within the context of clinical and/or radiographic data.

### General Exclusion Criteria

Patients who meet any of the following criteria are excluded from study entry:
Any anti-cancer therapy, whether investigational or approved, including chemotherapy, hormonal therapy, and/or radiotherapy, within 3 weeks prior to initiation of study treatment, with the following exceptions:
   Hormone-replacement therapy or oral contraceptives
   Herbal therapy intended for the treatment of cancer >7 days before Day 1 of Cycle 1
   Palliative radiotherapy for painful metastases or metastases in potentially sensitive locations (e.g., epidural space) >2 weeks prior to Day 1 of Cycle 1.
Treatment with cancer vaccines within 6 weeks or 5 drug elimination half-lives (whichever is shorter) prior to initiation of study treatment.
Treatment with systemic immunostimulatory agents (including, but not limited to, interferons (IFNs) and IL-2) within 4 weeks or 5 drug-elimination half-lives (whichever is longer) prior to initiation of study treatment and during study treatment.
Prior treatment with TGF-β or TGF-β receptor inhibitor (e.g., bintrafusp alpha, SRK-181, NIS793, and the like).
Symptomatic, untreated, or actively progressing CNS metastases.
   Asymptomatic patients with treated CNS lesions are eligible, provided that all of the following criteria are met:
      Measurable disease per RECIST v1.1 must be present outside the CNS.
      No evidence of acute or subacute brain metastasis-related hemorrhage was seen on CNS imaging at screening.
      The patient has not undergone stereotactic radiotherapy within 7 days prior to initiation of study treatment, whole-brain radiotherapy within 14 days prior to initiation of study treatment, or neurosurgical resection within 28 days prior to initiation of study treatment.
      The patient has no ongoing requirement for corticosteroids as therapy for CNS disease, with corticosteroids discontinued for ≥2 weeks prior to enrollment. Anticonvulsant therapy at a stable dose is permitted.
      There is no evidence of interim progression between completion of CNS-directed therapy and initiation of study treatment.
      Metastases are limited to the cerebellum or the supratentorial region (i.e., no metastases to the midbrain, pons, medulla, or spinal cord).
      Note: Asymptomatic patients with CNS metastases newly detected at screening are eligible for the study after receiving radiotherapy or surgery, with no need to repeat the screening brain scan.
Untreated keratoacanthoma
   Patients who have undergone successful treatment for keratoacanthoma (i.e., surgical resection or other appropriate procedure) in the investigator's judgment prior to initiation of study treatment are eligible.
History of leptomeningeal disease.
Spinal cord compression not definitively treated with surgery and/or radiation, or previously diagnosed and treated spinal cord compression without evidence that disease has been clinically stable for ≥2 weeks prior to screening.
Significant cardiovascular disease (such as New York Heart Association Class II or greater cardiac disease, myocardial infarction, or cerebrovascular accident) within 3 months prior to initiation of study treatment, unstable arrhythmia, or unstable angina.
Any disease, metabolic dysfunction, physical examination finding, or clinical laboratory finding that contraindicates the use of an investigational drug, may affect the interpretation of the results, or may render the patient at high risk from treatment complications, including, but not limited to:
   Known clinically significant liver disease, including active viral, alcoholic, or other hepatitis, cirrhosis, and inherited liver disease or current alcohol abuse
   Severe dyspnea or requirement for supplemental oxygen at rest
   History of Stevens-Johnson syndrome, toxic epidermal necrolysis, or drug rash with eosinophilia and systemic symptoms.
Uncontrolled tumor-related pain
   Patients requiring pain medication must be on a stable regimen at study entry.
   Symptomatic lesions (e.g., bone metastases or metastases causing nerve impingement) amenable to palliative radiotherapy should be treated prior to enrollment. Patients should be recovered from the effects of radiation.
   Asymptomatic metastatic lesions that would likely cause functional deficits or intractable pain with further growth (e.g., epidural metastasis that is not currently associated with spinal cord compression) should be considered for loco-regional therapy, if appropriate, prior to enrollment.
Uncontrolled pleural effusion, pericardial effusion, or ascites requiring recurrent drainage procedures (once monthly or more frequently).
   Patients with indwelling catheters (e.g., PLEURX®) are eligible.
Uncontrolled or symptomatic hypercalcemia (ionized calcium >1.5 mmol/L, calcium >12 mg/dL, or corrected calcium >ULN).
History of malignancy other than the disease under study within 3 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer.
Adverse events from prior anti-cancer therapy (with the exception of immune-related adverse events attributed to cancer immunotherapy; see below) that have not resolved to Grade 1 except for alopecia, vitiligo, or endocrinopathy managed with replacement therapy.
Any history of an immune-mediated Grade 4 adverse event attributed to prior cancer immunotherapy (other than endocrinopathy managed with replacement therapy or asymptomatic elevation of serum amylase and/or lipase).
Any history of an immune-mediated Grade 3 adverse event attributed to prior cancer immunotherapy (other than endocrinopathy managed with replacement therapy or asymptomatic elevation of serum amylase and/or lipase) that resulted in permanent discontinuation of the prior immunotherapeutic agent and/or occurred 6 months prior to planned Day 1 of Cycle 1.
Any immune-mediated adverse event related to prior cancer immunotherapy (other than endocrinopathy managed with replacement therapy or stable vitiligo) that has not resolved completely to baseline.
   Patients treated with corticosteroids for immune-mediated adverse events must demonstrate absence of related symptoms or signs for ≥4 weeks following discontinuation of corticosteroids.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions:
   Patients with a history of autoimmune-mediated hypothyroidism who are on thyroid-replacement hormone are eligible for the study.
   Patients with controlled Type 1 diabetes mellitus who are on an insulin regimen are eligible for the study.
   Patients with eczema, psoriasis, lichen simplex chronicus, or vitiligo with dermatologic manifestations only (e.g., patients with psoriatic arthritis are excluded) are eligible for the study, provided all of following conditions are met:
      Rash must cover <10% of body surface area.
      Disease is well controlled at baseline and requires only low-potency topical corticosteroids.
      No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest CT scan.
   History of radiation pneumonitis in the radiation field (fibrosis) is permitted.
Active tuberculosis.
Severe infection within 4 weeks prior to initiation of study treatment, including, but not limited to, hospitalization for complications of infection, bacteremia, or severe pneumonia.
Treatment with IV antibiotics within 2 weeks prior to initiation of study treatment.
   Patients receiving prophylactic antibiotics (e.g., to prevent a urinary tract infection or chronic obstructive pulmonary disease (COPD) exacerbation) are eligible for the study.
Positive test for HIV infection.
Positive hepatitis B surface antigen (HbsAg) test, and/or positive total hepatitis B core antibody (HbcAb) test at screening.
   Patients with positive total HbcAb test followed by a negative hepatitis B virus (HBV) DNA test at screening can be enrolled.
   Antiviral prophylaxis for patients at risk for HBV reactivation is permitted.
Positive hepatitis C virus (HCV) antibody test at screening.
   Patients positive for HCV antibody are eligible only if PCR is negative for HCV RNA.
Known infection with SARS-CoV-2 (the virus that causes COVID-19), persistent symptoms of known prior SARS-CoV-2 infection, and/or known positive SARS-CoV-2 test within 4 weeks prior to screening.
Administration of a live, attenuated vaccine (e.g., FLUMIST®) within 4 weeks before first study treatment or anticipation that such a live, attenuated vaccine may be required during the study or within 5 months after the final dose of study treatment.
Treatment with systemic immunosuppressive medication (including, but not limited to, corticosteroids, cyclophosphamide, azathioprine, methotrexate, thalidomide, and anti-tumor necrosis factor (TNF) agents) within 2 weeks prior to initiation of study treatment, or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) are eligible for the study.
   Patients who received mineralocorticoids (e.g., fludrocortisone), inhaled or low-dose corticosteroids for COPD or asthma, or low-dose corticosteroids for orthostatic hypotension or adrenal insufficiency are eligible for the study.
Major surgical procedure or significant traumatic injury within 28 days prior to first study treatment, or anticipation of the need for major surgery before the end of the treatment period.
   After major surgery, the patient must wait until surgical wounds are fully healed before initiating treatment.
Prior allogeneic stem cell or organ transplantation.
History of severe allergic anaphylactic reactions to chimeric or humanized antibodies or fusion proteins.
Known allergy or hypersensitivity to Chinese hamster ovary cell products, any component of the SOF10 formulation, or any of the study drugs or their excipients.

### Cohort A (Non-Small Cell Lung Cancer) Exclusion Criteria

Patients in Cohort A who also meet any of the following criteria are excluded from study entry:
Activating mutations in the EGFR gene or ALKgene rearrangement
   Patients with unknown EGFR and/or ALK status may undergo testing at a local laboratory at screening, with the exception of patients with squamous NSCLC.
Documented additional actionable driver mutations, such as KRAS, G12C, ROS1, BRAFV600E, NTRK, METex14, and RET unless the targeted therapies are not available or not appropriate per investigator discretion.
Prior treatment with a T-cell co-stimulating therapy or an immune CPI other than a PD-L1/PD-1 CPI and anti-CTLA-4 antibodies.
Discontinuation of a PD-L1/PD-1 CPI primarily for toxicity or intolerability.
Known hypersensitivity to any component of the atezolizumab formulation.

### Cohort B (Gastric Cancer) Exclusion Criteria

Patients in Cohort B who also meet any of the following criteria are excluded from study entry:
Known HER2-positive status.
   Determination for positive is made on the basis of the reference at each site. If there is no reference, rough indication for positive is 3+ by IHC, or 2+ by IHC and positive by ISH.
Prior systemic treatment for GC.
Prior treatment with first-line therapy with systemic anti-tumor agents for advanced or recurrent GC
   Patients who experience disease progression more than 6 months after the last dose of adjuvant and/or neoadjuvant therapy are eligible.
Prior treatment with T-cell co-stimulating or immune checkpoint blockade therapies, including anti-CTLA-4, anti-PD-1, and anti-PD-L1 therapeutic antibodies.
Grade ≥2 peripheral neuropathy as defined by the CTCAE v5.0.
Inability to swallow medication or malabsorption condition that would alter the absorption of orally administered medications.
Known hypersensitivity to any component of the nivolumab formulation.
Any contraindications to any of the study drugs of the chemotherapy regimen (capecitabine plus oxaliplatin or S-1 plus oxaliplatin) selected by the investigator.

### Cohort C (Pancreatic Ductal Adenocarcinoma) Exclusion Criteria

Patients in Cohort C who also meet any of the following criteria are excluded from study entry:
Prior systemic treatment for unresectable PDAC.
Prior treatment with a T-cell co-stimulating therapy or a CPI including anti-cytotoxic T-lymphocyte-associated antigen 4 (CTLA-4), anti-PD-1, and anti-PD-L1 therapeutic antibodies.
Grade ≥2 peripheral neuropathy as defined by the CTCAE v5.0.
Known hypersensitivity to any component of the atezolizumab formulation.
Any contraindications to gemcitabine or nab-paclitaxel.
   ii. Study Treatment and Other Treatments Relevant to the Study Design

The investigational medicinal products (IMPs) for this study are SOF10, atezolizumab, nivolumab, gemcitabine, nab-paclitaxel, capecitabine, oxaliplatin, and S-1.

### Standard-of-Care (SOC) Treatments

SOC treatments (nivolumab, gemcitabine, nab-paclitaxel, capecitabine, oxaliplatin, and S-1) are supplied when required by local health authority regulations. For information on the formulation and packaging of SOC treatments, refer to the local prescribing information for each SOC treatment.

### Study Treatment Dosage, Administration, and Compliance

The investigational medicinal products (IMPs) for this study are SOF10, atezolizumab, nivolumab, gemcitabine, nab-paclitaxel, capecitabine, oxaliplatin, and S-1. Patients receive SOF10 in combination with a checkpoint inhibitor (Cohort A); or SOF10 in combination with a checkpoint inhibitor and standard-of-care chemotherapy (Cohorts B and C) (e.g., see Table 4). Patients receive study treatment in 21-day cycles (Cohorts A and B) or 28-day cycles (Cohort C). The treatment regimens are summarized in Table 4 below, with each treatment listed in order of administration.

TABLE 4
Treatment Regimens

| | Treatment | Cycle | Dose, Route, and Regimen |
|---|---|---|---|
| Cohort | Regimen | Length | (drugs listed in order of administration) |
| Cohort A: | SOF10 and | 21 | SOF10 1800 mg IV on Day 1 of each |
| Non-small cell | atezolizumab | Days | cycle |
| lung cancer | | | Atezolizumab 1200 mg IV on Day 1 of |
| | | | each cycle |
| Cohort B: | SOF10, | 21 | Capecitabine or S-1 ^{b} |
| Gastric cancer | nivolumab, | Days | Capecitabine 1000 mg/m² PO |
| | oxaliplatin, and | | twice daily on Days 1-14 ^{c} |
| | capecitabine or | | S-1 40 mg/m² PO twice daily on Days |
| | S-1 | | 1-14 ^{d} |
| | | | SOF10 1800 mg IV on Day 1 of each |
| | | | cycle |
| | | | Nivolumab 360 mg IV on Day 1 of |
| | | | each cycle |
| | | | Oxaliplatin 130 mg/m² IV on Day 1 of |
| | | | each cycle |
| Cohort C: | SOF10, | 28 | SOF10 1200 mg IV on Days 1 and 15 |
| Pancreatic ductal | atezolizumab, | Days | of each cycle |
| adenocarcinoma | nab-paclitaxel, | | Atezolizumab 840 mg IV on Days 1 |
| | and | | and 15 of each cycle |
| | gemcitabine | | Nab-paclitaxel 125 mg/m² IV on |
| | | | Days 1, 8, and 15 of each cycle |
| | | | Gemcitabine 1000 mg/m² IV on Days |
| | | | 1, 8, and 15 of each cycle |

BSA = body surface area; nab-paclitaxel = nanoparticle albumin-bound paclitaxel; PO = by mouth; S-1 = tegafur-gimeracil-oteracil potassium.
^{b} For Cohort B, either capecitabine or S-1 may be used at the investigator's discretion. Capecitabine or S-1 may be administered prior to or after SOF10, nivolumab, and oxaliplatin infusions at the investigator's discretion.
^{c} Capecitabine should be taken with water and within 30 minutes after a meal.
^{d} S-1 is administered twice a day at 40 mg/dose for body surface area (BSA) <1.25 m², 50 mg/dose for BSA ≥1.25 m² and <1.5 m², and 60 mg/dose for BSA ≥1.5 m². S-1 should be taken with water at least 60 minutes before or after a meal.

### SOF10

The SOF10 drug product is supplied as a sterile liquid in a single-use, 20-ml glass vial. The vial contains approximately 10 mL (600 mg) of SOF10 solution.

An integrated approach based on the totality of nonclinical data, clinical pharmacology data, and preliminary Phase I clinical data from an ongoing phase I study has been used to select the initial SOF10 dose for this study, which is 1800 mg Q3W. For the NSCLC and GC cohorts, SOF10 is administered by IV infusion at a fixed dose of 1800 mg on Day 1 of each 21-day cycle in combination with atezolizumab or nivolumab and SOC chemotherapy. For the PDAC cohort, SOF10 is administered by IV infusion at a fixed dose of 1200 mg on Day 1 and Day 15 of each 28-day cycle in combination with atezolizumab and SOC chemotherapy. The dose level of SOF10 is based on the totality of data from the ongoing phase I study, and may be updated once additional data from the ongoing phase I study becomes available.

The initial dose of SOF10 is delivered over 60 (±10) minutes (although the infusion may be slowed or interrupted for patients who experience infusion-associated symptoms), followed by a 60-minute observation period prior to dosing with atezolizumab or nivolumab. If the 60-minute infusion is tolerated without infusion-associated adverse events (e.g., see Table 5), the second infusion may be delivered over 30 (±10) minutes, followed by a 30-minute observation period (e.g., see Table 6).

Guidance on study drug administration in the context of management of specific adverse events is provided in Table 5.

TABLE 5
Management Guidelines for Specific Adverse Events Associated with SOF10

| | |
|---|---|
| Event | Management |

Cardiovascular Toxicity

| | |
|---|---|
| Cardiovascular toxicity, | Discontinue SOF10. ^{a} |
| Grade 2 | |
| Cardiovascular toxicity, | Permanently discontinue SOF10. |
| Grade 3 or 4 | |

Keratoacanthoma/Squamous Cell Carcinoma

| | |
|---|---|
| Any grade | Discontinue SOF10. |
| Keratoacanthoma/ | Atezolizumab or nivolumab may be continued. |
| squamous cell carcinoma | Consult dermatologist for treatment and/or surgical removal. |
| | SOF10 may be resumed after surgical removal. ^{a} |
| | Periodic skin evaluations should be implemented for recurrence. |

Hemorrhage

| | |
|---|---|
| Hemorrhage event, Grade | If hemorrhage occurs when anticoagulants are administered or |
| 2 | surgery is required during the course of the study (excluding |
| | biopsies established in this study), discontinue SOF10. ^{a} |
| | Atezolizumab or nivolumab may be continued. |
| Hemorrhage event, Grade | Discontinue SOF10. ^{a} |
| 3 or 4 | Atezolizumab or nivolumab may be continued. |

Hypersensitivity and Infusion-Related Reactions

| | |
|---|---|
| Infusion-related reactions | Administration of SOF10 and/or atezolizumab is managed according |
| | to the guidance for the management of infusion-related reactions |
| | and cytokine release syndrome. |

^{a} If administration of SOF10 cannot be resumed for more than 42 days after the previous administration, permanently discontinue SOF10.

TABLE 6
Administration of First and Subsequent Infusions of SOF10

| First Infusion | Subsequent Infusions |
|---|---|
| No premedication is indicated prior to the | If the patient experienced an |
| SOF10 infusion. | infusion-related reaction with any |
| Vital signs (pulse rate, respiratory rate, blood | previous infusion, premedication with |
| pressure, and temperature) should be | antihistamines, antipyretics, and/or |
| measured within 60 minutes prior to the | analgesics are administered for |
| infusion. | subsequent doses at the discretion of the |
| SOF10 should be infused over | investigator. |
| 60 (±10) minutes. | Vital signs may be measured within |
| Patients should be observed for 60 minutes | 60 minutes prior to the infusions. |
| after the first infusion prior to dosing with | SOF10 may be infused over 30 (±10) |
| atezolizumab or nivolumab. ^{a} | minutes if the previous 60-minute infusion |
| Vital signs should be measured every | was tolerated without an infusion-related |
| 30 (±10) minutes during the infusion and at | reaction. |
| 30 (±10) and 60 (±10) minutes after the end of | If the patient experienced an infusion- |
| infusion. | related reaction with the previous |
| Patients should be informed about the | infusion, SOF10 should be infused over |
| possibility of delayed post-infusion symptoms | the same duration as the previous |
| and instructed to contact their study physician if | infusion, or longer per investigator's |
| they develop such symptoms. | discretion. |
| | Patients are observed for 30 minutes |
| | after 30-minute infusions and prior to |
| | dosing with atezolizumab or nivolumab. |
| | If the patient experienced an |
| | infusion-related reaction with the previous |
| | infusion or if clinically indicated, vital |
| | signs should be measured every 30 |
| | (±10) minutes during the infusion, and at |
| | least 30 (±10) minutes after the infusion. |
| | Additional vital signs are collected as |
| | clinically indicated. |

^{a} Patients may be hospitalized overnight if required by institutional guidelines. If infusion-related adverse events (e.g., fever, chills) are not resolved to Grade ≤1 following the outpatient observation period, patients should be admitted for overnight observation.

### Atezolizumab

The atezolizumab drug product is supplied as a sterile liquid in a single-use, 15-ml or 20-mL glass vial. The vial contains approximately 14 mL (840 mg) or 20 mL (1200 mg) of atezolizumab solution.

For the PDAC cohort, atezolizumab is administered by IV infusion at a fixed dose of 840 mg on Day 1 and Day 15 of each 28-day cycle in combination with SOF10 and SOC chemotherapy. For the NSCLC cohort, atezolizumab is administered by IV infusion at a fixed dose of 1200 mg on Day 1 of each 21-day cycle in combination with SOF10. Atezolizumab is administered after SOF10.

Exemplary instructions for vital sign monitoring are provided in Table 7 below.

Atezolizumab infusions are administered per the instructions outlined in Table 7, including for patients who are administered atezolizumab alone (e.g., due to adverse events associated with SOF10). Administration of atezolizumab may be performed in a monitored setting where there is immediate access to trained personnel and adequate equipment and medicine to manage potentially serious reactions. Guidelines for medical management of infusion-related reactions (IRRs) are provided in.

TABLE 7
Administration of First and Subsequent Infusions of Atezolizumab

| First Infusion | Subsequent Infusions |
|---|---|
| Unless indicated, no premedication is given | If the patient experienced an IRR with any |
| prior to the atezolizumab infusion. | previous infusion, premedication with |
| Vital signs (pulse rate, respiratory rate, blood | antihistamines, anti-pyretic medications, |
| pressure, and temperature) should be | and/or analgesics may be administered for |
| measured within 60 minutes prior to the | subsequent doses at the discretion of the |
| infusion. | investigator. |
| Atezolizumab should be infused over | Vital signs should be measured within |
| 60 (±15) minutes. | 60 minutes prior to the infusion. |
| If clinically indicated, vital signs should be | Atezolizumab should be infused over |
| measured every 15 (±5) minutes during the | 30 (±10) minutes if the previous infusion was |
| infusion and at 30 (±10) minutes after the | tolerated without an IRR, or |
| infusion. | 60 (±15) minutes if the patient experienced |
| Patients should be informed about the | an IRR with the previous infusion. |
| possibility of delayed post-infusion symptoms | If the patient experienced an IRR with the |
| and instructed to contact their study | previous infusion or if clinically indicated, vital |
| physician if they develop such symptoms. | signs should be measured during the infusion |
| | and at 30 (±10) minutes after the infusion. |

IRR = infusion-related reaction.

### Nivolumab

For the GC cohort, nivolumab is administered at a dose of 360 mg as an IV infusion over 30 (±10) minutes. Nivolumab is administered after SOF10. For guidance on nivolumab administration in the context of management of specific adverse events, refer to the local prescribing information.

### Standard-of-Care Chemotherapy for the Gastric Cancer Cohort (Cohort B)

Patients in the GC cohort receive SOC chemotherapy (oxaliplatin in combination with either capecitabine or S-1) in combination with SOF10 and nivolumab as part of the treatment regimens outlined in Table 4. SOC chemotherapy may be administered per local prescribing information. Patients may receive either capecitabine or S-1. S-1 may be administered only at sites that can produce drug supply for patients from local sources. The same SOC regimen (e.g., oxaliplatin and capecitabine or oxaliplatin and S-1) may be administered for the duration of the study treatment period.

Oxaliplatin 130 mg/m² is administered by IV infusion over approximately 2 hours on Day 1 of each 21-day cycle after completion of the nivolumab infusion.

Capecitabine 1000 mg/m² or S-1 40 mg/m² (40 mg/dose for body surface area (BSA)<1.25 m², 50 mg/dose for BSA between ≥1.25 m² and <1.5 m², and 60 mg/dose for BSA≥1.5 m²) is administered orally twice a day for 14 days (Days 1-14 of each cycle) followed by a 7-day rest period. Capecitabine or S-1 may be administered prior to or after the SOF10, nivolumab, and oxaliplatin infusions. The first dose of capecitabine or S-1 should be administered on the morning of Day 1 and the last dose on the evening of Day 14. Alternatively, the first dose of capecitabine may be administered on the evening of Day 1 an the last dose on the morning of Day 15. Capecitabine is taken orally with water and within 30 minutes after a meal. S-1 is taken orally with water at least 60 minutes before or after a meal.

Oxaliplatin, capecitabine, and S-1 may be administered according to local prescribing information in a monitored setting where there is immediate access to trained personnel and adequate equipment and medicine to manage potentially serious reactions. Oxaliplatin, capecitabine, and S-1 treatment may be interrupted for reasons other than toxicity (e.g., surgical procedures). Patients may receive prophylactic supportive medications per institutional guidelines. Corticosteroids may be used in the usual dosage and mode of administration in the site for patients in whom chemotherapy-induced nausea/vomiting is concerned.

### Standard-of-Care Chemotherapy for the Pancreatic Ductal Adenocarcinoma Cohort (Cohort C)

Patients in the PDAC cohort receive SOC chemotherapy (nab-paclitaxel in combination with gemcitabine) in combination with SOF10 and atezolizumab as part of the treatment regimens outlined in, e.g., Table 4. Nab-paclitaxel and gemcitabine may be administered per local prescribing information.

On Days 1, 8, and 15, patients receive nab-paclitaxel 125 mg/m² administered by IV infusion over 30 (±5) minutes, followed by gemcitabine 1000 mg/m² administered by IV infusion over 30 (±5) minutes. On Days 1 and 15 of each cycle, nab-paclitaxel is administered after completion of the atezolizumab infusion.

iii. Concomitant Therapy

Concomitant therapy consists of any medication (e.g., prescription drugs, over-the-counter drugs, vaccines, herbal or homeopathic remedies, nutritional supplements) used by a patient in addition to protocol-mandated treatment from 7 days prior to initiation of study treatment to the treatment discontinuation visit. All such medications should be reported to the investigator and recorded on the Concomitant Medication eCRF.

### Permitted Therapy

Patients are permitted to use the following therapies during the study:
Oral contraceptives with a failure rate of <1% per year
Hormone-replacement therapy
Vaccinations (such as influenza, COVID-19)
Megestrol acetate administered as an appetite stimulant
Mineralocorticoids (e.g., fludrocortisone)
Inhaled or low-dose corticosteroids administered for chronic obstructive pulmonary disease (COPD) or asthma
Low-dose corticosteroids administered for orthostatic hypotension, or adrenocortical insufficiency
Corticosteroids administered for chemotherapy-induced nausea/vomiting
Palliative radiotherapy (e.g., treatment of known bony metastases or symptomatic relief of pain) as outlined below:
   Palliative radiotherapy is permitted if patients are otherwise deriving benefit. Study treatment may be continued or suspended during palliative radiotherapy and should be discussed with the investigator in consultation with the Medical Monitor.

Premedication with antihistamines, anti-pyretic medications, and/or analgesics may be administered for the second and subsequent SOF10 or atezolizumab infusions only. Patients who experience infusion-associated symptoms may be treated symptomatically with acetaminophen, ibuprofen, diphenhydramine, and/or H2-receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β₂-adrenergic agonists).

### Corticosteroids, Immunosuppressive Medications, and TNF-α Inhibitors

Systemic corticosteroids, immunosuppressive medications, and TNF-α inhibitors may attenuate potential beneficial immunologic effects of treatment with SOF10 in combination with CPIs. Therefore, in situations in which systemic corticosteroids, immunosuppressive medications, or TNF-α inhibitors would be routinely administered, alternatives, including antihistamines, should be considered, except when systemically administered for orthostatic hypotension or adrenocortical insufficiency or chemotherapy-induced nausea/vomiting. If the alternatives are not feasible, systemic corticosteroids, immunosuppressive medications, and TNF-α inhibitors may be administered. Systemic corticosteroids or immunosuppressive medications are recommended, at the discretion of the investigator, for the treatment of specific adverse events when associated with SOF10 and/or CPI therapy.

Medications Given with Precaution Due to Effects Related to Cytochrome P450 Enzymes

The metabolism of nab-paclitaxel is catalyzed by cytochrome (CYP) isoenzymes CYP2C8 and CYP3A4. Caution should be exercised when nab-paclitaxel is concomitantly administered with known CYP2C8 or CYP3A4 inhibitors (e.g., atazanavir, clarithromycin, indinavir, itraconazole, ketoconazole, nefazodone, nelfinavir, ritonavir, saquinavir, and telithromycin) and inducers (e.g., rifampin and carbamazepine). Because CYP2A6 is the major enzyme responsible for the conversion of tegafur to 5-FU, co-administration of a known CYP2A6 inhibitor (e.g., letrozole, clotrimazole, tranylcypromine, pilocarpine, miconazole) and S-1 should be avoided, as the effectiveness of S-1 could be decreased. Refer to the local prescribing information for nab-paclitaxel and S-1 for all boxed warnings and contraindications.

### Anticoagulation Therapies

Use of prophylactic or therapeutic anticoagulation therapies (such as warfarin at a stable dose or low-molecular-weight heparin) may be used.

### Vital Signs

Vital signs may include measurements of respiratory rate, pulse rate, systolic and diastolic blood pressure while the patient is in a seated position, and temperature. Blood oxygen saturation may be measured at baseline and as clinically indicated by pulse oximetry. Vital signs for patients may be obtained according to Table 6 and Table 7.

### Tumor and Response Evaluations

Patients may undergo tumor assessments at baseline and following initiation of study treatment (e.g., see Table 8 for details), regardless of dose delays, until radiographic disease progression according to RECIST v1.1. Thus, tumor assessments are to continue according to schedule in patients who discontinue treatment for reasons other than disease progression or loss of clinical benefit. Patients who continue treatment after radiographic disease progression may undergo tumor assessments every 6 weeks (±1 week) until loss of clinical benefit, as determined by the investigator (e.g., a clinician). Tumor assessments may be continued every 6 weeks thereafter until two consecutive scans demonstrate stability or improvement with respect to the first scan that showed radiographic disease progression, at which point the scan frequency can revert to the initial schedule (e.g., see Table 8). At the investigator's discretion, tumor assessments may be repeated at any time if progressive disease is suspected.

TABLE 8
Tumor Assessment Frequency for Each Cohort

| Cohort | Tumor Assessment Frequency |
|---|---|
| NSCLC (Cohort A) | Every 6 weeks (±1 week) for the first 48 weeks |
| GC (Cohort B) | Every 12 weeks (±2 weeks) thereafter |
| PDAC (Cohort C) | Every 8 weeks (±1 week) for the first 48 weeks |
| | Every 12 weeks (±2 weeks) thereafter |

Screening assessments may include CT scans (with IV contrast, unless contraindicated, and oral contrast as appropriate per institutional standards) of the chest, abdomen, and pelvis. CT scans of the abdomen and pelvis may be substituted by MRI scans. A CT scan with contrast or MRI scan of the head must be performed at screening for patients with known or clinically suspected brain metastases or treated brain metastases. In the event of an equivocal CT scan at screening, an MRI scan of the brain is required to confirm or refute the diagnosis of CNS metastases. Subsequent brain scans should be performed only if clinically indicated (e.g., if a patient becomes symptomatic). Bone scans and CT scans of the neck should also be performed if clinically indicated. At the investigator's discretion, other methods of assessment of measurable disease per RECIST v1.1 may be used. If a CT scan for tumor assessment is performed in a positron emission tomography-CT scanner, the CT acquisition must be consistent with the standards for a full-contrast diagnostic CT scan.

All measurable and/or evaluable lesions identified at baseline should be re-assessed at subsequent tumor evaluations according to the schedules described above (e.g., see Table 8). The same radiographic procedures used to assess disease sites at screening should be used for subsequent tumor assessments (e.g., the same contrast protocol for CT scans). To facilitate evaluation of post-progression tumor changes while treatment is ongoing, tumor assessments must be continued after disease progression per RECIST v1.1 for patients who receive treatment beyond progression. This includes continued measurement of target lesions, evaluation of non-target lesions (including monitoring for further worsening of any non-target lesions that have shown unequivocal progression), and evaluation of any newly identified lesions (including measurements, if lesions are measurable) at all subsequent assessments.

Overall response at a single timepoint is assessed by the investigator using RECIST v1.1. An objective response should be confirmed by repeat assessments ≥4 weeks after initial documentation.

Assessments should be performed by the same evaluator, if possible, to ensure internal consistency across visits. Available results must be reviewed by the investigator prior to treatment administration.

### Laboratory and Other Biological Samples

Samples for the following laboratory tests may be sent to the study site's local laboratory for analysis:
Hematology: WBC count, RBC count, hemoglobin, hematocrit, platelet count, and differential count (neutrophils, eosinophils, basophils, monocytes, lymphocytes, other cells (if indicated))
Chemistry panel (serum or plasma): bicarbonate or total carbon dioxide (if considered SOC for the region), sodium, potassium, chloride, glucose, BUN or urea, creatinine, total protein, albumin, phosphate, calcium, total bilirubin (direct bilirubin if total is abnormal), ALP, ALT, AST, and LDH
Coagulation: INR, aPTT, and PT
Amylase and lipase
Thyroid function testing: thyroid-stimulating hormone, free T3 (or total T3 for sites where free T3 is not performed), and free T4
HIV serology: HIV-1/2 antibody or HIV-1 and HIV-2 antibodies, unless not permitted per local regulations
HBV serology: HBsAg, hepatitis B surface antibody (HBsAb), and total HBcAb for all patients; HBV DNA for patients with negative HBsAg and HBsAb tests and a positive total HBcAb test
HCV serology: HCV antibody for all patients; HCV RNA for patients with a positive HCV antibody test
   If a patient has a positive HCV antibody test at screening, an HCV RNA test must also be performed to determine if the patient has an HCV infection.
Tuberculosis testing (only if patient is considered at increased risk for infection with Mycobacterium tuberculosis): IFN-γ release assays or tuberculin skin test (according to local standard practice)
C-reactive protein and serum ferritin
Carbohydrate antigen 19-9 (e.g., for GC and PDAC cohorts only)

### Electrocardiograms (ECGs)

Triplicate 12-lead ECG recordings may be obtained at specified timepoints, as outlined in the schedule of activities. ECGs acquired on different days should be as closely time-matched as feasible. Three interpretable ECG recordings (e.g., without artifacts) may be obtained at each timepoint (±10 minutes). The average of the three readings may be used to determine ECG intervals (e.g., PR, QRS, QT). Single ECG recordings may be obtained at unscheduled timepoints as clinically indicated. ECG recordings may be performed after the patient has been resting in a supine or semi-supine position for at least 10 minutes, and the patient should remain in a supine or semi-supine position during recording. Triplicate ECGs must be performed within 5-10 minutes of each other. The same positioning should be maintained for each patient throughout the study. ECG recordings should be performed prior to other procedures scheduled at that same time (e.g., vital sign measurements, blood draws) and should not be performed within 2.5 hours after any meal. Circumstances that may induce changes in heart rate, including environmental distractions (e.g., television, radio, conversation), should be avoided during the pre-ECG resting period and during ECG recording.

### Optional Tumor Biopsies

Consenting patients may undergo an optional biopsy at any time at the investigator's discretion (if deemed clinically feasible by the investigator), such as at time of disease progression. For patients with confirmed, prolonged CR and/or PR (e.g., approximately 1 year in duration) who have an accessible residual mass, a biopsy of that residual mass is recommended to assess for viable tumor cells (vs. fibrotic or necrotic tissue). Samples collected via resection, core-needle biopsy (at least three cores preferred), or excisional, incisional, punch, or forceps biopsy are preferred.

### Example 2. Urothelial Carcinoma Substudy

This example describes a substudy of the clinical trial described in Example 1 that is directed to treatment of urothelial carcinoma with anti-latent TGF-beta 1 antibodies as disclosed herein, e.g., SOF10, in combination with a checkpoint inhibitor (e.g., an anti-PD-L1 antibody such as atezolizumab).

### A. Objectives and Endpoints

The present example is a urothelial carcinoma (UC) substudy of the clinical study described in Example 1 that evaluates the safety, tolerability, pharmacokinetics, immunogenicity, and preliminary anti-tumor activity of SOF10 when administered in combination with atezolizumab in patients with urothelial carcinoma.

In this protocol, “study treatment” refers to the combination of all treatments assigned to patients as part of this study (e.g., SOF10 in combination with atezolizumab).

Specific objectives and corresponding endpoints for the substudy are outlined below in Table 9.

TABLE 9
Objectives and Corresponding Endpoints for Urothelial Carcinoma Substudy

| | Corresponding Endpoints |
|---|---|
| Primary Objective | |
| To evaluate the safety and tolerability of | Incidence and severity of adverse events, with |
| SOF10 when administered in | severity determined according to the CTCAE v5.0 |
| combination with atezolizumab | grading scale |
| | Change from baseline in targeted vital signs |
| | Change from baseline in targeted clinical |
| | laboratory test results |
| Secondary Objectives | |
| To characterize the pharmacokinetics of | Plasma concentration of SOF10 at specified |
| SOF10 and atezolizumab | timepoints and relevant PK parameters |
| | Serum concentration of atezolizumab at |
| | specified timepoints and relevant PK |
| | parameters |
| To evaluate the immune response to | Prevalence of ADAs to SOF10 and |
| SOF10 and atezolizumab | atezolizumab at baseline and incidence of |
| | ADAs to SOF10 and atezolizumab during the |
| | study |
| To make a preliminary assessment of the | Confirmed ORR, defined as the proportion of |
| activity of SOF10 when administered in | patients with a CR or PR on two consecutive |
| combination with atezolizumab | occasions ≥4 weeks apart, as determined by the |
| | investigator according to RECIST v1.1 |
| | DOR, defined as the time from the first occurrence |
| | of a documented confirmed objective response to |
| | disease progression or death from any cause |
| | during the study (whichever occurs first), as |
| | determined by the investigator according to |
| | RECIST v1.1 |
| | PFS, defined as the time from the first SOF10 |
| | treatment day to the first occurrence of disease |
| | progression or death from any cause during the |
| | study (whichever occurs first), as determined by the |
| | investigator according to RECIST v1.1 |
| To identify a recommended dose and regimen | Relationship between SOF10 dose and safety, |
| for SOF10 for subsequent studies | PK, activity, and immunogenicity endpoints |
| Exploratory Objectives | |
| To make a preliminary assessment of the | OS, defined as the time from the first SOF10 |
| activity of SOF10 when administered in | treatment day to death from any cause |
| combination with atezolizumab | |
| To evaluate potential relationships between | Relationship between plasma concentration or PK |
| drug exposure and the efficacy and safety of | parameters for SOF10 and safety endpoints |
| SOF10 | Relationship between plasma concentration or PK |
| | parameters for SOF10 and activity endpoints |
| To evaluate potential effects of ADAs | Relationship between ADA status and safety, PK, |
| | or activity endpoints |

ADA = anti-drug antibody; CR = complete response; CTCAE v5.0 = National Cancer Institute's Common Terminology Criteria for Adverse Events, Version 5.0; DOR = duration of response; ORR = objective response rate; OS = overall survival; PD = pharmacodynamic; PFS = progression-free survival; PK = pharmacokinetic; PR = partial response; RECIST v1.1 = Response Evaluation Criteria in Solid Tumors, Version 1.1.

### B. Study Design

i. Description of the Substudy

This urothelial carcinoma substudy is designed to evaluate the safety, tolerability, pharmacokinetics, immunogenicity, and preliminary anti-tumor activity of SOF10 when administered in combination with atezolizumab in patients with locally advanced or metastatic UC.

The substudy includes a screening period of up to 28 days, a treatment period, a minimum follow-up period of 90 days after treatment, and survival follow-up.

Approximately 40 patients with locally advanced or metastatic UC are to be enrolled in this substudy at approximately 20 global investigative sites. The substudy includes two stages: an initial safety run-in stage and an expansion stage. Patients in the safety run-in receive at least one cycle of study treatment, with both SOF10 and atezolizumab given concurrently starting from Cycle 1. Accrual in the expansion stage begins after the safety run-in stage has fully enrolled.

Patients in this substudy are initially assessed for eligibility during the screening period (lasting ≤28 days). Following confirmation of eligibility, patients receive SOF10 in combination with atezolizumab. Patients receive study treatment in 21-day cycles. Treatment is continued as long as patients are experiencing clinical benefit in the opinion of the investigator (e.g., in the absence of unacceptable toxicity or symptomatic deterioration attributed to disease progression) after an integrated investigator assessment of radiographic data, tissue biopsy results (if available), and clinical status. Patients are permitted to continue study treatment after disease progression per Response Evaluation Criteria in Solid Tumors, Version 1.1 (RECIST v1.1) if they meet certain criteria, including, but not limited to, evidence of clinical benefit as assessed by the investigator.

Patients undergo tumor assessments at screening (baseline) and at regular intervals during the study, which is measured by RECIST v1.1. All patients are closely monitored for adverse events throughout the study and for at least 90 days after the final dose of study treatment or until initiation of another systemic anti-cancer therapy, whichever occurs first. Adverse events are graded according to the National Cancer Institute's Common Terminology Criteria for Adverse Events, Version 5.0 (CTCAE v5.0).

All patients in the study are followed for survival and subsequent anti-cancer therapy information approximately every 3 months until death, loss to follow-up, or until Sponsor decision to discontinue survival follow-up because no further clinical development of SOF10 is planned or the study is terminated by the Sponsor, unless the patient requests to be withdrawn from follow-up.

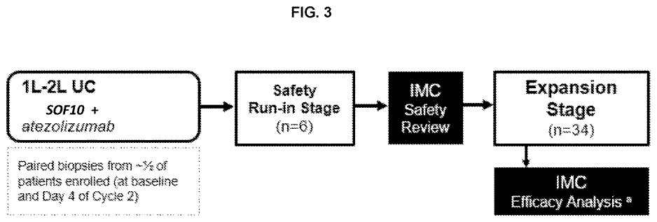

The study design is shown in FIG. 3.

### Safety Run-In Stage

Approximately 6 patients are to be enrolled in a safety run-in stage.

Dosing of the first 2 patients in the safety run-in stage is separated by at least 24 hours. After 6 patients have received at least one cycle of study treatment in the safety run-in stage, safety data is formally reviewed by the investigator.

### Expansion Stage

Accrual in the expansion stage begins after the safety run-in stage has been fully enrolled (e.g., 6 patients have received at least one cycle of study treatment). Approximately 34 additional patients are to be enrolled in the expansion stage for a total of approximately 40 patients enrolled in the substudy.

Patients receive study treatment as described herein to obtain additional safety, tolerability, and pharmacokinetic (PK) data, as well as preliminary evidence of clinical activity.

Serial Biopsies Up to approximately half of the patients enrolled in each cohort must have safely accessible tumor lesions available for serial biopsies, as described in Example 1.
Treatment after Disease Progression

Patients may continue study treatment in the safety run-in or expansion stages of the study as described in Example 1 and, e.g., FIG. 2.

### Duration of Participation

The total duration of study participation for each patient is expected to range from 1 day to more than 12 months.

### C. Materials and Methods

Approximately 40 patients with urothelial carcinoma are to be enrolled in this study.

ii. Patients

### Inclusion Criteria

Patients must meet the following criteria for study entry:
Age ≥18 years at time of signing Informed Consent Form
ECOG Performance Status of 0 or 1 (e.g., see Table 3)
   ECOG Performance Status of 2 is allowed for patients who are ineligible for cisplatin-containing chemotherapy.
Adequate hematologic and end-organ function, defined by the following laboratory test results, obtained within 14 days prior to initiation of study treatment:
   ANC≥1.0×10⁹/L (1000/μL) without granulocyte colony-stimulating factor support.
   Lymphocyte count ≥0.5×10⁹/L (≥500/μL).
   WBC count ≥2.5×10⁹/L (2500/μL).
   Platelet count ≥100×10⁹/L (≥100,000/μL) without transfusion.
   Hemoglobin ≥90 g/L (≥9 g/dL).
      Patients must not have been transfused within 2 weeks prior to screening to meet this criterion.
   AST, ALT, and ALP 2.5×ULN, with the following exceptions:
      Patients with documented liver metastases: AST and ALT 5×ULN.
      Patients with documented liver or bone metastases: ALP 5×ULN.
   Total bilirubin 1.5×ULN, with the following exception:
   Creatinine clearance calculated through use of the Cockcroft-Gault formula: ≥30 mL/min.
   Albumin 25 g/L (≥2.5 g/dL).
   For patients not receiving therapeutic anticoagulation: INR and aPTT 1.5×ULN.
      Patients receiving therapeutic anticoagulation should be on a stable dose with INR in target range.
Measurable disease (at least one target lesion) according to RECIST v1.1 on CT or MRI images within 28 days prior to enrollment.
   Previously irradiated lesions can be considered as measurable disease only if progressive disease has been unequivocally documented at that site since radiation.
   Lesions that are intended to be biopsied must not count as target lesions.
Histologically documented, locally advanced (T4b, any N; or any T, N2-N3), or metastatic UC (M1, Stage IV) (also termed transitional cell carcinoma or urothelial cell carcinoma of the urinary tract; including renal pelvis, ureters, urinary bladder, and urethra).
   Enrollment can be managed to ensure that approximately up to half of the accrued patients are positive for PD-L1 expression (e.g., ≥1%) as determined by a local laboratory through use of a health authority-approved or CE-marked assay.
Patients previously untreated for UC or treated for UC with at least one platinum-containing regimen are eligible.
   Previously untreated patients must not be eligible for any cisplatin-containing chemotherapy. Ineligible (“unfit”) for cisplatin-based chemotherapy is defined by any one of the following criteria:
      Impaired renal function (glomerular filtration rate (GFR) 230 mL/min but
         <60 mL/min); GFR should be assessed by direct measurement (e.g., creatinine clearance or ethyldediaminetetra-acetate) or, if not available, by calculation from serum/plasma creatinine.
      A hearing loss (measured by audiometry) of 25 dB at two contiguous frequencies.
      Grade 2 peripheral neuropathy (e.g., sensory alteration or paresthesias including tingling).
      ECOG Performance Status of 2.
   Previously treated patients must have had disease progression during or following treatment with at least one platinum-containing regimen (e.g., gemcitabine and cisplatin or carboplatin; methotrexate, vinblastine, doxorubicin, and cisplatin):
      A regimen is defined as patients receiving at least two cycles of a platinum-containing regimen.
      Patients who received prior adjuvant/neoadjuvant chemotherapy and progressed within 12 months of treatment with a platinum-containing adjuvant/neoadjuvant regimen can be considered as second-line patients.
      Patients who have received one cycle of a platinum-containing regimen but discontinued because of a Grade 4 hematologic toxicity or a Grade 3-4 non-hematologic toxicity may also be eligible.
      Patients may have received no more than two prior regimens of treatment (including the required platinum-based regimen) for their advanced or metastatic UC. Patients must have demonstrated disease progression during or following all prior regimen(s).

Patients with disease progression following chemoradiotherapy must demonstrate progression outside the prior radiotherapy port.

### Exclusion Criteria

Patients who meet any of the following criteria are excluded from study entry:
Prior treatment with a T-cell co-stimulating therapy or a CPI including anti-CTLA-4, anti-PD-1, and anti-PD-L1 therapeutic antibodies.
   Adjuvant or neoadjuvant treatment with cancer immunotherapy for localized disease is permitted if discontinued at least 6 months prior to Day 1 of Cycle 1.
Any anti-cancer therapy, whether investigational or approved, including chemotherapy, hormonal therapy, and/or radiotherapy, within 3 weeks prior to initiation of study treatment, with the following exceptions:
   Hormone-replacement therapy or oral contraceptives
   Herbal therapy intended for the treatment of cancer ≥7 days before Day 1 of Cycle 1
   Palliative radiotherapy for painful metastases or metastases in potentially sensitive locations (e.g., epidural space) ≥2 weeks prior to Day 1 of Cycle 1
Treatment with cancer vaccines within 6 weeks or 5 drug elimination half-lives (whichever is shorter) prior to initiation of study treatment.
Treatment with systemic immunostimulatory agents (including, but not limited to, IFNs and IL-2) within 4 weeks or 5 drug-elimination half-lives (whichever is longer) prior to initiation of study treatment and during study treatment.
Prior treatment with TGF-β or TGF-β receptor inhibitor (e.g., bintrafusp alpha, SRK-181, NIS793, and the like)
Symptomatic, untreated, or actively progressing CNS metastases.
   Asymptomatic patients with treated CNS lesions are eligible, provided that all of the following criteria are met:
      Measurable disease per RECIST v1.1 must be present outside the CNS.
      No evidence of acute or subacute brain metastasis-related hemorrhage was seen on CNS imaging at screening.
      The patient has not undergone stereotactic radiotherapy within 7 days prior to initiation of study treatment, whole-brain radiotherapy within 14 days prior to initiation of study treatment, or neurosurgical resection within 28 days prior to initiation of study treatment.
      The patient has no ongoing requirement for corticosteroids as therapy for CNS disease, with corticosteroids discontinued for ≥2 weeks prior to enrollment. Anticonvulsant therapy at a stable dose is permitted.
      There is no evidence of interim progression between completion of CNS-directed therapy and initiation of study treatment.
      Metastases are limited to the cerebellum or the supratentorial region (i.e., no metastases to the midbrain, pons, medulla, or spinal cord).
      Note: Asymptomatic patients with CNS metastases newly detected at screening are eligible for the study after receiving radiotherapy or surgery, with no need to repeat the screening brain scan.
Untreated keratoacanthoma.
   Patients who have undergone successful treatment for keratoacanthoma (i.e., surgical resection or other appropriate procedure) in the investigator's judgment prior to initiation of study treatment are eligible.
History of leptomeningeal disease.
Spinal cord compression not definitively treated with surgery and/or radiation, or previously diagnosed and treated spinal cord compression without evidence that disease has been clinically stable for ≥2 weeks prior to screening.
Significant cardiovascular disease (such as New York Heart Association Class II or greater cardiac disease, myocardial infarction, or cerebrovascular accident) within 3 months prior to initiation of study treatment, unstable arrhythmia, or unstable angina.
Any disease, metabolic dysfunction, physical examination finding, or clinical laboratory finding that contraindicates the use of an investigational drug, may affect the interpretation of the results, or may render the patient at high risk from treatment complications, including, but not limited to:
   Known clinically significant liver disease, including active viral, alcoholic, or other hepatitis, cirrhosis, and inherited liver disease or current alcohol abuse.
   Severe dyspnea or requirement for supplemental oxygen at rest.
   History of Stevens-Johnson syndrome, toxic epidermal necrolysis, or drug rash with eosinophilia and systemic symptoms.
Uncontrolled tumor-related pain.
   Patients requiring pain medication must be on a stable regimen at study entry.
   Symptomatic lesions (e.g., bone metastases or metastases causing nerve impingement) amenable to palliative radiotherapy should be treated prior to enrollment. Patients should be recovered from the effects of radiation.
   Asymptomatic metastatic lesions that would likely cause functional deficits or intractable pain with further growth (e.g., epidural metastasis that is not currently associated with spinal cord compression) should be considered for loco-regional therapy, if appropriate, prior to enrollment.
Uncontrolled pleural effusion, pericardial effusion, or ascites requiring recurrent drainage procedures (once monthly or more frequently).
   Patients with indwelling catheters (e.g., PLEURX®) are eligible.
Uncontrolled or symptomatic hypercalcemia (ionized calcium >1.5 mmol/L, calcium >12 mg/dL, or corrected calcium>ULN).
History of malignancy other than the disease under study within 3 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer.
Adverse events from prior anti-cancer therapy (with the exception of immune-related adverse events attributed to cancer immunotherapy; see below) that have not resolved to Grade 1 except for alopecia, vitiligo, or endocrinopathy managed with replacement therapy.
Any history of an immune-mediated Grade 4 adverse event attributed to prior cancer immunotherapy (other than endocrinopathy managed with replacement therapy or asymptomatic elevation of serum amylase and/or lipase).
Any history of an immune-mediated Grade 3 adverse event attributed to prior cancer immunotherapy (other than endocrinopathy managed with replacement therapy or asymptomatic elevation of serum amylase and/or lipase) that resulted in permanent discontinuation of the prior immunotherapeutic agent and/or occurred 6 months prior to planned Day 1 of Cycle 1
Any immune-mediated adverse event related to prior cancer immunotherapy (other than endocrinopathy managed with replacement therapy or stable vitiligo) that has not resolved completely to baseline
   Patients treated with corticosteroids for immune-mediated adverse events must demonstrate absence of related symptoms or signs for ≥4 weeks following discontinuation of corticosteroids.
Active or history of autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions:
   Patients with a history of autoimmune-mediated hypothyroidism who are on thyroid-replacement hormone are eligible for the study.
   Patients with controlled Type 1 diabetes mellitus who are on an insulin regimen are eligible for the study.
   Patients with eczema, psoriasis, lichen simplex chronicus, or vitiligo with dermatologic manifestations only (e.g., patients with psoriatic arthritis are excluded) are eligible for the study, provided all of following conditions are met:
      Rash must cover <10% of body surface area.
      Disease is well controlled at baseline and requires only low-potency topical corticosteroids.
      No occurrence of acute exacerbations of the underlying condition requiring psoralen plus ultraviolet A radiation, methotrexate, retinoids, biologic agents, oral calcineurin inhibitors, or high-potency or oral corticosteroids within the previous 12 months.
History of idiopathic pulmonary fibrosis, organizing pneumonia (e.g., bronchiolitis obliterans), drug-induced pneumonitis, or idiopathic pneumonitis, or evidence of active pneumonitis on screening chest CT scan.
   History of radiation pneumonitis in the radiation field (fibrosis) is permitted.
Active tuberculosis.
Severe infection within 4 weeks prior to initiation of study treatment, including, but not limited to, hospitalization for complications of infection, bacteremia, or severe pneumonia.
Treatment with IV antibiotics within 2 weeks prior to initiation of study treatment.
   Patients receiving prophylactic antibiotics (e.g., to prevent a urinary tract infection or chronic obstructive pulmonary disease (COPD) exacerbation) are eligible for the study.
Positive test for HIV infection.
Positive hepatitis B surface antigen (HbsAg) test, and/or positive total hepatitis B core antibody (HbcAb) test at screening.
   Patients with positive total HbcAb test followed by a negative hepatitis B virus (HBV) DNA test at screening can be enrolled.
   Antiviral prophylaxis for patients at risk for HBV reactivation is permitted.
Positive hepatitis C virus (HCV) antibody test at screening.
   Patients positive for HCV antibody are eligible only if PCR is negative for HCV RNA.
Known infection with SARS-CoV-2 (the virus that causes COVID-19), persistent symptoms of known prior SARS-CoV-2 infection, and/or known positive SARS-CoV-2 test within 4 weeks prior to screening.
Administration of a live, attenuated vaccine (e.g., FLUMIST®) within 4 weeks before first study treatment or anticipation that such a live, attenuated vaccine may be required during the study or within 5 months after the final dose of study treatment.
Treatment with systemic immunosuppressive medication (including, but not limited to, corticosteroids, cyclophosphamide, azathioprine, methotrexate, thalidomide, and anti-tumor necrosis factor (TNF) agents) within 2 weeks prior to initiation of study treatment, or anticipation of need for systemic immunosuppressive medication during study treatment, with the following exceptions:
   Patients who received acute, low-dose systemic immunosuppressant medication or a one-time pulse dose of systemic immunosuppressant medication (e.g., 48 hours of corticosteroids for a contrast allergy) are eligible for the study.
   Patients who received mineralocorticoids (e.g., fludrocortisone), inhaled or low-dose corticosteroids for COPD or asthma, or low-dose corticosteroids for orthostatic hypotension or adrenal insufficiency are eligible for the study.
Major surgical procedure or significant traumatic injury within 28 days prior to first study treatment, or anticipation of the need for major surgery before the end of the treatment period
   After major surgery, the patient must wait until surgical wounds are fully healed before initiating treatment.
Prior allogeneic stem cell or organ transplantation.
History of severe allergic anaphylactic reactions to chimeric or humanized antibodies or fusion proteins.
Known allergy or hypersensitivity to Chinese hamster ovary cell products, any component of the SOF10 formulation, or any of the study drugs or their excipients.
Known hypersensitivity to Chinese hamster ovary cell products or to any component of the atezolizumab formulation.

### Study Treatment and Other Treatments Relevant to the Study Design

### Study Treatment Formulation and Packaging

The investigational medicinal products for this study are SOF10 and atezolizumab.

### SOF10

The SOF10 drug product is supplied as a sterile liquid in a single-use, 10-ml glass vial. The vial contains approximately 10 mL (600 mg) of SOF10 solution.

### Atezolizumab

The atezolizumab drug product is supplied as a sterile liquid in a single-use, 20-mL glass vial. The vial contains approximately 20 mL (1200 mg) of atezolizumab solution. Atezolizumab is administered by IV infusion at a fixed dose of 1200 mg on Day 1 of each 21-day cycle in combination with SOF10. Atezolizumab is administered after SOF10 and the subsequent observation period, as described in Example 1. Atezolizumab infusions is administered per the instructions outlined in Table 4 of Example 1 including for patients who are administered atezolizumab alone (e.g., due to adverse events associated with SOF10).

iii. Study Treatment Dosage, Administration, and Compliance

An integrated approach based on the totality of nonclinical data, clinical pharmacology data, and preliminary Phase I clinical data from an ongoing phase I study may be used to select the initial SOF10 dose for this study, which may be no higher than the highest dose cleared in the ongoing phase I study, which is expected to be 1800 mg Q3W. SOF10 is administered by IV infusion at a fixed dose of 1800 mg on Day 1 of each 21-day cycle in combination with atezolizumab. The dose level of SOF10 will be based on the totality of data from the ongoing phase I study, and may be updated once additional data from the ongoing phase I study becomes available. At no time will the dose of SOF10 exceed the maximum administered dose (MAD) or MTD determined in the ongoing phase I study.

The initial dose of SOF10 is delivered over 60 (±10) minutes (although the infusion may be slowed or interrupted for patients who experience infusion-associated symptoms), followed by a 60-minute observation period prior to dosing with atezolizumab. If the 60-minute infusion is tolerated without infusion-associated adverse events, the second infusion may be delivered over 30 (±10) minutes, followed by a 30-minute observation period (e.g., see Table 6 of Example 1).

Guidance on study drug administration in the context of management of specific adverse events is provided in Table 5 of Example 1.

### Atezolizumab

Atezolizumab is administered by IV infusion at a fixed dose of 1200 mg on Day 1 of each 21-day cycle in combination with SOF10. Atezolizumab is administered after SOF10 and the subsequent observation period. Instructions for vital sign monitoring are provided Table 7 of Example 1. Atezolizumab infusions are administered per the instructions outlined in Table 7 of Example 1, including for patients who are administered atezolizumab alone (i.e., due to adverse events associated with SOF10).

Administration of atezolizumab is performed in a monitored setting where there is immediate access to trained personnel and adequate equipment and medicine to manage potentially serious reactions.

### Tumor and Response Evaluations

Patients undergo tumor assessments at baseline and following initiation of study treatment, regardless of dose delays, until radiographic disease progression according to RECIST v1.1. Thus, tumor assessments are to continue according to schedule in patients who discontinue treatment for reasons other than disease progression or loss of clinical benefit. In one example, tumor assessment is performed every 9 weeks (±1 week) for the first 54 weeks and then every 12 weeks (±2 weeks) thereafter. At the investigator's discretion, tumor assessments may be repeated at any time if progressive disease is suspected.

### Example 3. Phase I Study of SOF10 Plus Atezolizumab in Patients with Advanced/Recurrent Solid Tumours

A Phase I open-label, multicenter dose-escalation study was designed to evaluate the safety, pharmacokinetics (PK), and anti-tumour activity of the anti-latent TGF-beta 1 (TGF-β1) antibody SOF10 when administered in combination with atezolizumab in cancer patients having advanced or recurrent solid tumours. Patient cancer types in this study include gastrointestinal stromal tumor (GIST), skin cancer, colorectal cancer, ovarian (OV) cancer, renal cancer, and gallbladder cancer.

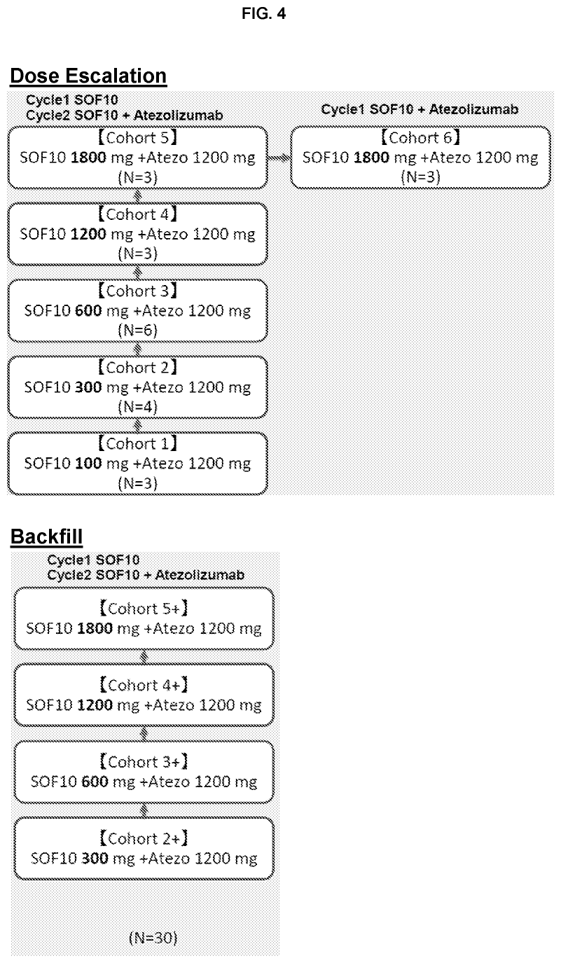

Six patient cohorts are described. Patients in Cohorts 1-5 in the dose escalation phase of this study were intravenously (IV) administered SOF10 during a first cycle (Cycle 1), followed by an IV administration of SOF10 (e.g., 100-1800 mg) in combination with atezolizumab (1200 mg) in a second cycle (Cycle 2) (FIG. 4). Patients in Cohort 6 in the dose escalation phase of this study were IV administered SOF10 (1800 mg) in combination with atezolizumab (1200 mg) during Cycle 1 (FIG. 4). A backfill of patients were enrolled for Cohorts 2-5 (FIG. 4). A total of 52 patients were enrolled (e.g., 22 for the dose escalation phase and 30 in backfill). Patients in Cohort 1 received 100 mg of SOF10 and 1200 mg of atezolizumab during Cycle 2 (n=3). Patients in Cohort 2 received 300 mg of SOF10 and 1200 mg of atezolizumab during Cycle 2 (n=4). Patients in Cohort 3 received 600 mg of SOF10 and 1200 mg of atezolizumab during Cycle 2 (n=6). Patients in Cohort 4 received 1200 mg of SOF10 and 1200 mg of atezolizumab during Cycle 2 (n=3). Patients in Cohort 5 received 1800 mg of SOF10 and 1200 mg of atezolizumab during Cycle 2 (n=3). Patients in Cohort 6 received 1800 mg of SOF10 and 1200 mg of atezolizumab during Cycle 1 (n=3).

Combinatorial administrations of SOF10 and atezolizumab were performed every 3 weeks (Q3W) and tolerability was evaluated. Primary endpoints included safety and PK, while secondary endpoints included anti-tumour activity per the Response Evaluation Criteria in Solid Tumors, Version 1.1 (RECIST v1.1).

Adverse events (AE) were observed in 42 patients (80.7%). Treatment-related adverse events (TRAEs) were observed in 30 patients (57.7%). The most common TRAE was rash (grade 1-2) and was not dose dependent. One dose-limiting toxicity (DLT) (liver disorder) was observed in a patient from Cohort 6 (e.g., 1800 mg of SOF10 and 1200 mg of atezolizumab during Cycle 1). A maximum tolerated dose was not identified, but tolerability was confirmed for all six cohorts. A summary of the safety profile is shown in Table 10.

TABLE 10
Safety Profile Summary

| MedDRA | | Cohort 1 | Cohort 2 | Cohort 3 | Cohort 4 | Cohort 5 | Cohort 6 |
|---|---|---|---|---|---|---|---|
| System | | SOF10 | SOF10 | SOF10 | SOF10 | SOF10 | SOF10 |
| Organ Class | | (100 mg) + | (300 mg) + | (600 mg) + | (1200 mg) + | (1800 mg) + | (1800 mg) + |
| MedDRA | | atez. | atez. | atez. | atez. | atez. | atez. |
| preferred | | (1200 mg) | (1200 mg) | (1200 mg) | (1200 mg) | (1200 mg) | (1200 mg) |
| Term | Grade | n = 3 | n = 14 | n = 12 | n = 10 | n = 10 | n = 3 |
| Any AE | Any | 3 (100%) | 9 (64.3%) | 11 (91.7%) | 9 (90%) | 9 (90%) | 1 (33.3%) |
| | >3 | 0 | 1 (7.1%) | 3 (25%) | 1 (10%) | 2 (20%) | 0 |
| Related to | Any | 3 (100%) | 9 (64.3%) | 6 (50%) | 4 (40%) | 7 (70%) | 1 (33.3%) |
| study treatment | | | | | | | |
| | >3 | 0 | 1 (7.1%) | 1 (8.3%) | 0 | 1 (10%) | 0 |
| Related to | Any | 3 (100%) | 7 (50%) | 6 (50%) | 4 (40%) | 6 (60%) | 1 (33.3%) |
| SOF10 | | | | | | | |
| | >3 | 0 | 1 (7.1%) | 1 (8.3%) | 0 | 1 (10%) | 0 |
| Related to | Any | 2 (66.7%) | 7 (50%) | 5 (41.7%) | 3 (30%) | 5 (50%) | 0 |
| atez. | | | | | | | |
| | >3 | 0 | 0 | 1 (8.3%) | 0 | 0 | 0 |
| TRAE occurring | | | | | | | |
| in >3 patients | | | | | | | |
| Rash | Any | 3 (100%) | 3 (21.4%) | 3 (35%) | 1 (10%) | 3 (30%) | 0 |
| Pruritus | Any | 1 (33.3%) | 3 (21.4%) | 2 (16.7%) | 0 | 1 (10%) | 0 |
| Nausea | Any | 0 | 1 (7.1%) | 1 (8.3%) | 2 (20%) | 1 (10%) | 0 |

AE = adverse event;
TRAE = treatment-related adverse events;
atez. = atezolizumab

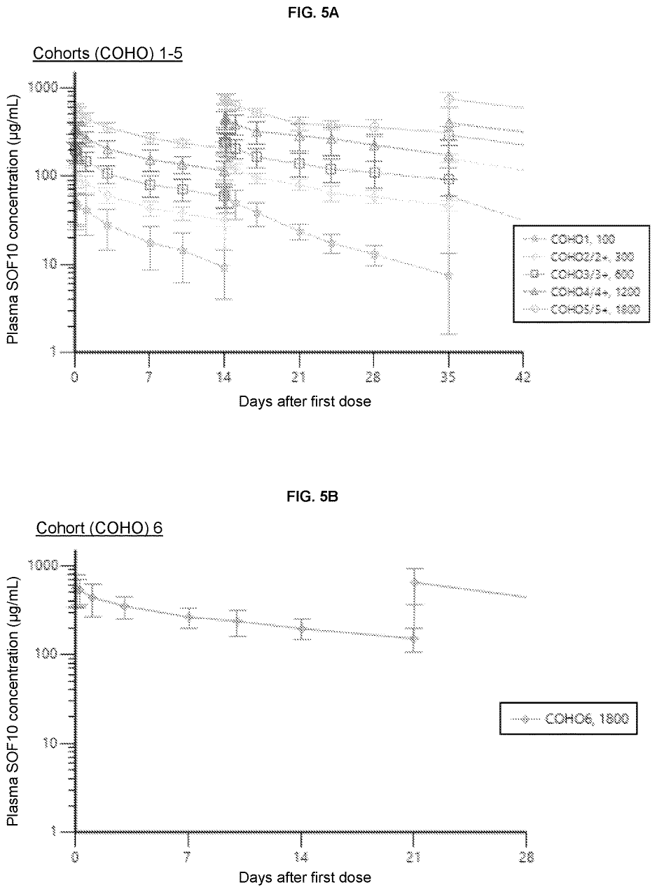

As shown in FIGS. 5A and 51B, SOF10 exposure levels increased with dosage; SOF10 PK profile at Cycle 1 was similar between Cohort 2 (FIG. 5A) and Cohort 6 (FIG. 5); and terminal slopes were smaller in cohorts that received ≥300 mg of SOF10.

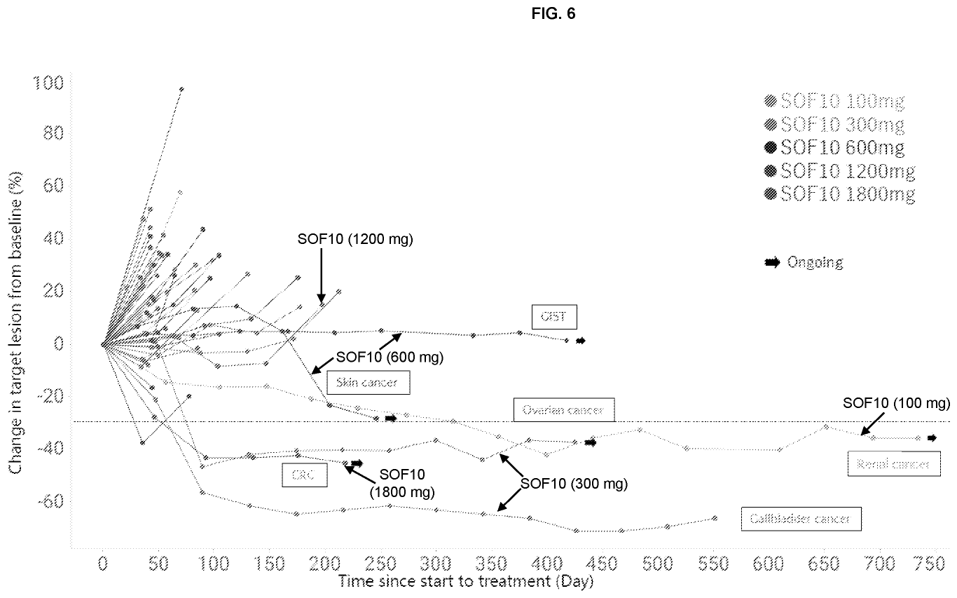

Anti-drug antibodies (ADAs) were seen in three patients (5.8%): one patient from Cohort 1, one patient from Cohort 2, and one patient from Cohort 4. Overall, 4 of the 52 patients experienced a partial response, resulting in a confirmed objective response rate (ORR) of 8% per RECIST v1.1. A summary of treatment response is shown in Table 11 and treatment efficacy (i.e., change in tumor size, relative to baseline, according to RECIST v1.1) in several cancers (e.g., GIST, skin cancer, colorectal cancer, OV cancer, renal cancer, and gallbladder cancer) is shown in FIG. 6.

TABLE 11
Summary of Responses

| | Cohort 1 | Cohort 2 | Cohort 3 | Cohort 4 | Cohort 5 | Cohort 6 |
|---|---|---|---|---|---|---|
| | SOF10 | SOF10 | SOF10 | SOF10 | SOF10 | SOF10 |
| | (100 mg) + | (300 mg) + | (600 mg) + | (1200 mg) + | (1800 mg) + | (1800 mg) + |
| | atez. | atez. | atez. | atez. | atez. | atez. |
| | (1200 mg) | (1200 mg) | (1200 mg) | (1200 mg) | (1200 mg) | (1200 mg) |
| | n = 3 | n = 14 | n = 12 | n = 10 | n = 10 | n = 3 |
| Responders | 1 (33.3%) | 2 (14.3%) | 0 | 0 | 1 (12.5%) | 0 |
| 95% CI for | 0.8, 90.6 | 1.8, 42.8 | 0, 26.5 | 0, 30.8 | 0.3, 52.7 | 0, 70.8 |
| response rates | | | | | | |
| Complete | 0 | 0 | 0 | 0 | 0 | 0 |
| response (CR) | | | | | | |
| 95% CI | 0, 70.8 | 0, 23.2 | 0, 26.5 | 0, 30.8 | 0, 36.9 | 0, 70.8 |
| Partial | 1 (33.3%) | 2 (14.3%) | 0 | 0 | 1 (12.5%) | 0 |
| response (PR) | | | | | | |
| 95% CI | 0.8, 90.6 | 1.8, 42.8 | 0, 26.5 | 0, 30.8 | 0.3, 52.7 | 0, 70.8 |
| Stable disease | 0 | 3 (21.4%) | 2 (16.7%) | 1 (10%) | 3 (37.5%) | 0 |
| (SD) | | | | | | |
| 95% CI | 0, 70.8 | 4.7, 50.8 | 2.1, 48.4 | 0.3, 44.5 | 8.5, 75.5 | 0, 70.8 |
| Progressive | 2 (66.7%) | 9 (64.3) | 10 (83.3%) | 9 (90%) | 4 (50%) | 3 (100%) |
| disease (PD) | | | | | | |
| 95% CI | 9.4, 99.2 | 35.1, 87.2 | 51.6, 97.9 | 55.5, 99.7 | 15.7, 84.3 | 29.2, 100 |
| Not evaluable | 0 | 0 | 0 | 0 | 0 | 0 |
| (NE) | | | | | | |
| Disease | 1 (33.3%) | 5 (35.7%) | 2 (16.7%) | 1 (10%) | 4 (50%) | 0 |
| control rate | | | | | | |
| 95% CI | 0.8, 90.6 | 12.8, 64.9 | 2.1, 48.4 | 0.3, 44.5 | 15.7, 84.3 | 0, 70.8 |

CI = confidence interval;
atez. = atezolizumab;
CR = complete response;
SD = stable disease;
PD = progressive disease;
NE = not evaluable

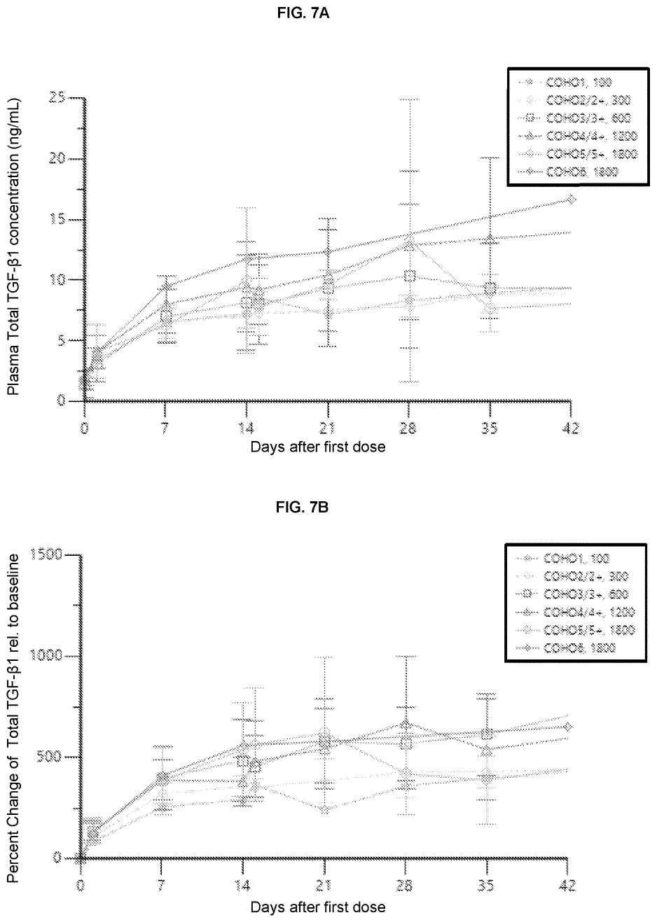

Following administration of the first dose of therapy, total TGF-β1 accumulated in the plasma of patients. There was no clear dose-dependent increase of TGF-β1 in either TGF-β1 total plasma concentrations (FIG. 7A) or relative TGF-β1 percent change to baseline TGF-β1 levels (FIG. 73).

In conclusion, administration of SOF10 in combination with atezolizumab was well tolerated in patients with advanced solid tumours as there were no new safety signals. SOF10 administration showed increases in exposure with dosages and the addition of atezolizumab did not appear to affect the PK of SOF10. The partial responses were durable. Select safety and response data described above are summarized in Table 12.

TABLE 12
Select Summary of Patient Safety and Response Data

| | Dose | | | | | Dose |
|---|---|---|---|---|---|---|
| | escalation | Dose escalation + backfill | | | | escalation |
| | 1 | 2 | 3 | 4 | 5 | 6 |
| Cohort ^{a} | (n = 3) | (n = 14) | (n = 12) | (n = 10) | (n = 10) | (n = 3) |
| SOF10 dose, mg | 100 | 300 | 600 | 1200 | 1800 | 1800 |
| Atezolizumab | 1200 | 1200 | 1200 | 1200 | 1200 | 1200 |
| dose, mg | | | | | | |

Safety, n (%)

| | | | | | | |
|---|---|---|---|---|---|---|
| TRAE | 3 (100%) | 9 (64.3%) | 6 (50%) | 4 (40%) | 7 (70%) | 1 (33.3%) |
| Grade 3-4 | 0 | 1 (7) | 1 (8) | 0 | 1 (10%) | 0 |

Confirmed best response, n (%)

| | | | | | | |
|---|---|---|---|---|---|---|
| Responders | 1 (33.3%) | 2 (14.3%) | 0 | 0 | 1 (12.5%) ^{b} | 0 |
| Partial | 1 (33.3%) | 2 (14.3%) | 0 | 0 | 1 (12.5%) ^{b} | 0 |
| response^{c} | | | | | | |
| Disease | 1 (33.3%) | 5 (35.7%) | 2 (16.7%) | 1 (10%) | 4 (50%) ^{b} | 0 |
| control rate | | | | | | |

DE, dose escalation; TRAE, treatment-related adverse event.
^{a} Patients had skin, colorectal, ovarian, gallbladder, renal or gastrointestinal stromal cancers.
^{b} Cohort 5 efficacy analysis: n = 8.
^{c}In patients with renal, gallbladder, ovarian and colorectal cancer, respectively.

### SEQUENCE LISTING

Table 13 provides a listing of sequences disclosed herein.

TABLE 13
Sequence Listing

| SEQ | |
|---|---|
| ID | |
| NO | Sequence |
| 1 | SEAMN |
| 2 | YIYTSGTTYRANWARG |
| 3 | GTGIYDYYYWVMDL |
| 4 | QASQSISTYLA |
| 5 | AASTLES |
| 6 | QSYSDGDSVG |
| 7 | QVQLVESGGGVVQPGRSLRLSCAASGFTFSSEAMNWIRQPPGKGLEWIGYIYTSGTTYRAN |
| | WARGRFTISRDNSKNTLYLQMNSLRAEDTAVYYCARGTGIYDYYYWVMDLWGPGTLVTVSS |
| 8 | DIQMTQSPSSLSASVGDRVTITCQASQSISTYLAWYQQKPGQPPKLLIYAASTLESGVPSRFSG |
| | SGSGTDFTLTISSLQPEDFATYYCQSYSDGDSVGFGQGTKVEIK |
| 9 | ASTKGPSVFPLAPSSKSTSGGTAALGCLVKDYFPEPVTVSWNSGALTSGVHTFPAVLQSSGLY |
| | SLSSVVTVPSSSLGTQTYICNVNHKPSNTKVDKRVEPKSCDKTHTCPPCPAPELRRGPSVFLF |
| | PPKPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLTCL |
| | VKGFYPSDIAVEWESNGQPENNYKTTPPVLDSDGSFFLYSKLTVDKSRWQQGNVFSCSVLHE |
| | ALHAHYTRKELSLSP |
| 10 | RTVAAPSVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC |
| 11 | QVQLVESGGGVVQPGRSLRLSCAASGFTFSSEAMNWIRQPPGKGLEWIGYIYTSGTTYRA |
| | NWARGRFTISRDNSKNTLYLQMNSLRAEDTAVYYCARGTGIYDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVLHEALHAHYTRKELSLSP |
| 12 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDGDSVGFGQGTKVEIKRTVAAPSVFIFP |
| | PSDEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |
| 13 | SYAMN |
| 14 | YIYTSGTTYHANWARG |
| 15 | GTFIWDYYYWVMDL |
| 16 | QASQSISTYLA |
| 17 | KASTLES |
| 18 | QSYSDADSVG |
| 19 | SYAMN |
| 20 | YIYTSGTRYRANWARG |
| 21 | GTDIYDYYYWVMDL |
| 22 | QASQSISTYLA |
| 23 | AASTLES |
| 24 | QSYSDGDSVG |
| 25 | SYAMN |
| 26 | YIYTSGTRYRANWARG |
| 27 | GTDIYDYYYWVMDL |
| 28 | QASQSISTYLA |
| 29 | AASTLES |
| 30 | QSYSDADSVG |
| 31 | QVQLVESGGGVVQPGRSLRLSCAASGFTFSSYAMNWIRQPPGKGLEWIGYIYTSGTTYHA |
| | NWARGRFTISRDNSKNTLYLQMNSLRAEDTAVYYCARGTFIWDYYYWVMDLWGPGTLVTV |
| | SS |
| 32 | DIQMTQSPSSLSASVGDRVTITCQASQSISTYLAWYQQKPGQPPKLLIYKASTLESGVPS |
| | RFSGSGSGTDFTLTISSLQPEDFATYYCQSYSDADSVGFGQGTKVEIK |
| 33 | QVQLVESGGGVVQPGRSLRLSCAASGFTFSSYAMNWIRQPPGKGLEWIGYIYTSGTRYRA |
| | NWARGRFTISRDNSKNTLYLQMNSLRAEDTAVYYCARGTDIYDYYYWVMDLWGPGTLVTV |
| | SS |
| 34 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDGDSVGFGQGTKVEIK |
| 35 | QVQLVESGGGVVQPGRSLRLSCAASGFTFSSYAMNWIRQPPGKGLEWIGYIYTSGTRYRA |
| | NWARGRFTISRDNSKNTLYLQMNSLRAEDTAVYYCARGTDIYDYYYWVMDLWGPGTLVTV |
| | SS |
| 36 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDADSVGFGQGTKVEIK |
| 37 | QVQLVESGGGVVQPGRSLRLSCAASGFTFSSYAMNWIRQPPGKGLEWIGYIYTSGTTYHA |
| | NWARGRFTISRDNSKNTLYLQMNSLRAEDTAVYYCARGTFIWDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSP |
| 38 | DIQMTQSPSSLSASVGDRVTITCQASQSISTYLAWYQQKPGQPPKLLIYKASTLESGVPS |
| | RFSGSGSGTDFTLTISSLQPEDFATYYCQSYSDADSVGFGQGTKVEIKRTVAAPSVFIFP |
| | PSDEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |
| 39 | QVQLVESGGGVVQPGRSLRLSCAASGFTFSSEAMNWIRQPPGKGLEWIGYIYTSGTTYRA |
| | NWARGRFTISRDNSKNTLYLQMNSLRAEDTAVYYCARGTGIYDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSP |
| 40 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDGDSVGFGQGTKVEIKRTVAAPSVFIFP |
| | PSDEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |
| 41 | QVQLVESGGGVVQPGRSLRLSCAASGFTFSSYAMNWIRQPPGKGLEWIGYIYTSGTRYRA |
| | NWARGRFTISRDNSKNTLYLQMNSLRAEDTAVYYCARGTDIYDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSP |
| 42 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDGDSVGFGQGTKVEIKRTVAAPSVFIFP |
| | PSDEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |
| 43 | QVQLVESGGGVVQPGRSLRLSCAASGFTFSSYAMNWIRQPPGKGLEWIGYIYTSGTRYRA |
| | NWARGRFTISRDNSKNTLYLQMNSLRAEDTAVYYCARGTDIYDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSP |
| 44 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDADSVGFGQGTKVEIKRTVAAPSVFIFP |
| | PSDEQLKSGTASVVCLLNNFYPREAKVQWKVDNALQSGNSQESVTEQDSKDSTYSLSSTL |
| | TLSKADYEKHKVYACEVTHQGLSSPVTKSFNRGEC |
| 45 | QVQLVESGGGVVQPGRSLRLSCAASGFTFSSYAMNWIRQPPGKGLEWIGYIYTSGTTYHA |
| | NWARGRFTISRDNSKNTLYLQMNSLRAEDTAVYYCARGTFIWDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVLHEALHAHYTRKELSLSP |
| 46 | DIQMTQSPSSLSASVGDRVTITCQASQSISTYLAWYQQKPGQPPKLLIYKASTLESGVPS |
| | RFSGSGSGTDFTLTISSLQPEDFATYYCQSYSDADSVGFGQGTKVEIKRADAAPTVSIFP |
| | PSSEQLTSGGASVVCFLNNFYPKDINVKWKIDGSERQNGVLNSWTDQDSKDSTYSMSSTL |
| | TLTKDEYERHNSYTCEATHKTSTSPIVKSFNRNEC |
| 47 | QVQLVESGGGVVQPGRSLRLSCAASGFTFSSEAMNWIRQPPGKGLEWIGYIYTSGTTYRA |
| | NWARGRFTISRDNSKNTLYLQMNSLRAEDTAVYYCARGTGIYDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVLHEALHAHYTRKELSLSP |
| 48 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDGDSVGFGQGTKVEIKRADAAPTVSIFP |
| | PSSEQLTSGGASVVCFLNNFYPKDINVKWKIDGSERQNGVLNSWTDQDSKDSTYSMSSTL |
| | TLTKDEYERHNSYTCEATHKTSTSPIVKSFNRNEC |
| 49 | QVQLVESGGGVVQPGRSLRLSCAASGFTFSSYAMNWIRQPPGKGLEWIGYIYTSGTRYRA |
| | NWARGRFTISRDNSKNTLYLQMNSLRAEDTAVYYCARGTDIYDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVLHEALHAHYTRKELSLSP |
| 50 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDGDSVGFGQGTKVEIKRADAAPTVSIFP |
| | PSSEQLTSGGASVVCFLNNFYPKDINVKWKIDGSERQNGVLNSWTDQDSKDSTYSMSSTL |
| | TLTKDEYERHNSYTCEATHKTSTSPIVKSFNRNEC |
| 51 | QVQLVESGGGVVQPGRSLRLSCAASGFTFSSYAMNWIRQPPGKGLEWIGYIYTSGTRYRA |
| | NWARGRFTISRDNSKNTLYLQMNSLRAEDTAVYYCARGTDIYDYYYWVMDLWGPGTLVTV |
| | SSASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELR |
| | RGPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVLHEALHAHYTRKELSLSP |
| 52 | DIQMTQSPSSLSASVGDRVTITCQASQSISTYLAWYQQKPGQPPKLLIYAASTLESGVPS |
| | RFSGSGSGTDFTLTISSLQPEDFATYYCQSYSDADSVGFGQGTKVEIKRADAAPTVSIFP |
| | PSSEQLTSGGASVVCFLNNFYPKDINVKWKIDGSERQNGVLNSWTDQDSKDSTYSMSSTL |
| | TLTKDEYERHNSYTCEATHKTSTSPIVKSFNRNEC |
| 53 | MPPSGLRLLPLLLPLLWLLVLTPGRPAAGLSTCKTIDMELVKRKRIEAIRGQILSKLRLA |
| | SPPSQGEVPPGPLPEAVLALYNSTRDRVAGESAEPEPEPEADYYAKEVTRVLMVETHNEI |
| | YDKFKQSTHSIYMFFNTSELREAVPEPVLLSRAELRLLRLKLKVEQHVELYQKYSNNSWR |
| | YLSNRLLAPSDSPEWLSFDVTGVVRQWLSRGGEIEGFRLSAHCSCDSRDNTLQVDINGFT |
| | TGRRGDLATIHGMNRPFLLLMATPLERAQHLQSSRHRRALDTNYCFSSTEKNCCVRQLYI |
| | DFRKDLGWKWIHEPKGYHANFCLGPCPYIWSLDTQYSKVLALYNQHNPGASAAPCCVPQA |
| | LEPLPIVYYVGRKPKVEQLSNMIVRSCKCS |
| 54 | atgccgccct ccgggctgcg gctgctgccg ctgctgctac cgctgctgtg gctactggtg ctgacgcctg gccggccggc |
| | cgcgggacta tccacctgca agactatcga catggagctg gtgaagcgga agcgcatcga ggccatccgc ggccagatcc |
| | tgtccaagct gcggctcgcc agccccccga gccaggggga ggtgccgccc ggcccgctgc ccgaggccgt gctcgccctg |
| | tacaacagca cccgcgaccg ggtggccggg gagagtgcag aaccggagcc cgagcctgag gccgactact |
| | acgccaagga ggtcacccgc gtgctaatgg tggaaaccca caacgaaatc tatgacaagt tcaagcagag |
| | tacacacagc atatatatgt tcttcaacac atcagagctc cgagaagcgg tacctgaacc cgtgttgctc tcccgggcag |
| | agctgcgtct gctgaggctc aagttaaaag tggagcagca cgtggagctg taccagaaat acagcaacaa ttcctggcga |
| | tacctcagca accggctgct ggcacccagc gactcgccag agtggttatc ttttgatgtc accggagttg tgcggcagtg |
| | gttgagccgt ggaggggaaa ttgagggctt tcgccttagc gcccactgct cctgtgacag cagggataac acactgcaag |
| | tggacatcaa cgggttcact accggccgcc gaggtgacct ggccaccatt catggcatga accggccttt cctgcttctc |
| | atggccaccc cgctggagag ggcccagcat ctgcaaagct cccggcaccg ccgagccctg gacaccaact attgcttcag |
| | ctccacggag aagaactgct gcgtgcggca gctgtacatt gacttccgca aggacctcgg ctggaagtgg atccacgagc |
| | ccaagggcta ccatgccaac ttctgcctcg ggccctgccc ctacatttgg agcctggaca cgcagtacag caaggtcctg |
| | gccctgtaca accagcataa cccgggcgcc tcggcggcgc cgtgctgcgt gccgcaggcg ctggagccgc tgcccatcgt |
| | gtactacgtg ggccgcaagc ccaaggtgga gcagctgtcc aacatgatcg tgcgctcctg caagtgcagc tga |
| 55 | MPPSGLRLLPLLLPLPWLLVLTPGRPAAGLSTCKTIDMELVKRKRIEAIRGQILSKLRLA |
| | SPPSQGEVPPGPLPEAVLALYNSTRDRVAGESADPEPEPEADYYAKEVTRVLMVDRNNAI |
| | YEKTKDISHSIYMFFNTSDIREAVPEPPLLSRAELRLQRLKSSVEQHVELYQKYSNNSWR |
| | YLGNRLLTPTDTPEWLSFDVTGVVRQWLNQGDGIQGFRFSAHCSCDSKDNKLHVEINGIS |
| | PKRRGDLGTIHDMNRPFLLLMATPLERAQHLHSSRHRRALDTNYCFSSTEKNCCVRQLYI |
| | DFRKDLGWKWIHEPKGYHANFCLGPCPYIWSLDTQYSKVLALYNQHNPGASASPCCVPQA |
| | LEPLPIVYYVGRKPKVEQLSNMIVRSCKCS |
| 56 | atgccgccct cggggctgcg gctactgccg cttctgctcc cactcccgtg gcttctagtg ctgacgcccg ggaggccagc |
| | cgcgggactc tccacctgca agaccatcga catggagctg gtgaaacgga agcgcatcga agccatccgt ggccagatcc |
| | tgtccaaact aaggctcgcc agtcccccaa gccaggggga ggtaccgccc ggcccgctgc ccgaggcggt gctcgctttg |
| | tacaacagca cccgcgaccg ggtggcaggc gagagcgccg acccagagcc ggagcccgaa gcggactact |
| | atgctaaaga ggtcacccgc gtgctaatgg tggaccgcaa caacgccatc tatgagaaaa ccaaagacat ctcacacagt |
| | atatatatgt tcttcaatac gtcagacatt cgggaagcag tgcccgaacc cccattgctg tcccgtgcag agctgcgctt |
| | gcagagatta aaatcaagtg tggagcaaca tgtggaactc taccagaaat atagcaacaa ttcctggcgt taccttggta |
| | accggctgct gacccccact gatacgcctg agtggctgtc ttttgacgtc actggagttg tacggcagtg gctgaaccaa |
| | ggagacggaa tacagggctt tcgattcagc gctcactgct cttgtgacag caaagataac aaactccacg tggaaatcaa |
| | cgggatcagc cccaaacgtc ggggcgacct gggcaccatc catgacatga accggccctt cctgctcctc atggccaccc |
| | ccctggaaag ggcccagcac ctgcacagct cacggcaccg gagagccctg gataccaact attgcttcag ctccacagag |
| | aagaactgct gtgtgcggca gctgtacatt gactttagga aggacctggg ttggaagtgg atccacgagc ccaagggcta |
| | ccatgccaac ttctgtctgg gaccctgccc ctatatttgg agcctggaca cacagtacag caaggtcctt gccctctaca |
| | accaacacaa cccgggcgct tcggcgtcac cgtgctgcgt gccgcaggct ttggagccac tgcccatcgt ctactacgtg |
| | ggtcgcaagc ccaaggtgga gcagttgtcc aacatgattg tgcgctcctg caagtgcagc tga |
| 57 | MPPSGLRLLPLLLPLLWLLVLTPGRPAAGLSTCKTIDMELVKRKRIEAIRGQILSKLRLA |
| | SPPSQGEVPPGPLPEAVLALYNSTRDRVAGESAEPEPEPEADYYAKEVTRVLMVETHNEI |
| | YDKFKQSTHSIYMFFNTSELREAVPEPVLLSRAELRLLRLKLKVEQHVELYQKYSNNSWR |
| | YLSNRLLAPSDSPEWLSFDVTGVVRQWLSRGGEIEGFRLSAHCSCDSKDNTLQVDINGFT |
| | TGRRGDLATIHGMNRPFLLLMATPLERAQHLQSSRHRRALDTNYCFSSTEKNCCVRQLYI |
| | DFRKDLGWKWIHEPKGYHANFCLGPCPYIWSLDTQYSKVLALYNQHNPGASAAPCCVPQA |
| | LEPLPIVYYVGRKPKVEQLSNMIVRSCKCS |
| 58 | atgccgccct ccgggctgcg gctgctgccg ctgctgctac cgctgctgtg gctactggtg ctgacgcctg gccggccggc |
| | cgccggacta tccacctgca agactatcga catggagctg gtgaagcgga agcgcatcga ggccatccgc ggccagatcc |
| | tgtccaagct gcggctcgcc agccccccga gccaggggga ggtgccgccc ggcccgctgc ccgaggccgt gctcgccctg |
| | tacaacagca cccgcgaccg ggtggccggg gagagtgcgg agccggaacc cgaaccggag gccgactact |
| | acgccaagga ggtcacccgc gtgctaatgg tggaaaccca caacgaaatc tatgacaagt tcaagcagag |
| | cacacacagc atatatatgt tcttcaacac atcagagctc cgagaagcag tacctgaacc tgtgttgctc tcccgggcag |
| | agctgcgtct gctgaggctc aagttaaaag tggagcagca tgtggagctg taccagaaat acagcaacaa ttcctggcga |
| | tacctcagca accggctgct ggcgcccagc gactcgccgg agtggttgtc ttttgatgtc accggagttg tgcggcagtg |
| | gttgagccgc ggaggggaaa ttgagggctt tcgccttagc gcccactgct cctgtgacag caaagataac acactgcaag |
| | tggacatcaa cgggttcact accggccgcc gaggtgacct ggccaccatt catggcatga accggccttt cctgcttctc |
| | atggccaccc cgctggagag ggcccaacat ctgcaaagct cccggcaccg ccgagccctg gacaccaact actgcttcag |
| | ctccacggag aagaactgct gcgtgcggca gctgtatatt gacttccgca aggacctcgg ctggaagtgg atccacgagc |
| | ccaagggcta ccatgccaac ttctgcctgg gaccctgccc ctacatttgg agcctggaca cgcagtacag caaggtcctg |
| | gccctgtaca accagcataa cccgggcgcc tcggcggcgc cgtgctgcgt gccgcaggcg ctggagccgc tgcccatcgt |
| | gtactacgtg ggccgcaagc ccaaggtgga gcagctgtcc aacatgatcg tgcgctcctg caaatgcagc tga |

### OTHER EMBODIMENTS

Although the foregoing invention has been described in some detail by way of illustration and example for purposes of clarity of understanding, the descriptions and examples should not be construed as limiting the scope of the invention.

What is claimed is:

1. A method of treating a subject having a locally advanced, recurrent, or metastatic solid tumor, the method comprising administering to the subject an anti-cancer therapy comprising an anti-latent transforming growth factor (TGF)-beta 1 antibody at a dosage of 1800 mg, wherein the anti-latent TGF-beta 1 antibody comprises the following six hypervariable regions (HVRs):
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

2. The method of claim 1, wherein the anti-latent TGF-beta 1 antibody is administered to the subject in a dosing regimen comprising one or more 21-day dosing cycles.

3. The method of claim 2, wherein the anti-latent TGF-beta 1 antibody is administered to the subject on Day 1 of each 21-day dosing cycle.

4. A method of treating a subject having a locally advanced, recurrent, or metastatic solid tumor, the method comprising administering to the subject an anti-cancer therapy comprising an anti-latent TGF-beta 1 antibody at a dosage of 1800 mg every three weeks (Q3W), wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

5. A method of treating a subject having a locally advanced, recurrent, or metastatic solid tumor, the method comprising administering to the subject an anti-cancer therapy comprising an anti-latent TGF-beta 1 antibody at a dosage of 1200 mg, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

6. The method of claim 5, wherein the anti-latent TGF-beta 1 antibody is administered to the subject in a dosing regimen comprising one or more 28-day dosing cycles.

7. The method of claim 6, wherein the anti-latent TGF-beta 1 antibody is administered to the subject on Days 1 and 15 of each 28-day dosing cycle.

8. A method of treating a subject having a locally advanced, recurrent, or metastatic solid tumor, the method comprising administering to the subject an anti-cancer therapy comprising an anti-latent TGF-beta 1 antibody at a dosage of 1200 mg every two weeks (Q2W), wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

9. The method of any one of claims 1-8, wherein the anti-latent TGF-beta 1 antibody is administered to the subject intravenously.

10. The method of claim 9, wherein the anti-latent TGF-beta 1 antibody is administered to the subject intravenously by infusion.

11. The method of any one of claims 1-10, wherein a tumor sample from the subject has been determined to have a detectable level of PD-L1 expression.

12. The method of any one of claims 1-11, wherein the subject's age is 18 years or older.

13. The method of any one of claims 1-12, wherein the anti-cancer therapy is a first-line therapy.

14. The method of any one of claims 1-12, wherein the anti-cancer therapy is a second-line therapy or a third-line therapy.

15. The method of any one of claims 1-14, wherein the subject has not been previously treated with a checkpoint inhibitor.

16. The method of any one of claims 1-15, wherein the solid tumor is metastatic.

17. The method of any one of claims 1-16, wherein the locally advanced, recurrent, or metastatic solid tumor is non-small cell lung cancer (NSCLC), gastric cancer, pancreatic ductal adenocarcinoma (PDAC), urothelial carcinoma (UC), gastrointestinal stromal tumor (GIST), skin cancer, colorectal cancer, ovarian (OV) cancer, renal cancer, or gallbladder cancer.

18. The method of claim 17, wherein the locally advanced, recurrent, or metastatic solid tumor is NSCLC.

19. The method of claim 18, wherein the NSCLC is histologically or cytologically confirmed metastatic nonsquamous NSCLC or metastatic squamous NSCLC.

20. The method of claim 18 or 19, wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

21. The method of claim 20, wherein the subject has previously received a combination therapy comprising a platinum-containing chemotherapy regimen and a PD-1 axis binding antagonist.

22. The method of claim 20, wherein the subject has previously received a platinum-containing chemotherapy regimen and a PD-1 axis binding antagonist as individual regimens.

23. The method of any one of claims 20-22, wherein the subject had disease progression or recurrence within 6 months of definitive therapy for locally advanced NSCLC.

24. The method of any one of claims 18-23, wherein a tumor sample from the subject has been determined to have a detectable level of PD-L1 expression.

25. The method of claim 17, wherein the locally advanced, recurrent, or metastatic solid tumor is gastric cancer.

26. The method of claim 25, wherein the subject has unresectable locally advanced or metastatic gastric cancer that is histologically confirmed to be adenocarcinoma.

27. The method of claim 25 or 26, wherein the gastric cancer comprises esophagogastric junction cancer.

28. The method of any one of claims 25-27, wherein the gastric cancer is HER2-negative gastric cancer.

29. The method of any one of claims 25-28, wherein the subject is previously untreated for gastric cancer and/or the subject has not been previously treated with a checkpoint inhibitor.

30. The method of claim 17, wherein the locally advanced, recurrent, or metastatic solid tumor is PDAC.

31. The method of claim 30, wherein the subject has histologically or cytologically confirmed metastatic PDAC.

32. The method of claim 30 or 31, wherein the subject is previously untreated for the PDAC and/or the subject has not been previously treated with a checkpoint inhibitor.

33. The method of claim 17, wherein the locally advanced, recurrent, or metastatic solid tumor is UC.

34. The method of claim 33, wherein the subject has histologically documented, locally advanced (T4b, any N; or any T, N2-N3) UC, or metastatic UC (M1, Stage 4).

35. The method of claim 33 or 34, wherein the subject is previously untreated for UC.

36. The method of any one of claims 33-35, wherein the subject is ineligible for cisplatin-containing chemotherapy.

37. The method of claim 36, wherein the subject is ineligible for cisplatin-containing chemotherapy as defined by any one of the following criteria:
(i) impaired renal function in terms of glomerular filtration rate (GFR) of ≥30 mL/min but <60 mL/min as assessed by direct measurement or by calculation from serum or plasma creatinine;
(ii) hearing loss of 25 dB at two contiguous frequencies as measured by audiometry;
(iii) Grade 2 peripheral neuropathy; or
(iv) Eastern Cooperative Oncology Group (ECOG) Performance Status of 2.

38. The method of any one of claims 33-35, wherein the subject has previously received at least one platinum-containing chemotherapy regimen.

39. The method of claim 38, wherein the subject had disease progression during or following treatment with at least one platinum-containing chemotherapy regimen.

40. The method of claim 39, wherein the at least one platinum-containing chemotherapy regimen comprised (i) gemcitabine and cisplatin or carboplatin or (ii) methotrexate, vinblastine, doxorubicin, and cisplatin.

41. The method of claim 38, wherein the subject received prior adjuvant or neoadjuvant chemotherapy and progressed within 12 months of treatment with a platinum-containing adjuvant or neoadjuvant regimen.

42. The method of claim 38, wherein the subject received one cycle of a platinum-containing chemotherapy regimen but discontinued because of a Grade 4 hematologic toxicity or a Grade 3-4 non-hematologic toxicity.

43. The method of any one of claims 33-42, wherein the subject received no more than two prior lines of treatment for the locally advanced or metastatic UC.

44. The method of any one of claims 33-43, wherein the subject has not received prior treatment with a T-cell co-stimulating therapy or a checkpoint inhibitor.

45. The method of any one of claims 1-44, wherein the anti-latent TGF-beta 1 antibody is administered to the subject in combination with one or more additional therapeutic agents.

46. The method of claim 45, wherein the one or more additional therapeutic agents comprises a checkpoint inhibitor.

47. The method of any one of claims 15, 29, 32, 44, and 46, wherein the checkpoint inhibitor comprises a PD-1 axis binding antagonist or a CTLA4 antagonist.

48. The method of claim 47, wherein the checkpoint inhibitor comprises a PD-1 axis binding antagonist.

49. The method of any one of claims 20-22, 47, and 48, wherein the PD-1 axis binding antagonist comprises a PD-L1 binding antagonist, a PD-1 binding antagonist, or a PD-L2 binding antagonist.

50. The method of claim 49, wherein the PD-1 axis binding antagonist comprises a PD-L1 binding antagonist.

51. The method of claim 50, wherein the PD-L1 binding antagonist comprises an anti-PD-L1 antibody.

52. The method of claim 51, wherein the anti-PD-L1 antibody comprises atezolizumab, durvalumab, avelumab, or MDX-1105.

53. The method of claim 52, wherein the anti-PD-L1 antibody comprises atezolizumab.

54. The method of claim 53, wherein the atezolizumab is administered to the subject in a dosing regimen comprising one or more dosing cycles.

55. The method of claim 54, wherein the one or more dosing cycles comprise 21-day dosing cycles.

56. The method of claim 55, wherein the atezolizumab is administered to the subject on Day 1 of each 21-day dosing cycle.

57. The method of any one of claims 53-56, wherein the atezolizumab is administered to the subject at a dose of 1200 mg.

58. The method of claim 54, wherein the one or more dosing cycles comprise 14-day dosing cycles or 28-day dosing cycles.

59. The method of claim 58, wherein the one or more dosing cycles comprise 14-day dosing cycles, and the atezolizumab is administered to the subject at a dose of 840 mg.

60. The method of claim 59, wherein the atezolizumab is administered to the subject on Day 1 of each 14-day dosing cycle.

61. The method of claim 58, wherein the one or more dosing cycles comprise 28-day dosing cycles, and the atezolizumab is administered to the subject at a dose of 1680 mg.

62. The method of claim 61, wherein the atezolizumab is administered to the subject on Day 1 of each 28-day dosing cycle.

63. The method of any one of claims 53-62, wherein the atezolizumab is administered to the subject intravenously.

64. The method of claim 63, wherein the atezolizumab is administered to the subject intravenously by infusion.

65. The method of claim 49, wherein the PD-1 axis binding antagonist comprises a PD-1 binding antagonist.

66. The method of claim 65, wherein the PD-1 binding antagonist comprises an anti-PD-1 antibody.

67. The method of claim 66, wherein the anti-PD-1 antibody comprises nivolumab, pembrolizumab, MEDI-0680, spartalizumab, cemiplimab, prolgolimab, camrelizumab, sintilimab, tislelizumab, toripalimab, dostarlimab, retifanlimab, sasanlimab, penpulimab, zimberelimab, balstilimab, genolimzumab, cetrelimab, or budigalimab.

68. The method of claim 67, wherein the anti-PD-1 antibody comprises nivolumab.

69. The method of claim 68, wherein the nivolumab is administered to the subject in a dosing regimen comprising one or more dosing cycles.

70. The method of claim 69, wherein the one or more dosing cycles comprise 21-day dosing cycles.

71. The method of claim 70, wherein the nivolumab is administered to the subject on Day 1 of each 21-day dosing cycle.

72. The method of any one of claims 68-71, wherein the nivolumab is administered to the subject at a dose of 360 mg.

73. The method of any one of claims 68-72, wherein the nivolumab is administered to the subject intravenously.

74. The method of claim 73, wherein the nivolumab is administered to the subject intravenously by infusion.

75. The method of any one of claims 1-74, wherein the one or more additional therapeutic agents is selected from a chemotherapeutic agent, an immunotherapy agent, a radiation therapy agent, an anti-angiogenic agent, and any combination thereof.

76. The method of claim 75, wherein the one or more additional therapeutic agents comprise one or more chemotherapeutic agents.

77. The method of claim 76, wherein the one or more chemotherapeutic agents comprise a platinum-based chemotherapeutic agent, an antimetabolite, a cytotoxic agent, a growth inhibitory agent, a taxane, a folate analog, or any combination thereof.

78. The method of claim 77, wherein the platinum-based chemotherapeutic agent comprises oxaliplatin, cisplatin, or carboplatin.

79. The method of claim 78, wherein the platinum-based chemotherapeutic agent comprises oxaliplatin.

80. The method of claim 79, wherein the oxaliplatin is administered to the subject in a dosing regimen comprising one or more dosing cycles.

81. The method of claim 80, wherein the one or more dosing cycles comprise 21-day dosing cycles.

82. The method of claim 81, wherein the oxaliplatin is administered to the subject on Day 1 of each 21-day dosing cycle.

83. The method of any one of claims 79-82, wherein the oxaliplatin is administered to the subject at a dose of 130 mg/m².

84. The method of any one of claims 79-83, wherein the oxaliplatin is administered to the subject intravenously.

85. The method of claim 84, wherein the antimetabolite comprises capecitabine, gemcitabine, 5-fluorouracil, or tegafur.

86. The method of claim 85, wherein the antimetabolite comprises capecitabine.

87. The method of claim 86, wherein the capecitabine is administered to the subject in a dosing regimen comprising one or more dosing cycles.

88. The method of claim 87, wherein the one or more dosing cycles comprise 21-day dosing cycles.

89. The method of claim 88, wherein the capecitabine is administered to the subject on Days 1-14 of each 21-day dosing cycle.

90. The method of any one of claims 86-89, wherein the capecitabine is administered to the subject at a dose of 1000 mg/m² twice daily.

91. The method of any one of claims 86-90, wherein the capecitabine is administered to the subject orally.

92. The method of claim 85, wherein the antimetabolite comprises gemcitabine.

93. The method of claim 92, wherein the gemcitabine is administered to the subject in a dosing regimen comprising one or more dosing cycles.

94. The method of claim 93, wherein the one or more dosing cycles comprise 28-day dosing cycles.

95. The method of claim 94, wherein the gemcitabine is administered to the subject on Days 1, 8, and 15 of each 28-day dosing cycle.

96. The method of any one of claims 92-95, wherein the gemcitabine is administered to the subject at a dose of 1000 mg/m² twice daily.

97. The method of any one of claims 92-96, wherein the gemcitabine is administered to the subject intravenously.

98. The method of claim 85, wherein the antimetabolite comprises tegafur.

99. The method of claim 98, wherein the antimetabolite comprises S-1 (tegafur-gimeracil-oteracil potassium).

100. The method of claim 99, wherein the S-1 is administered to the subject in a dosing regimen comprising one or more dosing cycles.

101. The method of claim 100, wherein the one or more dosing cycles comprise 21-day dosing cycles.

102. The method of claim 101, wherein the S-1 is administered to the subject on Days 1-14 of each 21-day dosing cycle.

103. The method of any one of claims 99-102, wherein the S-1 is administered to the subject at a dose of 40 mg/m² twice daily.

104. The method of any one of claims 99-103, wherein the S-1 is administered to the subject orally.

105. The method of claim 77, wherein the taxane comprises nab-paclitaxel or paclitaxel.

106. The method of claim 105, wherein the taxane comprises nab-paclitaxel.

107. The method of claim 106, wherein the nab-paclitaxel is administered to the subject in a dosing regimen comprising one or more dosing cycles.

108. The method of claim 107, wherein the one or more dosing cycles comprise 28-day dosing cycles.

109. The method of claim 108, wherein the nab-paclitaxel is administered to the subject on Days 1, 8, and 15 of each 28-day dosing cycle.

110. The method of any one of claims 105-109, wherein the nab-paclitaxel is administered to the subject at a dose of 125 mg/m².

111. The method of any one of claims 105-110, wherein the nab-paclitaxel is administered to the subject intravenously.

112. The method of claim 77, wherein the folate analog comprises leucovorin.

113. A method of treating a subject having a locally advanced, recurrent, or metastatic NSCLC, the method comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and
(ii) atezolizumab at a dosage of 1200 mg intravenously on Day 1 of each 21-day dosing cycle.

114. The method of claim 113, wherein the NSCLC is histologically or cytologically confirmed metastatic nonsquamous NSCLC or metastatic squamous NSCLC.

115. The method of claim 113 or 114, wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

116. The method of claim 115, wherein the subject has previously received a combination therapy comprising a platinum-containing chemotherapy regimen and a PD-1 axis binding antagonist.

117. The method of claim 115, wherein the subject has previously received a platinum-containing chemotherapy regimen and a PD-1 axis binding antagonist as individual regimens.

118. The method of any one of claims 113-117, wherein the subject had disease progression or recurrence within 6 months of definitive therapy for locally advanced NSCLC.

119. The method of any one of claims 113-118, wherein a tumor sample from the subject has been determined to have a detectable level of PD-L1 expression.

120. A method of treating a subject having a locally advanced, recurrent, or metastatic gastric cancer, the method comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and
(ii) nivolumab at a dosage of 360 mg intravenously on Day 1 of each 21-day dosing cycle;
(iii)
(a) capecitabine at a dosage of 1000 mg/m² orally twice daily on Days 1-14 of each 21-day dosing cycle, or
(b) S-1 at a dosage of 40 mg/m² orally twice daily on Days 1-14 of each 21-day dosing cycle; and
(iv) oxaliplatin at a dosage of 130 mg/m² intravenously on Day 1 of each 21-day dosing cycle.

121. The method of claim 120, wherein the subject has unresectable locally advanced or metastatic gastric cancer that is histologically confirmed to be adenocarcinoma.

122. The method of claim 120 or 121, wherein the gastric cancer comprises esophagogastric junction cancer.

123. The method of any one of claims 120-122, wherein the gastric cancer is HER2-negative gastric cancer.

124. The method of any one of claims 120-123, wherein the subject is previously untreated for gastric cancer and/or the subject has not been previously treated with a checkpoint inhibitor.

125. A method of treating a subject having a locally advanced, recurrent, or metastatic PDAC, the method comprising administering to the subject an anti-cancer therapy in a dosing regimen comprising one or more 28-day dosing cycles, wherein the anti-cancer therapy comprises:
(i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively;
(ii) atezolizumab at a dosage of 840 mg intravenously on Days 1 and 15 of each 28-day dosing cycle;
(iii) nab-paclitaxel at a dose of 125 mg/m² on Days 1, 8, and 15 of each 28-day dosing cycle; and
(iv) gemcitabine at a dosage of 1000 mg/m² intravenously on Days 1, 8, and 15 of each 28-day dosing cycle.

126. The method of claim 125, wherein the subject has histologically or cytologically confirmed metastatic PDAC.

127. The method of claim 125 or 126, wherein the subject is previously untreated for the PDAC and/or the subject has not been previously treated with a checkpoint inhibitor.

128. A method of treating a subject having a locally advanced, recurrent, or metastatic UC, the method comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and
(ii) atezolizumab at a dosage of 1200 mg intravenously on Day 1 of each 21-day dosing cycle.

129. The method of claim 128, wherein the subject has histologically documented, locally advanced (T4b, any N; or any T, N2-N3) UC, or metastatic UC (M1, Stage 4).

130. The method of claim 128 or 129, wherein the subject is previously untreated for UC.

131. The method of any one of claims 128-130, wherein the subject is ineligible for cisplatin-containing chemotherapy.

132. The method of claim 131, wherein the subject is ineligible for cisplatin-containing chemotherapy as defined by any one of the following criteria:
(i) impaired renal function in terms of glomerular filtration rate (GFR) of ≥30 mL/min but <60 mL/min as assessed by direct measurement or by calculation from serum or plasma creatinine;
(ii) hearing loss of 25 dB at two contiguous frequencies as measured by audiometry;
(iii) Grade 2 peripheral neuropathy; or
(iv) ECOG Performance Status of 2.

133. The method of any one of claims 128-130, wherein the subject has previously received at least one platinum-containing chemotherapy regimen.

134. The method of claim 133, wherein the subject had disease progression during or following treatment with at least one platinum-containing chemotherapy regimen.

135. The method of claim 134, wherein the at least one platinum-containing chemotherapy regimen comprised (i) gemcitabine and cisplatin or carboplatin or (ii) methotrexate, vinblastine, doxorubicin, and cisplatin.

136. The method of claim 133, wherein the subject received prior adjuvant or neoadjuvant chemotherapy and progressed within 12 months of treatment with a platinum-containing adjuvant or neoadjuvant regimen.

137. The method of claim 133, wherein the subject received one cycle of a platinum-containing chemotherapy regimen but discontinued because of a Grade 4 hematologic toxicity or a Grade 3-4 non-hematologic toxicity.

138. The method of any one of claims 128-137, wherein the subject received no more than two prior regimens of treatment for the locally advanced or metastatic UC.

139. The method of any one of claims 128-138, wherein the subject has not received prior treatment with a T-cell co-stimulating therapy or a checkpoint inhibitor.

140. A method of treating a subject having a metastatic non-squamous NSCLC or a metastatic squamous NSCLC, the method comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) an HVR-H1 comprising the amino acid sequence of SEAMN (SEQ ID NO: 1);
(b) an HVR-H2 comprising the amino acid sequence of YIYTSGTTYRANWARG (SEQ ID NO: 2);
(c) an HVR-H3 comprising the amino acid sequence of GTGIYDYYYWVMDL (SEQ ID NO:3);
(d) an HVR-L1 comprising the amino acid sequence of QASQSISTYLA (SEQ ID NO: 4);
(e) an HVR-L2 comprising the amino acid sequence of AASTLES (SEQ ID NO: 5); and
(f) an HVR-L3 comprising the amino acid sequence of QSYSDGDSVG (SEQ ID NO: 6); and
(ii) atezolizumab at a dosage of 1200 mg intravenously on Day 1 of each 21-day dosing cycle,
wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

141. A method of treating a subject having a locally advanced, unresectable, or metastatic HER2-negative gastric cancer, the method comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) an HVR-H1 comprising the amino acid sequence of SEAMN (SEQ ID NO: 1);
(b) an HVR-H2 comprising the amino acid sequence of YIYTSGTTYRANWARG (SEQ ID NO: 2);
(c) an HVR-H3 comprising the amino acid sequence of GTGIYDYYYWVMDL (SEQ ID NO:3);
(d) an HVR-L1 comprising the amino acid sequence of QASQSISTYLA (SEQ ID NO: 4);
(e) an HVR-L2 comprising the amino acid sequence of AASTLES (SEQ ID NO: 5); and
(f) an HVR-L3 comprising the amino acid sequence of QSYSDGDSVG (SEQ ID NO: 6);
(ii) nivolumab at a dosage of 360 mg intravenously on Day 1 of each 21-day dosing cycle;
(iii)
(a) capecitabine at a dosage of 1000 mg/m² orally twice daily on Days 1-14 of each 21-day dosing cycle, or
(b) S-1 at a dosage of 40 mg/m² orally twice daily on Days 1-14 of each 21-day dosing cycle; and
(iv) oxaliplatin at a dosage of 130 mg/m² intravenously on Day 1 of each 21-day dosing cycle,
wherein the subject is previously untreated for the locally advanced, unresectable, or metastatic HER2-negative gastric cancer.

142. A method of treating a subject having a metastatic PDAC, the method comprising administering to the subject an anti-cancer therapy in a dosing regimen comprising one or more 28-day dosing cycles, wherein the anti-cancer therapy comprises:
(i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs:
(a) an HVR-H1 comprising the amino acid sequence of SEAMN (SEQ ID NO: 1);
(b) an HVR-H2 comprising the amino acid sequence of YIYTSGTTYRANWARG (SEQ ID NO: 2);
(c) an HVR-H3 comprising the amino acid sequence of GTGIYDYYYWVMDL (SEQ ID NO:3);
(d) an HVR-L1 comprising the amino acid sequence of QASQSISTYLA (SEQ ID NO: 4);
(e) an HVR-L2 comprising the amino acid sequence of AASTLES (SEQ ID NO: 5); and
(f) an HVR-L3 comprising the amino acid sequence of QSYSDGDSVG (SEQ ID NO: 6);
(ii) atezolizumab at a dosage of 840 mg intravenously on Days 1 and 15 of each 28-day dosing cycle;
(iii) nab-paclitaxel at a dose of 125 mg/m² on Days 1, 8, and 15 of each 28-day dosing cycle; and
(iv) gemcitabine at a dosage of 1000 mg/m² intravenously on Days 1, 8, and 15 of each 28-day dosing cycle,
wherein the subject is previously untreated for the metastatic PDAC.

143. A method of treating a subject having a locally advanced or metastatic UC, the method comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) an HVR-H1 comprising the amino acid sequence of SEAMN (SEQ ID NO: 1);
(b) an HVR-H2 comprising the amino acid sequence of YIYTSGTTYRANWARG (SEQ ID NO: 2);
(c) an HVR-H3 comprising the amino acid sequence of GTGIYDYYYWVMDL (SEQ ID NO:3);
(d) an HVR-L1 comprising the amino acid sequence of QASQSISTYLA (SEQ ID NO: 4);
(e) an HVR-L2 comprising the amino acid sequence of AASTLES (SEQ ID NO: 5); and
(f) an HVR-L3 comprising the amino acid sequence of QSYSDGDSVG (SEQ ID NO: 6); and
(ii) atezolizumab at a dosage of 1200 mg intravenously on Day 1 of each 21-day dosing cycle, wherein:
the subject is previously untreated for the locally advanced or metastatic UC and is ineligible for cisplatin-containing chemotherapy; or
the subject has previously received at least one platinum-containing chemotherapy regimen.

144. The method of any one of claims 1-143, wherein the subject is administered the anti-latent TGF-beta 1 antibody until loss of clinical benefit or unacceptable toxicity.

145. The method of any one of claims 1-144, wherein the method comprises between 1 and 20 dosing cycles.

146. The method of any one of claims 1-145, wherein the anti-latent TGF-beta 1 antibody comprises HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively.

147. The method of any one of claims 1-146, wherein the anti-latent TGF-beta 1 antibody comprises:
(a) (i) a heavy chain variable domain (VH) sequence having at least 95% sequence identity to the amino acid sequence of SEQ ID NO: 7, (ii) a light chain variable domain (VL) sequence having at least 95% sequence identity to the amino acid sequence of SEQ ID NO: 8, or (iii) a VH sequence as defined in (i) and a VL sequence as defined in (ii);
(b) (i) a VH sequence having at least 95% sequence identity to the amino acid sequence of SEQ ID NO: 31, (ii) a VL sequence having at least 95% sequence identity to the amino acid sequence of SEQ ID NO: 32, or (iii) a VH sequence as defined in (i) and a VL sequence as defined in (ii);
(c) (i) a VH sequence having at least 95% sequence identity to the amino acid sequence of SEQ ID NO: 33, (ii) a VL sequence having at least 95% sequence identity to the amino acid sequence of SEQ ID NO: 34, or (iii) a VH sequence as defined in (i) and a VL sequence as defined in (ii); or
(d) (i) a VH sequence having at least 95% sequence identity to the amino acid sequence of SEQ ID NO: 35, (ii) a VL sequence having at least 95% sequence identity to the amino acid sequence of SEQ ID NO: 36, or (iii) a VH sequence as defined in (i) and a VL sequence as defined in (ii).

148. The method of any one of claims 1-147, wherein the anti-latent TGF-beta 1 antibody comprises:
(a) a VH sequence of SEQ ID NO: 7 and a VL sequence of SEQ ID NO: 8;
(b) a VH sequence of SEQ ID NO: 31 and a VL sequence of SEQ ID NO: 32;
(c) a VH sequence of SEQ ID NO: 33 and a VL sequence of SEQ ID NO: 34; or
(d) a VH sequence of SEQ ID NO: 35 and a VL sequence of SEQ ID NO: 36.

149. The method of any one of claims 1-148, wherein the anti-latent TGF-beta 1 antibody comprises a VH sequence of SEQ ID NO: 7 and a VL sequence of SEQ ID NO: 8.

150. The method of any one of claims 1-149, wherein the anti-latent TGF-beta 1 antibody is a chimeric antibody.

151. The method of any one of claims 1-150, wherein the anti-latent TGF-beta 1 antibody is a humanized antibody.

152. The method of any one of claims 1-151, wherein the anti-latent TGF-beta 1 antibody is a full-length antibody.

153. The method of any one of claims 1-152, wherein the anti-latent TGF-beta 1 antibody comprises:
(a) a heavy chain comprising the amino acid sequence of SEQ ID NO: 37 and a light chain comprising the amino acid sequence of SEQ ID NO: 38;
(b) a heavy chain comprising the amino acid sequence of SEQ ID NO: 39 and a light chain comprising the amino acid sequence of SEQ ID NO: 40;
(c) a heavy chain comprising the amino acid sequence of SEQ ID NO: 41 and a light chain comprising the amino acid sequence of SEQ ID NO: 42;
(d) a heavy chain comprising the amino acid sequence of SEQ ID NO: 43 and a light chain comprising the amino acid sequence of SEQ ID NO: 44;
(e) a heavy chain comprising the amino acid sequence of SEQ ID NO: 45 and a light chain comprising the amino acid sequence of SEQ ID NO: 46;
(f) a heavy chain comprising the amino acid sequence of SEQ ID NO: 47 and a light chain comprising the amino acid sequence of SEQ ID NO: 48;
(g) a heavy chain comprising the amino acid sequence of SEQ ID NO: 49 and a light chain comprising the amino acid sequence of SEQ ID NO: 50; or
(h) a heavy chain comprising the amino acid sequence of SEQ ID NO: 51 and a light chain comprising the amino acid sequence of SEQ ID NO: 52.

154. The method of any one of claims 1-152, wherein the anti-latent TGF-beta 1 antibody comprises a modified IgG1 Fc region having reduced effector function compared with a wild-type IgG1 Fc region.

155. The method of claim 154, wherein the modified IgG1 Fc region comprises a constant heavy (CH) region comprising one or more of the following substitutions: K214R, L235R, G236R, M428L, N434A, Q438R, S440E (EU numbering).

156. The method of claim 155, wherein the CH region comprises the amino acid sequence of SEQ ID NO: 9.

157. The method of any one of claims 152-156, wherein the modified IgG1 Fc region comprises a constant light (CL) domain comprising the amino acid sequence of SEQ ID NO: 10.

158. The method of any one of claims 1-157, wherein the anti-latent TGF-beta 1 antibody comprises a heavy chain comprising the amino acid sequence of SEQ ID NO: 11 and a light chain sequence comprising the amino acid sequence of SEQ ID NO: 12.

159. The method of any one of claims 1-151, wherein the anti-latent TGF-beta 1 antibody is an antibody fragment that binds to latent TGF-beta 1.

160. The method of any one of claims 1-159, wherein the subject is a human.

161. An anti-latent TGF-beta 1 for use in treatment of a subject having a locally advanced, recurrent, or metastatic solid tumor, the treatment comprising administering to the subject an anti-cancer therapy comprising an anti-latent TGF-beta 1 antibody at a dosage of 1800 mg, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

162. An anti-latent TGF-beta 1 for use in treatment of a subject having a locally advanced, recurrent, or metastatic solid tumor, the treatment comprising administering to the subject an anti-cancer therapy comprising an anti-latent TGF-beta 1 antibody at a dosage of 1800 mg every three weeks (Q3W), wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

163. An anti-latent TGF-beta 1 for use in treatment of a subject having a locally advanced, recurrent, or metastatic solid tumor, the treatment comprising administering to the subject an anti-cancer therapy comprising an anti-latent TGF-beta 1 antibody at a dosage of 1200 mg, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

164. An anti-latent TGF-beta 1 for use in treatment of a subject having a locally advanced, recurrent, or metastatic solid tumor, the treatment comprising administering to the subject an anti-cancer therapy comprising an anti-latent TGF-beta 1 antibody at a dosage of 1200 mg every two weeks (Q2W), wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively.

165. An anti-latent TGF-beta 1 for use in treatment of a subject having a locally advanced, recurrent, or metastatic NSCLC, the treatment comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and
(ii) atezolizumab at a dosage of 1200 mg intravenously on Day 1 of each 21-day dosing cycle.

166. An anti-latent TGF-beta 1 for use in treatment of a subject having a locally advanced, recurrent, or metastatic gastric cancer, the treatment comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively;
(ii) nivolumab at a dosage of 360 mg intravenously on Day 1 of each 21-day dosing cycle;
(iii)
(a) capecitabine at a dosage of 1000 mg/m² orally twice daily on Days 1-14 of each 21-day dosing cycle, or
(b) S-1 at a dosage of 40 mg/m² orally twice daily on Days 1-14 of each 21-day dosing cycle; and
(iv) oxaliplatin at a dosage of 130 mg/m² intravenously on Day 1 of each 21-day dosing cycle.

167. An anti-latent TGF-beta 1 for use in treatment of a subject having a locally advanced, recurrent, or metastatic PDAC, the treatment comprising administering to the subject an anti-cancer therapy in a dosing regimen comprising one or more 28-day dosing cycles, wherein the anti-cancer therapy comprises:
(i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively;
(ii) atezolizumab at a dosage of 840 mg intravenously on Days 1 and 15 of each 28-day dosing cycle;
(iii) nab-paclitaxel at a dose of 125 mg/m² on Days 1, 8, and 15 of each 28-day dosing cycle; and
(iv) gemcitabine at a dosage of 1000 mg/m² intravenously on Days 1, 8, and 15 of each 28-day dosing cycle.

168. An anti-latent TGF-beta 1 for use in treatment of a subject having a locally advanced, recurrent, or metastatic UC, the treatment comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively; and
(ii) atezolizumab at a dosage of 1200 mg intravenously on Day 1 of each 21-day dosing cycle.

169. An anti-latent TGF-beta 1 for use in treatment of a subject having a metastatic non-squamous NSCLC or a metastatic squamous NSCLC, the treatment comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) an HVR-H1 comprising the amino acid sequence of SEAMN (SEQ ID NO: 1);
(b) an HVR-H2 comprising the amino acid sequence of YIYTSGTTYRANWARG (SEQ ID NO: 2);
(c) an HVR-H3 comprising the amino acid sequence of GTGIYDYYYWVMDL (SEQ ID NO:3);
(d) an HVR-L1 comprising the amino acid sequence of QASQSISTYLA (SEQ ID NO: 4);
(e) an HVR-L2 comprising the amino acid sequence of AASTLES (SEQ ID NO: 5); and
(f) an HVR-L3 comprising the amino acid sequence of QSYSDGDSVG (SEQ ID NO: 6); and
(ii) atezolizumab at a dosage of 1200 mg intravenously on Day 1 of each 21-day dosing cycle,
wherein the subject has had disease progression during or following treatment for metastatic or locally advanced, inoperable NSCLC that comprised a platinum-containing chemotherapy regimen and a PD-1 axis binding antagonist, given in combination as one line of therapy or as two separate lines of therapy, in either order, for a maximum of two prior lines of systemic therapy.

170. An anti-latent TGF-beta 1 for use in treatment of a subject having a locally advanced, unresectable, or metastatic HER2-negative gastric cancer, the treatment comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) an HVR-H1 comprising the amino acid sequence of SEAMN (SEQ ID NO: 1);
(b) an HVR-H2 comprising the amino acid sequence of YIYTSGTTYRANWARG (SEQ ID NO: 2);
(c) an HVR-H3 comprising the amino acid sequence of GTGIYDYYYWVMDL (SEQ ID NO:3);
(d) an HVR-L1 comprising the amino acid sequence of QASQSISTYLA (SEQ ID NO: 4);
(e) an HVR-L2 comprising the amino acid sequence of AASTLES (SEQ ID NO: 5); and
(f) an HVR-L3 comprising the amino acid sequence of QSYSDGDSVG (SEQ ID NO: 6);
(ii) nivolumab at a dosage of 360 mg intravenously on Day 1 of each 21-day dosing cycle;
(iii)
(a) capecitabine at a dosage of 1000 mg/m² orally twice daily on Days 1-14 of each 21-day dosing cycle, or
(b) S-1 at a dosage of 40 mg/m² orally twice daily on Days 1-14 of each 21-day dosing cycle; and
(iv) oxaliplatin at a dosage of 130 mg/m² intravenously on Day 1 of each 21-day dosing cycle,
wherein the subject is previously untreated for the locally advanced, unresectable, or metastatic HER2-negative gastric cancer.

171. An anti-latent TGF-beta 1 for use in treatment of a subject having a metastatic PDAC, the treatment comprising administering to the subject an anti-cancer therapy in a dosing regimen comprising one or more 28-day dosing cycles, wherein the anti-cancer therapy comprises:
(i) an anti-latent TGF-beta 1 antibody at a dosage of 1200 mg intravenously on Days 1 and 15 of each 28-day dosing cycle, wherein the anti-latent TGF-beta 1 antibody comprises the following six HVRs:
(a) an HVR-H1 comprising the amino acid sequence of SEAMN (SEQ ID NO: 1);
(b) an HVR-H2 comprising the amino acid sequence of YIYTSGTTYRANWARG (SEQ ID NO: 2);
(c) an HVR-H3 comprising the amino acid sequence of GTGIYDYYYWVMDL (SEQ ID NO:3);
(d) an HVR-L1 comprising the amino acid sequence of QASQSISTYLA (SEQ ID NO: 4);
(e) an HVR-L2 comprising the amino acid sequence of AASTLES (SEQ ID NO: 5); and
(f) an HVR-L3 comprising the amino acid sequence of QSYSDGDSVG (SEQ ID NO: 6);
(ii) atezolizumab at a dosage of 840 mg intravenously on Days 1 and 15 of each 28-day dosing cycle;
(iii) nab-paclitaxel at a dose of 125 mg/m² on Days 1, 8, and 15 of each 28-day dosing cycle; and
(iv) gemcitabine at a dosage of 1000 mg/m² intravenously on Days 1, 8, and 15 of each 28-day dosing cycle,
wherein the subject is previously untreated for the metastatic PDAC.

172. An anti-latent TGF-beta 1 for use in treatment of a subject having a locally advanced or metastatic UC, the treatment comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1800 mg intravenously on Day 1 of each 21-day dosing cycle, wherein the anti-latent TGF-beta 1 antibody comprises the following six HVRs:
(a) an HVR-H1 comprising the amino acid sequence of SEAMN (SEQ ID NO: 1);
(b) an HVR-H2 comprising the amino acid sequence of YIYTSGTTYRANWARG (SEQ ID NO: 2);
(c) an HVR-H3 comprising the amino acid sequence of GTGIYDYYYWVMDL (SEQ ID NO:3);
(d) an HVR-L1 comprising the amino acid sequence of QASQSISTYLA (SEQ ID NO: 4);
(e) an HVR-L2 comprising the amino acid sequence of AASTLES (SEQ ID NO: 5); and
(f) an HVR-L3 comprising the amino acid sequence of QSYSDGDSVG (SEQ ID NO: 6); and
(ii) atezolizumab at a dosage of 1200 mg intravenously on Day 1 of each 21-day dosing cycle, wherein:
the subject is previously untreated for the locally advanced or metastatic UC and is ineligible for cisplatin-containing chemotherapy; or
the subject has previously received at least one platinum-containing chemotherapy regimen.

173. A method of treating cancer in a subject having a locally advanced or recurrent tumor, the method comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1200 mg to 1800 mg intravenously on Day 1 of each 21-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively, and
(ii) atezolizumab at a dosage of 1200 mg intravenously on Day 1 of each 21-day dosing cycle.

174. The method of claim 173, wherein the subject has previously received treatment with atezolizumab at a dosage of 1200 mg intravenously.

175. The method of claim 173 or 174, wherein the anti-cancer therapy comprises the anti-latent TGF-beta 1 antibody at a dosage of 1200 mg.

176. The method of any one of claims 173-175, wherein the anti-cancer therapy comprises the anti-latent TGF-beta 1 antibody at a dosage of 1500 mg.

177. The method of any one of claims 173-176, wherein the anti-cancer therapy comprises the anti-latent TGF-beta 1 antibody at a dosage of 1800 mg.

178. The method of any one of claims 173-177, wherein the anti-latent TGF-beta 1 antibody is administered to the subject intravenously.

179. The method of claim 178, wherein the anti-latent TGF-beta 1 antibody is administered to the subject intravenously by infusion.

180. The method of any one of claims 173-179, wherein the has been determined to have a detectable level of PD-L1 expression.

181. The method of any one of claims 173-180, wherein the subject's age is 18 years or older.

182. The method of any one of claims 173-181, wherein the anti-cancer therapy is a first-line therapy.

183. The method of any one of claims 173-181, wherein the anti-cancer therapy is a second-line therapy or a third-line therapy.

184. The method of any one of claims 173-183, wherein the subject has not been previously treated with a checkpoint inhibitor.

185. The method of any one of claims 173-184, wherein the cancer is NSCLC, gastric cancer, PDAC, UC, GIST, skin cancer, colorectal cancer, OV cancer, renal cancer, or gallbladder cancer.

186. The method of claim 185, wherein the cancer is GIST, skin cancer, colorectal cancer, OV cancer, renal cancer, or gallbladder cancer.

187. An anti-latent TGF-beta 1 for use in a treatment of cancer in a subject having a locally advanced or recurrent tumor, the treatment comprising administering to the subject an anti-cancer therapy in a dosing regimen comprising one or more 21-day dosing cycles, wherein the anti-cancer therapy comprises:
(i) an anti-latent TGF-beta 1 antibody at a dosage of 1200 mg to 1800 mg intravenously on Day 1 of each 21-day dosing cycle, wherein the anti-latent TGF-beta 1 antibody comprises the following six HVRs:
(a) HVR-H1, HVR-H2, and HVR-H3 comprising the amino acid sequences of SEQ ID NO: 1, 2, and 3, respectively, and HVR-L1, HVR-L2, and HVR-L3 comprising the amino acid sequences of SEQ ID NO: 4, 5, and 6, respectively;
(b) HVR-H1, HVR-H2, and HVR-H3 comprising the amino acid sequences of SEQ ID NO: 13, 14, and 15, respectively, and HVR-L1, HVR-L2, and HVR-L3 comprising the amino acid sequences of SEQ ID NO: 16, 17, and 18, respectively;
(c) HVR-H1, HVR-H2, and HVR-H3 comprising the amino acid sequences of SEQ ID NO: 19, 20, and 21, respectively, and HVR-L1, HVR-L2, and HVR-L3 comprising the amino acid sequences of SEQ ID NO: 22, 23, and 24, respectively; or
(d) HVR-H1, HVR-H2, and HVR-H3 comprising the amino acid sequences of SEQ ID NO: 25, 26, and 27, respectively, and HVR-L1, HVR-L2, and HVR-L3 comprising the amino acid sequences of SEQ ID NO: 28, 29, and 30, respectively, and
(ii) atezolizumab at a dosage of 1200 mg intravenously on Day 1 of each 21-day dosing cycle.
